(19) United States
(12) Patent Application Publication
(10) Pub. No.: US 2023/0219925 A1
(43) Pub. Date: Jul. 13, 2023

(54) BENZO FIVE-MEMBERED NITROGEN HETEROCYCLIC COMPOUND AND APPLICATION THEREOF

(71) Applicant: GUANGZHOU INSTITUTES OF BIOMEDICINE AND HEALTH CHINESE ACADEMY OF SCIENCES, Guangdong (CN)

(72) Inventors: Yong XU, Guangdong (CN); Xishan WU, Guangdong (CN); Yan ZHANG, Guangdong (CN); Rui WANG, Guangdong (CN); Hui SHEN, Guangdong (CN)

(21) Appl. No.: 18/009,899

(22) PCT Filed: Sep. 8, 2020
(86) PCT No.: PCT/CN2020/113949

§ 371 Date: Dec. 12, 2022

(30) Foreign Application Priority Data
Jul. 10, 2020 (CN) 202010663949.5

(51) Int. Cl. C07D 401/06; A61P 35/00; C07D 277/66; C07D 417/12; C07D 235/18; C07D 401/04

(52) U.S. Cl. CPC C07D 401/06; A61P 35/00; C07D 277/66; C07D 417/12; C07D 235/18; C07D 401/04

(57) ABSTRACT

The present application relates to a benzo five-membered nitrogen heterocyclic compound and an application thereof. The benzo five-membered nitrogen heterocyclic compound has the structure represented by formula I. The compound can be used as a compound of RORγ receptor inhibitor. The compound can effectively inhibit RORγ proteins and have good selectivity to other nuclear receptor family proteins. The benzo five-membered nitrogen heterocyclic compound or a pharmaceutical composition thereof provided by the present application can be used for preparing a drug for treating, preventing or ameliorating diseases such as inflammations, autoimmune diseases, cell proliferative disorder diseases, sepsis, cancer, neurodegeneration diseases or viral infections, has a good inhibitory effect on the treatment of tumors, especially the treatment of prostate cancer, and also has an amelioration effect on the treatment of other diseases.

### TECHNICAL FIELD

The present application relates to the technical field of chemical medicines and, in particular, to a benzo five-membered nitrogen heterocyclic compound and a use thereof.

### BACKGROUND

Retinoic acid receptor-related orphan receptor (ROR) is an important type of orphan receptors in a nuclear receptor family. The receptor family includes three members, namely RORα (NR1F1), RORβ (NR1F2) and RORγ (NR1F3), which are distributed in different tissues and organs of a body, respectively. RORα is widely expressed in a skeletal muscle, a liver, a lung, a skin, an adipocyte tissue, a kidney, a thymus and a brain. An expression site of RORβ is very limited, that is, only in a central nervous system is RORβ expressed. RORγ has two subtypes: RORγ1 and RORγ2. The latter one is also known as RORγt. RORγ1 is highly expressed in the skeletal muscle, the liver, the kidney and the adipose tissue. Only in immune tissues including the thymus is RORγt highly expressed.

TH17 cells are a subset of TH cells capable of secreting interleukin 17 (IL-17). As a proinflammatory factor, IL-17 plays an important role in autoimmune diseases and the development of inflammations. Therefore, an immune system response can be regulated through a regulation of TH17 cells differentiation and IL-17 secretion. In 2006, Professor Littman of New York University first discovered that RORγ can directly promote the differentiation and development of TH17 cells. RORγ directly regulates generation and secretion levels of IL-17 cytokines and is a key factor in the development of TH17 cells. Therefore, inhibition of RORγ transcription is expected to be a new strategy to be selected for the treatment of autoimmune diseases.

Previous studies have found that the nuclear receptor RORγ is highly expressed in metastatic castration-resistant prostate cancer (CRPC), acts on an upstream of an androgen receptor (AR) gene and regulates expressions of the AR and related genes regulated by the AR. XY011, XY018 and XY101, which are obtained through a structure-based drug design method, and an RORγ inhibitor SR2211 can significantly inhibit expressions of the AR and AR-V7, and exhibit a good inhibitory effect on cells resistant to a second-generation drug enzalutamide. In addition, the RORγ inhibitor also inhibits tumors from growing in mouse xenograft models with CRPC. To conclude, an inhibition of the target RORγ can interfere with an expression of the AR gene and a downstream signaling pathway, thus providing a new treatment for prostate cancer and clinical drug resistance thereof.

### SUMMARY

In an aspect, the present application provides a benzo five-membered nitrogen heterocyclic compound. The benzo five-membered nitrogen heterocyclic compound is used as a compound of an RORγ receptor inhibitor. This type of compound can effectively inhibit RORγ proteins and is highly selective for other nuclear receptor family proteins.

The present application provides a benzo five-membered nitrogen heterocyclic compound. The benzo five-membered nitrogen heterocyclic compound has a structure represented by Formula I.

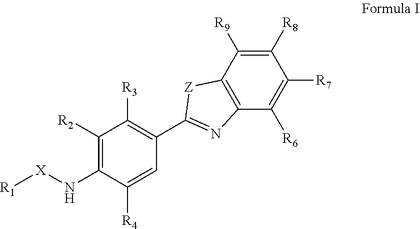

in Formula I, X is selected from

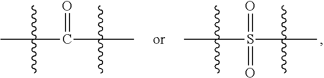

wherein the squiggle represents a bond of the group;
in Formula I, Y is selected from CR₅ or an N atom;
in Formula I, Z is selected from an S atom or NR₁₀;
in Formula I, R₁ and R₁₀ are each independently selected from any one of C1-C10 alkyl substituted with 0 to 3 R₁₁, C6-C10 aryl substituted with 0 to 3 R₁₁, C6-C10 aryl-C1-C3 alkyl substituted with 0 to 3 R₁₁, C2-C10 heteroaryl substituted with 0 to 3 R₁₁, a C2-C20 heterocyclyl substituted with 0 to 3 R₁₁, C2-C10 heteroaryl-C1-C3 alkyl substituted with 0 to 3 R₁₁, a C2-C20 heterocyclyl-C1-C3 alkyl substituted with 0 to 3 R₁₁, C3-C10 cycloalkyl substituted with 0 to 3 R₁₁ or C3-C10 cycloalkyl-C1-C3 alkyl substituted with 0 to 3 R₁₁;
R₁₁ is selected from any one of halogen, cyano, nitro, carboxyl, hydroxyl, amino, C1-C10 alkyl sulfonyl, C1-C10 alkyl sulfonyl substituted with at least one halogen, C1-C10 carbalkoxy, C1-C10 carbalkoxy substituted with at least one halogen, C1-C10 alkyl, C1-C10 alkyl substituted with at least one halogen, C1-C10 alkoxy, C1-C10 alkoxy substituted with at least one halogen, C3-C10 cycloalkyl or C3-C10 cycloalkyl substituted with at least one halogen; and
in Formula I, R₂ to R₉ are each independently selected from any one of hydrogen, halogen, C1-C10 alkyl, C1-C10 alkyl substituted with at least one halogen, C1-C10 alkoxy, C1-C10 alkoxy substituted with at least one halogen, C1-C10 alkylamide, C1-C10 alkylamide substituted with at least one halogen, C1-C10 cycloalkylamide, C1-C10 cycloalkylamide substituted with at least one halogen, C1-C10 alkylamino, C1-C10 alkylamino substituted with at least one halogen, C3-C10 cycloalkylamino or C3-C10 cycloalkylamino substituted with at least one halogen.

The term “substituted” means that one or more hydrogens on one or more designated atoms are substituted with a selection of an indicated group, provided that normal valences of the one or more designated atoms in an existing environment are not exceeded and the substitution results in a stable compound.

In the present application, halogen includes fluorine, chlorine, bromine and iodine.

In the present application, C2-C20 heterocyclyl refers to a monoheterocyclyl containing 2 to 10 C and a fused heterocyclyl containing 10 to 20 C. The monoheterocyclyl containing 2 to 10 C refers to a saturated or partially saturated and non-aromatic monocyclic cyclic group containing 1 to 4 heteroatoms (N, O or S). The fused heterocyclyl containing 10 to 20 C refers to a saturated or partially saturated and non-aromatic cyclic group containing 10 to 20 C atoms and 1 to 4 heteroatoms (N, O or S) and formed by two or more cyclic structures sharing two adjacent atoms with each other. The fused ring may have an aromatic ring, but the fused ring as a whole is not aromatic. Optionally, ring atoms (such as C, N or S) in the cyclic structure may be oxo. The mono-heterocyclyl containing 2 to 10 C includes, but is not limited to, 2H-aziridinyl, diaziridinyl, azetidinyl, 1,4-dioxanyl, 1,3-dioxolanyl, dihydropyrrolyl, pyrrolidinyl, imidazolidinyl, 4,5-dihydroimidazolyl, pyrazolidinyl, 4,5-dihydropyrazolyl, 2,5-dihydrothienyl, 4,5-dihydrothiazolyl, thiazolidinyl, piperidyl, tetrahydrothienyl, tetrahydrofuranyl, tetrahydropyridyl, piperidonyl, tetrahydropyridonyl, dihydropiperidonyl, piperazinyl and morpholinyl. The fused heterocyclyl containing 10 to 20 C includes, but is not limited to, benzopyrrolidinyl, benzocyclopentyl, benzocyclohexyl, benzotetrahydrofuranyl, benzopyrrolidinyl, benzimidazolidinyl, benzoxazolidinyl, benzothiazolidinyl, benzisoxazolidinyl, benzisothiazolidinyl, benzopiperidyl, benzomorpholinyl, benzopiperazinyl, benzotetrahydropyranyl, pyrrolidinocyclopropyl, cyclopentylazacyclopropyl, pyrrolidinocyclobutyl, pyrrolidinopyrrolidinyl, pyrrolidinopiperidyl, pyrrolidinopiperazinyl, pyrrolidinomorpholinyl, piperidomorpholinyl, pyridocyclopentyl, pyridocyclohexyl, pyridotetrahydrofuranyl, pyridopyrrolidinyl, pyridimidazolidinyl, pyridoxazolidinyl, pyridothiazolidinyl, pyridisoxazolidinyl, pyridisothiazolidinyl, pyridopiperidyl, pyridomorpholinyl, pyridopiperazinyl, pyridotetrahydropyranyl, pyrimidocyclopentyl, pyrimidocyclohexyl, pyrimidotetrahydrofuranyl, pyrimidopyrrolidinyl, pyrimidimidazolidinyl, pyrimidoxazolidinyl, pyrimidothiazolidinyl, pyrimidisoxazolidinyl, pyrimidisothiazolidinyl, pyrimidopiperidyl, pyrimidomorpholinyl, pyrimidopiperazinyl and pyrimidotetrahydropyranyl.

In the present application, C1-C10 alkyl refers to branched or unbranched chain alkyl having 1 to 10 carbon atoms, including but not limited to methyl, ethyl, n-propyl, isopropyl, n-butyl, tert-butyl, n-pentyl, n-hexyl and the like; and all the carbon atoms may be optionally substituted with one or more halogens.

In the present application, C1-C10 alkoxy refers to —O—C1-C10 alkyl, and C1-C10 alkyl is as defined above.

In the present application, C1-C10 alkylamide refers to —CO—NH—C1-C10 alkyl or C1-C10 alkyl-CO—NH—, and C1-C10 alkyl is as defined above.

In the present application, C3-C10 cycloalkyl refers to a saturated cyclic hydrocarbon having 3 to 10 carbon atoms, including but not limited to cyclobutyl, cyclopentyl, cyclohexyl and the like. All the carbon atoms of alkyl are optionally substituted with one or more halogens.

In the present application, C3-C10 cycloalkyl C1-C3 alkyl refers to C1-C3 cycloalkyl joined to C3-C10 alkyl, each of which has the same meaning as defined above, and C6-C10 aryl C1-C3 alkyl, C2-C10 heteroaryl C1-C3 alkyl and a C2-C20 heterocyclyl C1-C3 alkyl each have the same meaning and represent a group formed by joining two or three groups.

In the present application, C3-C10 cycloalkylamide refers to —CO—NH—C3-C10 cycloalkyl or C3-C10 cycloalkyl-CO—NH—, and C3-C10 cycloalkyl is as defined above.

In the present application, C2-C10 heteroaryl refers to a heteroaryl ring system containing 2 to 10 carbon atoms and 1 to 4 heteroatoms selected from N, O and S and containing aromatic 5- to 6-membered monocyclic heteroaryl and a bicyclic or fused ring (at least one of the rings is an aromatic ring) having aromatic 7- to 11-membered heteroaryl. 5- to 6-membered monocyclic heteroaryl includes, but is not limited to, pyridyl, imidazolyl, triazolyl, furanyl, oxazolyl, isoxazolyl, oxadiazolyl, pyrazinyl, pyridazinyl, pyrimidinyl, pyrrolyl and thienyl. The bicyclic or fused ring having 7- to 11-membered heteroaryl includes, but is not limited to, benzimidazolyl, quinolyl, isoquinolyl, quinazolinyl, indazolyl, thienopyrimidinyl, indolyl, isoindolyl, indazolyl, benzotriazolyl, benzofuranyl, benzopyranyl, benzoxazolyl, benzothiazolyl, pyrrolopyridyl and imidazopyridyl.

In the present application, C1-C10 alkyl sulfonyl refers to —SO₂—C1-C10 alkyl, and C1-C10 alkyl is as defined above.

In the present application, C1-C10 carbalkoxy refers to —COO—C1-C10 alkyl, and C1-C10 alkyl is as defined above.

In the present application, C1-C10 alkylamino refers to —NH—C1-C10 alkyl, and C1-C10 alkyl is as defined above.

Preferably, the C2-C20 heterocyclyl substituted with 0 to 3 R₁₁ is

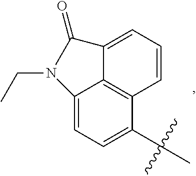 [SMILES: *c1ccc2c3c(cccc13)C(=O)N2CC]

wherein the squiggle represents a bond of the group.

Preferably, R₁₁ in R₁ is selected from any one or a combination of at least two of C1-C10 alkyl, halogen, trifluoromethoxy, nitro, cyano, carboxyl, methoxycarbonyl, ethoxycarbonyl, amino, methyl sulfonyl or ethyl sulfonyl, preferably, any one or a combination of at least two of cyano, ethoxycarbonyl or ethyl sulfonyl.

Preferably, each of R₂ to R₅ is hydrogen.

Preferably, R₆, R₇, R₈ and R₉ are not hydrogen at the same time.

Preferably, R₆ and R₉ are hydrogen.

Preferably, R₇ and R₈ are not hydrogen at the same time.

Preferably, one and only one of R₇ and R₈ is hydrogen.

Preferably, R₇ and R₈ are each independently selected from any one or a combination of at least two of hydrogen, methyl, methoxy, halogen, trifluoromethyl or C2-C10 alkylamide.

Preferably, C2-C10 alkylamide is isopropylamide or cyclopentylamide.

Preferably, R₁₀ is selected from any one of C1-C10 alkyl, cyclobutyl, cyclobutylmethyl, cyclohexylmethyl, pyridylmethyl, phenylethyl substituted with 0 to 3 R₁₁ or benzyl substituted with 0 to 3 R₁₁.

Preferably, R₁₁ in R₁₀ is selected from any one or a combination of at least two of methyl, carboxyl, trifluoromethyl, methoxycarbonyl, halogen, cyano or methyl sulfonyl.

Preferably, the benzo five-membered nitrogen heterocyclic compound has a structure represented by Formula II:

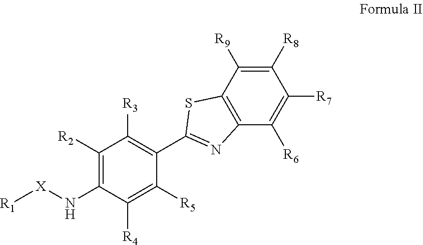

in Formula II, X is selected from

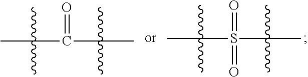

in Formula II, R₁ is selected from any one of C1-C10 alkyl substituted with 0 to 3 R₁₁, C6-C10 aryl substituted with 0 to 3 R₁₁, C6-C10 aryl C1-C3 alkyl substituted with 0 to 3 R₁₁ or a C2-C20 heterocyclyl (such as

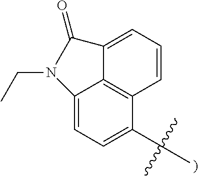 [SMILES: *c1ccc2c3c(cccc13)C(=O)N2CC]

substituted with 0 to 3 R₁₁; and R₁₁ is selected from any one of halogen, cyano, nitro, carboxyl, hydroxyl, amino, C1-C10 alkyl sulfonyl, C1-C10 alkyl sulfonyl substituted with at least one halogen, C1-C10 carbalkoxy, C1-C10 carbalkoxy substituted with at least one halogen, C1-C10 alkyl, C1-C10 alkyl substituted with at least one halogen, C1-C10 alkoxy or C1-C10 alkoxy substituted with at least one halogen;
in Formula II, R₂ to R₉ are each independently selected from any one of hydrogen, halogen, C1-C10 alkyl, C1-C10 alkyl substituted with at least one halogen, C1-C10 alkoxy or C1-C10 alkoxy substituted with at least one halogen.

Preferably, in Formula II, R₂ is selected from hydrogen or chlorine.

Preferably, in Formula II, R₈ is selected from any one of methyl, methoxy or fluorine.

Preferably, in Formula II, R₇ is hydrogen.

Preferably, the benzo five-membered nitrogen heterocyclic compound has a structure represented by Formula II-2:

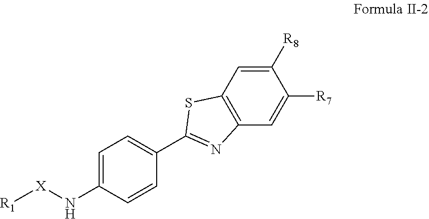

in Formula II-2, each of R₁, R₇ and R₈ is selected from the same range as that in Formula II.

Preferably, the benzo five-membered nitrogen heterocyclic compound has a structure represented by Formula III:

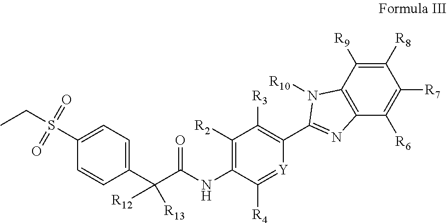

in Formula III, Y is selected from CR₅ or an N atom;
in Formula III, R₂ to R₉ are each independently selected from any one of hydrogen, halogen, C1-C10 alkyl, C1-C10 alkyl substituted with at least one halogen, C1-C10 alkoxy, C1-C10 alkoxy substituted with at least one halogen, C1-C10 alkylamide, C1-C10 alkylamide substituted with at least one halogen, C1-C10 cycloalkylamide or C1-C10 cycloalkylamide substituted with at least one halogen;
in Formula III, R₁₀ is selected from any one of C1-C10 alkyl substituted with 0 to 3 R₁₁, C6-C10 aryl-C1-C3 alkyl substituted with 0 to 3 R₁₁, C2-C10 heteroaryl-C1-C3 alkyl (such as pyridylmethylene) substituted with 0 to 3 R₁₁, C3-C10 cycloalkyl substituted with 0 to 3 R₁₁ or C3-C10 cycloalkyl-C1-C3 alkyl substituted with 0 to 3 R₁₁; and R₁₁ is selected from hydrogen, halogen, cyano, carboxyl, C1-C10 alkyl, C1-C10 alkyl substituted with at least one halogen, C1-C10 alkyl sulfonyl, C1-C10 alkyl sulfonyl substituted with at least one halogen, C1-C10 carbalkoxy or C1-C10 carbalkoxy substituted with at least one halogen;
in Formula III, R₁₂ and R₁₃ are each independently selected from hydrogen, amino, C1-C10 alkyl substituted with 0 to 3 R₁₄ or C1-C10 alkylamide substituted with 0 to 3 R₁₄, or R₁₂ and R₁₃ form a C3-C6 carbocyclic ring together with a carbon to which R₁₂ and R₁₃ are joined; and R₁₄ is selected from any one of halogen, carboxyl, hydroxyl, amino, C1-C10 alkylamide, C1-C10 alkylamino or C1-C10 carbalkoxy.

Preferably, R₇ is selected from any one of methyl, methoxy, isopropylamide or cyclopentylamide.

Preferably, R₈ is selected from any one of methyl, methoxy, trifluoromethyl, isopropylamide or cyclopentylamide.

Preferably, R₁₂ and R₁₃ are each independently selected from any one of hydrogen, amino or methylamide (CH₃—CO—NH—).

Preferably, the benzo five-membered nitrogen heterocyclic compound has a structure represented by Formula III-2:

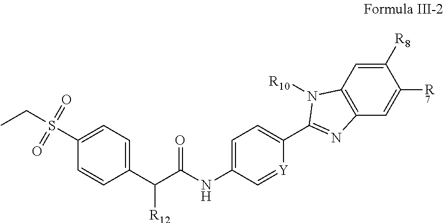

in Formula III, each of Y, R₇, R₈, R₁₀ and R₁₂ is selected from the same range as that in Formula III.

In a second aspect, the present application provides a pharmaceutically acceptable salt, isomer, racemate, prodrug, co-crystal complex or solvate of the benzo five-membered nitrogen heterocyclic compound according to the first aspect.

In the present application, the pharmaceutically acceptable salt of the present application may be synthesized by the compound of the present application containing a basic moiety or an acidic moiety through a conventional chemical method. Generally, a salt of a basic compound is prepared through a reaction of the basic compound and a suitable inorganic or organic acid in a suitable solvent or a combination of multiple solvents. Similarly, a salt of an acidic compound is formed through a reaction of the acidic compound and a suitable inorganic or organic base. Therefore, the pharmaceutically acceptable salt of the compound of the present application includes a conventional non-toxic salt of the compound of the present application formed through a reaction of a basic compound of the present application and an inorganic acid (such as hydrochloric acid, hydrobromic acid, sulfuric acid, sulfamic acid, phosphoric acid or nitric acid) or an organic acid (such as acetic acid, propanoic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamate, benzoic acid, salicylic acid, sulfanilic acid, 2-acetoxybenzoic acid, fumaric acid, toluenesulfonic acid, methanesulfonic acid, ethanedisulfonic acid, oxalic acid, isethionic acid or trifluoroacetic acid).

If the compound of the present application is acidic, the pharmaceutically acceptable salt of the compound of the present application includes a salt prepared by a pharmaceutically acceptable non-toxic base including an inorganic base (an aluminum salt, an ammonium salt, a calcium salt, a copper salt, an iron salt, a ferrous salt, a lithium salt, a magnesium salt, a manganese salt, a manganous salt, a potassium salt, a sodium salt and a zinc salt) and an organic base (salts of primary, secondary and tertiary amine).

The term “isomer” refers to compounds having the same chemical composition and different spatial arrangements of atoms or groups. Isomer mainly includes diastereomers and enantiomers.

The term “diastereomers” refers to stereoisomers that have two or more asymmetric centers and whose molecules are not mirror images of each other.

The term “enantiomers” refers to two stereoisomers of one compound that are non-superimposable mirror images of each other. An equimolar mixture of two enantiomers is referred to as a “racemic mixture” or “racemate”.

The term “prodrug” includes a compound having a moiety that may be metabolized in vivo. Typically, a prodrug is metabolized in vivo to an active drug by an esterase or other mechanisms. These prodrugs may be prepared in situ at the time of final isolation and purification of the compound, or the purified compound may be separately reacted in the form of an acid or hydroxyl with a suitable esterifying agent.

In a third aspect, the present application provides an application of the benzo five-membered nitrogen heterocyclic compound according to the first aspect or the pharmaceutically acceptable salt, isomer, racemate, prodrug, co-crystal complex or solvate according to the second aspect to preparation of an RORγ receptor inhibitor.

Preferably, the RORγ receptor inhibitor is used for preparing a drug for treating a cancer, a cell proliferative disorder, an inflammatory disease and an autoimmune disease, sepsis, a viral infection or a neurodegenerative disease.

Preferably, the RORγ receptor inhibitor is used for preparing a drug for treating a cancer.

Preferably, the RORγ receptor inhibitor is used for preparing a drug for treating prostate cancer.

In a fourth aspect, the present application provides a pharmaceutical composition. An active ingredient of the pharmaceutical composition includes the benzo five-membered nitrogen heterocyclic compound according to the first aspect or the pharmaceutically acceptable salt, isomer, racemate, prodrug, co-crystal complex or solvate according to the second aspect.

In a fifth aspect, the present application provides an application of the pharmaceutical composition according to the fourth aspect to preparation of a drug for treating, preventing or ameliorating an inflammation, an autoimmune disease, a cell proliferative disorder, sepsis, a cancer, a viral infection or a neurodegenerative disease.

The drug prepared by the RORγ receptor inhibitor and the pharmaceutical compositions can treat cancers such as adrenal tumor, acoustic neuroma, acral melanoma, acral hidradenoma, acute eosinophilic leukemia, acute erythroleukemia, acute lymphoblastic leukemia, acute megakaryocytic leukemia, acute monocytic leukemia, acute promyelocytic leukemia, adenocarcinoma, adenoid cystic carcinoma, adipose tissue tumor, adrenocortical carcinoma, adult T-cell leukemia/lymphoma, AIDS-related lymphoma, alveolar rhabdomyosarcoma, alveolar soft part sarcoma, ameloblastic fibroma, anaplastic large-cell lymphoma, undifferentiated thyroid cancer, angiomyolipoma, angiosarcoma, astrocytoma, atypical malformed rod-shaped tumor, B-cell chronic lymphocytic leukemia, B-cell prolymphocytic leukemia, B-cell lymphoma, basal cell carcinoma, biliary tract carcinoma, bladder cancer, blastoma, bone tumor, brown tumor, Burkitt's lymphoma, breast cancer, brain cancer, carcinoma in situ, chondroma, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colon cancer, small-round-cell tumor, diffuse cellular B-cell lymphoma, neuroepithelial tumor, dysgerminoma, embryonal carcinoma, endocrine tumor, endodermal sinus tumor, esophageal cancer, fibroma, fibrosarcoma, follicular lymphoma, follicular astrocytoma, thyroid cancer, gastrointestinal cancer, germ cell tumor, gestational choriocarcinoma, giant-cell fibroblastoma, giant-cell tumor of bone, gliocytoma, glioblastoma multiforme, glioma, granular cell tumor, arrhenoblastoma, gallbladder cancer, gastric cancer, hemangioblastoma, head and neck cancer, hemangiopericytoma malignant tumor, hepatoblastoma, cell lymphoma, Hodgkin's lymphoma, non-Hodgkin's lymphoma, invasive lobular carcinoma, intestinal cancer, kidney cancer, laryngeal cancer, fatal midline carcinoma, leukemia, Leydig cell tumor, liposarcoma, lung cancer, lymphangioma, lymphoepithelioma, lymphoma, acute lymphangiosarcoma, lymphocytic leukemia, chronic lymphocytic leukemia, liver cancer, small-cell lung cancer, non-small-cell lung cancer, MALT lymphoma, malignant fibrous histiocytoma, malignant peripheral schwannoma, marginal zone B-cell lymphoma, mast cell leukemia, mediastinal germ cell tumor, medullary breast carcinoma, medullary thyroid cancer, medulloblastoma, melanoma, meningioma, Merkel cell carcinoma, mesothelioma, metastatic cell carcinoma, mixed Müllerian tumor, mucinous tumor, multiple myeloma, muscle tissue tumor, muscarinic mucinous liposarcoma, myxoma, myxosarcoma, nasopharyngeal carcinoma, neuroblastoma, neurofibroma, neuroma, ocular neoplasm, acidophilia, optic nerve sheath meningioma, tumor, oral cancer, osteosarcoma, ovarian cancer, papillary thyroid cancer, tumor paraganglioma, pinealoblastoma, pituicytoma, precursor T-lymphoblastic lymphoma, primary central nervous system lymphoma, peritoneal cancer, prostate cancer, pancreatic cancer, pharyngeal cancer, renal cell carcinoma, renal medullary carcinoma, retinoblastoma, rhabdomyoma, rhabdomyosarcoma, rectal cancer, sarcoma, seminoma, trophoblastic tumor, skin cancer, small-round-cell tumor, small-cell carcinoma, soft tissue sarcoma, somatostatinoma, spinal tumor, splenic marginal zone lymphoma, squamous cell carcinoma, synovial sarcoma, small intestine cancer, squamous cell carcinoma, gastric cancer, T-cell lymphoma, testicular cancer, thyroid cancer, transitional cell cancer, laryngeal cancer, urachal cancer, urogenital cancer, uterine cancer, verrucous cancer, visual pathway glioma, vulvar cancer or vaginal cancer.

The drug prepared by the RORγ receptor inhibitor and the pharmaceutical composition can treat inflammatory diseases such as pelvic inflammatory disease, urethritis, skin sunburn, nasosinusitis, pneumonia, encephalitis, meningitis, myocarditis, nephritis, osteomyelitis, myositis, hepatitis, gastritis, enteritis, dermatitis, gingivitis, pancreatitis, psoriasis, allergy, Crohn's disease, bowel syndrome, ulcerative colitis, tissue transplant rejection, organ transplant rejection, asthma, allergic rhinitis, chronic obstructive pulmonary disease, autoimmune disease, autoimmune alopecia, anemia, glomerulonephritis, dermatomyositis, multiple sclerosis, scleroderma, vasculitis, autoimmune hemolysis and thrombocytopenia, goodpasture syndrome, atherosclerosis, Addison's disease, Parkinson's disease, Alzheimer's disease, diabetes, septic shock, systemic lupus erythematosus, rheumatoid arthritis, psoriatic arthritis, osteoarthritis, chronic idiopathic thrombocytopenic purpura, myasthenia gravis, Hashimoto's thyroiditis, allergic dermatitis, degenerative joint disease, Guillain-Barr\tilde{e} syndrome, mycosis fungoides or acute inflammatory response.

The drug prepared by the RORγ receptor inhibitor and the pharmaceutical composition can treat viral infections such as human papillomavirus infection, herpes virus infection, Epstein-Barr virus infection, human immunodeficiency virus infection, hepatitis B virus infection or hepatitis C virus infection.

The drug prepared by the RORγ receptor inhibitor and the pharmaceutical composition can treat neurodegenerative diseases such as Alzheimer's disease, amyotrophic lateral sclerosis, ataxia telangiectasia, bovine spongiform encephalopathy, Creutzfeldt-Jakob disease, Huntington's disease, cerebellar atrophy, multiple sclerosis, Parkinson's disease, primary lateral sclerosis or spinal muscular atrophy.

The drug prepared by the RORγ receptor inhibitor and the pharmaceutical composition may be suitable for a variety of administration routes which include typical but non-limiting examples such as oral administration, buccal administration, inhalation administration, sublingual administration, rectal administration, vaginal administration, intracisternal administration or intrathecal administration, administration through a lumbar puncture, transurethral administration, transdermal administration or parenteral administration (including intravenous injection, intramuscular injection, subcutaneous injection, intradermal injection, intraperitoneal injection, intrathecal injection and surgical implantation).

The pharmaceutical composition described in the present application may be in a liquid, semi-liquid or solid form and is prepared in a manner suitable for a used administration route. The composition described in the present application may be administered according to administration routes such as oral administration, parenteral administration, intraperitoneal administration, intravenous administration, transdermal administration, sublingual administration, intramuscular administration, rectal administration, buccal administration, intranasal administration or liposome.

An orally administered pharmaceutical composition may be a solid, a gel or a liquid. Examples of a solid preparation include, but are not limited to, a tablet, a capsule, a granule and a powder in bulk. These preparations may selectively contain an adhesive, a diluent, a disintegrant, a lubricant, a glidant, a sweetener, a corrigent or the like. Examples of the adhesive include, but are not limited to, microcrystalline cellulose, a glucose solution, acacia mucilage, a gelatin solution, sucrose and a starch paste. Examples of the lubricant include, but are not limited to, talcum, starch, magnesium stearate, calcium stearate and stearic acid. Examples of the diluent include, but are not limited to, lactose, sucrose, starch, mannitol and dicalcium phosphate. Examples of the glidant include, but are not limited to, silicon dioxide. Examples of the disintegrant include, but are not limited to, croscarmellose sodium, sodium starch glycolate, alginic acid, corn starch, potato starch, methyl cellulose, agar and carboxymethyl cellulose.

The pharmaceutical composition described in the present application is administered parenterally, generally through injection including subcutaneous, intramuscular or intravenous injection. An injectable may be prepared in any conventional form such as a liquid solution or a suspension, a solid form suitable for dissolution or suspension in a liquid before injection or an emulsion. Examples of a pharmaceutically acceptable carrier that may be used in the injectable of the present application include, but are not limited to, an aqueous carrier, a non-aqueous carrier, an antimicrobial, an isotonic agent, a buffering agent, an anti-oxidant, a suspending and dispersing agent, an emulsifier, a chelating agent and other pharmaceutically acceptable substances. Examples of the aqueous carrier include a sodium chloride injection, a Ringer's injection, an isotonic glucose injection, a sterile water injection, glucose and a lactated Ringer's injection. Examples of the non-aqueous carrier include plant-derived fixed oil, cottonseed oil, corn oil, sesame oil and peanut oil. Examples of the antimicrobial include m-cresol, benzyl alcohol, chlorobutanol, benzalkonium chloride and the like. Examples of the isotonic agent include sodium chloride and glucose. The buffering agent includes phosphate and citrate.

The pharmaceutical composition described in the present application may also be prepared into a sterile lyophilized powder injection. The compound is dissolved in a sodium phosphate buffer solution containing glucose or other suitable excipients, and then the solution is subjected to sterile filtration under standard conditions known to those skilled in the art followed by lyophilization to obtain the desired preparation.

Preferably, the cancer includes prostate cancer.

Compared with the existing art, the present application has the beneficial effects described below.

The present application provides one type of compound which has a new structure and may be used as the RORγ receptor inhibitor. This type of compound can effectively inhibit RORγ proteins and is highly selective for other nuclear receptor family proteins. The benzo five-membered nitrogen heterocyclic compound or the pharmaceutical composition thereof provided by the present application can be used for preparing the drug for treating, preventing or ameliorating diseases such as an inflammation, an autoimmune disease, a cell proliferative disorder, sepsis, a cancer, a neurodegenerative disease or a viral infection. The drug has a good inhibitory effect on a tumor and a prominent curative effect especially for prostate cancer with a tumor growth inhibition (TGI) up to 109%. The drug also has an improved effect on the treatment of other diseases and a broad application prospect. This type of compound is stable in structure, simple in synthesis method and suitable for large-scale industrial production.

### BRIEF DESCRIPTION OF DRAWINGS

FIG. 1 is a diagram illustrating tumor-inhibitory effects of Example 68 in Test Example 5 on 22Rv1 mouse xenograft models.

FIG. 2 is a diagram illustrating variations of weights of mice during an administration of Example 68 in Test Example 5.

### DETAILED DESCRIPTION

For a better understanding of the present application, examples of the present application are listed below. Those skilled in the art are to understand that examples described herein are merely used for a better understanding of the present application and are not to be construed as specific limitations to the present application.

The meanings represented by the English or English abbreviations involved in the following specific embodiments are as follows:

polyphosphoric acid (PPA); 2-(7-Azabenzotriazol-1-yl)-N,N,N′,N′-tetramethyluronium hexafluorophosphate (HATU); N,N-diisopropylethylamine (DIPEA); dichloromethane (DCM); 1-hydroxybenzotriazole (HOBT); 1-ethyl-3-(3-dimethylaminopropyl)carbodiimide hydrochloride (EDCI); and dimethylsulfoxide (DMSO).

The present application provides representative synthesis routes for the compounds of Formulas II and III. A benzothiazole compound has a structure represented by Formula II and preparation methods including Routes one and two; and a benzimidazole compound has a structure represent by Formula III and preparation methods including Routes three and four.

Route one is specifically as follows:

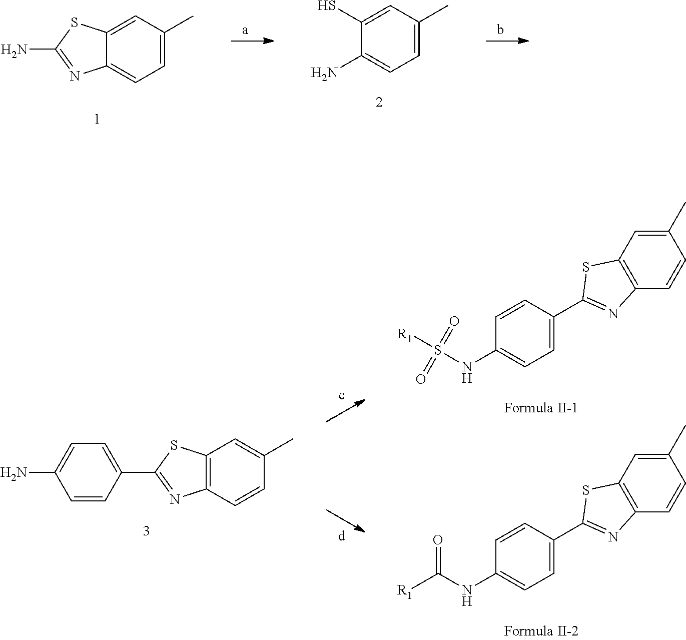

The reagents added in each step and the reaction conditions were as follows: (a) KOH, H₂O, refluxed, overnight; (b) PPA, 4-aminobenzoic acid or 4-amino-2-chlorobenzoic acid, 220° C., 4 h; (c) R₁SO₂Cl, pyridine, 80° C., overnight; (d) R₁COOH, HATU, DIPEA, DCM, room temperature, overnight; (e) NaOH, CH₃OH, room temperature, overnight.

Route two is specifically as follows:

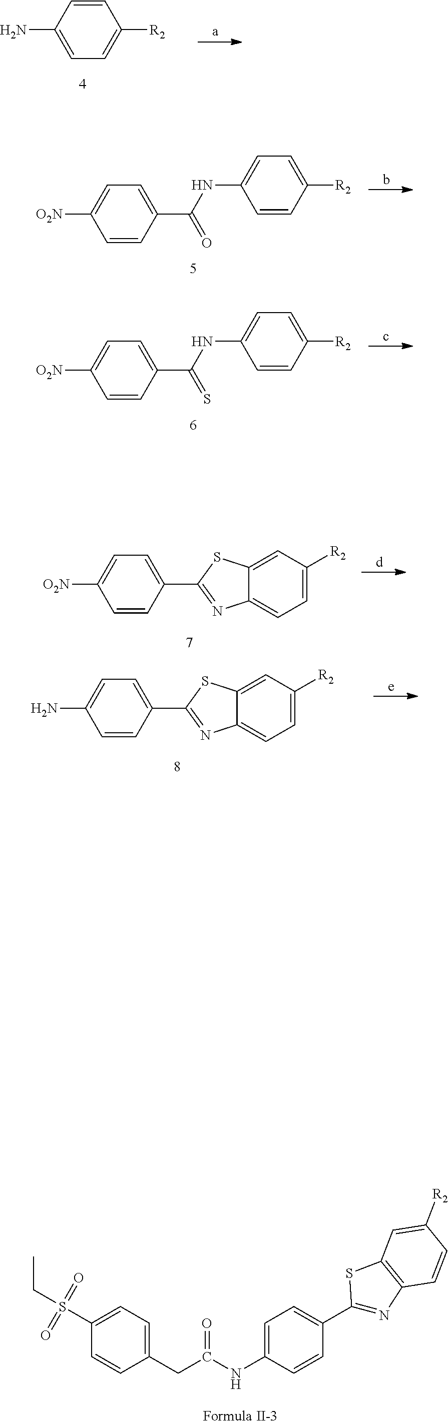

The reagent(s) added in each step and the reaction conditions were as follows: (a) pyridine, 40° C., 1 h; (b) Lawesson reagent, 1,4-dioxane, 110° C., 3 h; (c) NaOH, EtOH, potassium ferricyanide, 90° C., 30 min; (d) Pd/C, H₂, room temperature, 5 h; (e) HATU, DIPEA, DCM, room temperature, overnight.

Route three is specifically as follows:

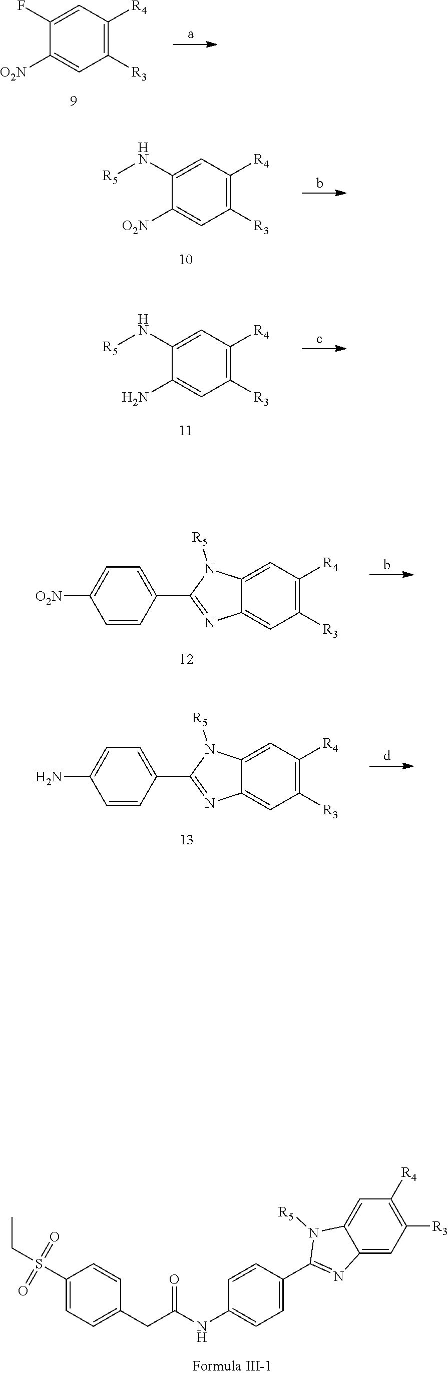

The reagents added in each step and the reaction conditions were as follows: (a) R₄NH₂, DIPEA, DMSO, 95° C., overnight; or R₄NH₂, K₂CO₃, DMF, 95° C., 1 h; (b) Fe, AcOH, NH₄Cl, H₂O, 80° C., 1 h; (c) 4-nitrobenzaldehyde, oxone, DMF, room temperature, 1 h; (d) 2-(4-(ethylsulfonyl)phenyl)acetic acid, HATU, DIPEA, DCM, room temperature, overnight; or 2-(4-(ethylsulfonyl)phenyl)acetic acid, HOBT, EDCI, DIPEA, DCM, room temperature, overnight; and (e) NaOH, CH₃OH, room temperature, overnight.

Route four is specifically as follows:

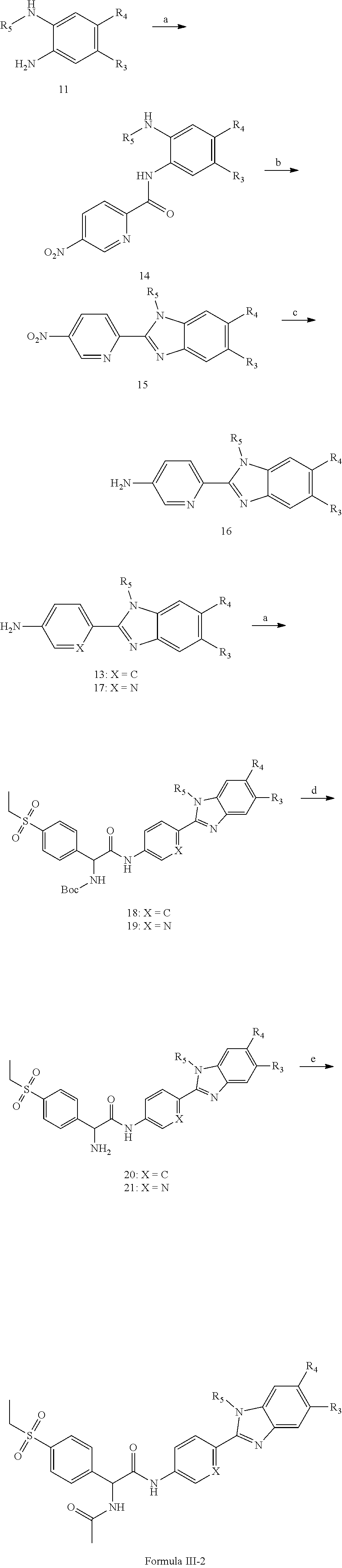

The reagent(s) added in each step and the reaction conditions were as follows: (a) 5-nitro-2-pyridinecarboxylic acid or 2-((tert-butoxycarbonyl)amino)-2-(4-(ethylsulfonyl)phenyl)acetic acid, HATU, DIPEA, DCM, room temperature, overnight; (b) acetic acid, 120° C., 4.5 h; (c) Fe, AcOH, ammonium chloride, H₂O, 80° C., 1 h; (d) trifluoroacetic acid (TFA), DCM, room temperature, 3 h; and (e) Ac₂O, DCM, Et₃N, room temperature, 3 h.

The above preparation methods are for illustrative purposes and not intended to limit the listed compounds or any particular substituent. The numbers of substituents shown in the embodiments do not necessarily correspond to the number used in the claims, and for the sake of clarity, it is shown that a single substituent is joined to a compound that allows for multiple substituents under the definitions of Formulas II and III above and all compounds contained by General Formula I can be obtained through variations in the numbers and substitution positions of the substituents.

### Example 1 4-methyl-N-(4-(6-methylbenzo[d]thiazol-2-yl)phenyl)benzenesulfonamide

### Step 1: synthesis of 2-amino-5-methylbenzenethiol

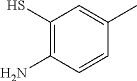

6-methylbenzo[d]thiazol-2-amine (5 g, 30.5 mmol) was suspended in a solution of KOH (25 g, 44.6 mmol) in water (50 mL) and heated to reflux overnight. The reaction was monitored through thin-layer chromatography (TLC). After the reaction was finished, the solution was cooled to ambient temperature and had a pH adjusted to 6 with acetic acid. The solution was filtered, and a thick precipitate was collected and rinsed with water. A residue was partitioned between dichloromethane and water and extracted. An organic layer was washed with brine, dried over anhydrous Na₂SO₄ and concentrated in vacuum to obtain a target compound as a yellow solid (3.4 g with a yield of 80%). ¹H NMR (400 MHz, DMSO-d₆) δ 6.91 (dd, J=8.2, 1.7 Hz, 1H), 6.81 (d, J=1.4 Hz, 1H), 6.64 (d, J=8.2 Hz, 1H), 5.21 (s, 2H), 2.05 (s, 3H).

### Step 2: synthesis of 4-(6-methylbenzo[d]thiazol-2-yl) aniline

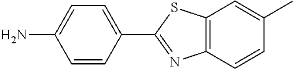 [SMILES: Cc1ccc2nc(-c3ccc(N)cc3)sc2c1]

Polyphosphoric acid (20 g) was added to a mixture of 2-amino-5-methylbenzenethiol (2.03 g, 14.58 mmol) and 4-aminobenzoic acid (1.99 g, 14.51 mmol) and heated for 4 h at 220° C. The reaction was monitored through the TLC. After the reaction was finished, the reaction mixture was cooled to ambient temperature, slowly poured into an ice-cold aqueous solution of sodium carbonate (10% w/v) and stirred until a gas ceased to escape. The solution was filtered, and a precipitate was collected and washed with water. A residue was partitioned between ethyl acetate and water and extracted. An organic layer was washed with brine, dried over anhydrous sodium sulfate and concentrated in vacuum to obtain a crude product. The crude product was purified through silica gel column chromatography (PE:EA=4:1, v/v) to obtain a target compound as a brown solid (3.14 g with a yield of 90%). ¹H NMR (400 MHz, DMSO-d₆) δ 7.80 (s, 1H), 7.77 (d, J=8.3 Hz, 1H), 7.72 (d, J=8.6 Hz, 2H), 7.26 (dd, J=8.3, 1.1 Hz, 1H), 6.65 (d, J=8.6 Hz, 2H), 5.85 (s, 2H), 2.42 (s, 3H).

### Step 3: synthesis of 4-methyl-N-(4-(6-methylbenzo[d]thiazol-2-yl)phenyl)benzenesulfonamide (4a)

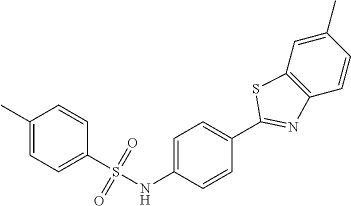 [SMILES: Cc1ccc(S(=O)(=O)Nc2ccc(-c3nc4ccc(C)cc4s3)cc2)cc1]

The compounds 4-(6-methylbenzo[d]thiazol-2-yl) aniline (73 mg, 0.3 mmol) and 4-toluenesulfonyl chloride (85.8 mg, 0.45 mmol) were dissolved in pyridine (10 mL) and reacted for 4 h at 80° C. The reaction was monitored through the TLC. After the reaction was finished, the reaction mixture was cooled to room temperature. Dilute hydrochloric acid (30 mL) was added to the reaction mixture and extracted three times (50 mL×3) with ethyl acetate. The organic phases were combined, washed once with saturated brine, dried over anhydrous sodium sulfate, concentrated under reduced pressure and isolated through the silica gel column chromatography (PE:EA=4:1, v/v) to obtain a target compound as a white solid (37 mg with a yield of 31%). ¹H NMR (500 MHz, DMSO-d₆) δ 10.71 (s, 1H), 7.93 (d, J=8.7 Hz, 2H), 7.90-7.85 (m, 2H), 7.72 (d, J=8.3 Hz, 2H), 7.37 (d, J=8.2 Hz, 2H), 7.32 (dd, J=8.4, 1.1 Hz, 1H), 7.27 (d, J=8.7 Hz, 2H), 2.43 (s, 3H), 2.32 (s, 3H). MS (ESI), m/z for C₂₁H₁₈N₂O₂S₂ ([M+H]⁺): Calcd 394.51, found 395.0.

### Example 2 4-(tert-butyl)-N-(4-(6-methylbenzo[d]thiazol-2-yl)phenyl)benzenesulfonamide

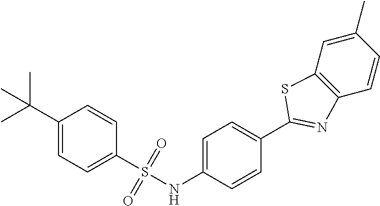 [SMILES: Cc1ccc2nc(-c3ccc(NS(=O)(=O)c4ccc(C(C)(C)C)cc4)cc3)sc2c1]

A synthesis method was as that in Example 1, and a white solid was obtained with a yield of 79%. ¹H NMR (400 MHz, DMSO-d₆) δ 10.75 (s, 1H), 7.94 (d, J=8.7 Hz, 2H), 7.90-7.84 (m, 2H), 7.78 (d, J=8.5 Hz, 2H), 7.60 (d, J=8.6 Hz, 2H), 7.35-7.26 (m, 3H), 2.44 (s, 3H), 1.25 (s, 9H). MS (ESI), m/z for C₂₄H₂₄N₂O₂S₂ ([M+H]⁺): Calcd 436.59, found 437.2.

### Example 3 4-fluoro-N-(4-(6-methylbenzo[d]thiazol-2-yl)phenyl)benzenesulfonamide

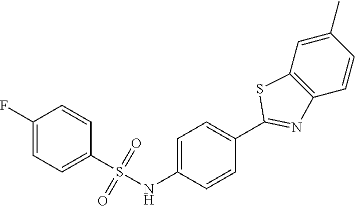 [SMILES: Cc1ccc2nc(-c3ccc(NS(=O)(=O)c4ccc(F)cc4)cc3)sc2c1]

A synthesis method was as that in Example 1, and a white solid was obtained with a yield of 61%. ¹H NMR (400 MHz, DMSO-d₆) δ 10.76 (s, 1H), 7.95 (d, J=8.7 Hz, 2H), 7.92-7.86 (m, 4H), 7.42 (t, J=8.8 Hz, 2H), 7.33 (dd, 8.4, 0.8 Hz, 1H), 7.28 (d, J=8.7 Hz, 2H), 2.44 (s, 3H). MS (ESI), m/z for C₂₀H₁₅FN₂O₂S₂ ([M+H]⁺): Calcd 398.47, found 398.9.

### Example 4 N-(4-(6-methylbenzo[d]thiazol-2-yl)phenyl)-4-(trifluoromethoxy) benzenesulfonamide

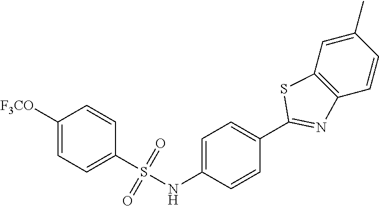 [SMILES: Cc1ccc2nc(-c3ccc(NS(=O)(=O)c4ccc(OC(F)(F)F)cc4)cc3)sc2c1]

A synthesis method was as that in Example 1, and a white solid was obtained with a yield of 74%. ¹H NMR (400 MHz, DMSO-d₆) δ 10.87 (s, 1H), 8.00-7.92 (m, 4H), 7.91-7.85 (m, 2H), 7.58 (d, J=8.3 Hz, 2H), 7.33 (d, J=8.5 Hz, 1H), 7.29 (d, J=8.7 Hz, 2H), 2.44 (s, 3H). MS (ESI), m/z for C₂₁H₁₅F₃N₂O₃S₂ ([M+H]⁺): Calcd 464.48, found 465.0.

### Example 5 N-(4-(6-methylbenzo[d]thiazol-2-yl)phenyl)-4-nitrobenzenesulfonamide

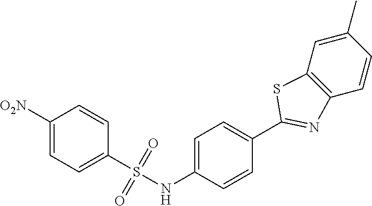 [SMILES: Cc1ccc2nc(-c3ccc(NS(=O)(=O)c4ccc([N+](=O)[O-])cc4)cc3)sc2c1]

A synthesis method was as that in Example 1, and a white solid was obtained with a yield of 50%. ¹H NMR (400 MHz, DMSO-d₆) δ 11.04 (s, 1H), 8.39 (d, J=8.9 Hz, 2H), 8.07 (d, J=8.9 Hz, 2H), 7.97 (d, J=8.7 Hz, 2H), 7.91-7.85 (m, 2H), 7.36-7.27 (m, 3H), 2.44 (s, 3H). MS (ESI), m/z for C₂₀H₁₅N₃O₄S₂ ([M+H]⁺): Calcd 425.48, found 426.0.

### Example 6 N-(4-(6-methylbenzo[d]thiazol-2-yl)phenyl)-3-nitrobenzenesulfonamide

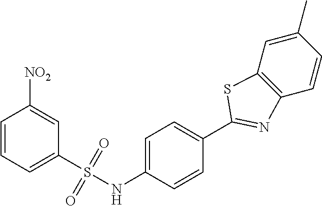 [SMILES: Cc1ccc2nc(-c3ccc(NS(=O)(=O)c4cccc([N+](=O)[O-])c4)cc3)sc2c1]

A synthesis method was as that in Example 1, and a white solid was obtained with a yield of 38%. ¹H NMR (400 MHz, DMSO-d₆) δ 11.01 (s, 1H), 8.58-8.54 (m, 1H), 8.46 (dd, J=8.2, 1.6 Hz, 1H), 8.21 (d, J=7.9 Hz, 1H), 7.96 (d, J=8.6 Hz, 2H), 7.91-7.85 (m, 3H), 7.36-7.27 (m, 3H), 2.44 (s, 3H). MS (ESI), m/z for C₂₀H₁₅N₃O₄S₂ ([M+H]⁺): Calcd 425.48, found 426.0.

### Example 7 N-(4-(6-methylbenzo[d]thiazol-2-yl)phenyl)-2-nitrobenzenesulfonamide

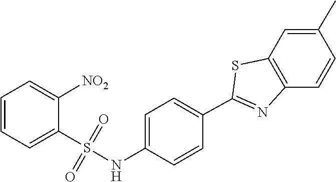 [SMILES: Cc1ccc2nc(-c3ccc(NS(=O)(=O)c4ccccc4[N+](=O)[O-])cc3)sc2c1]

A synthesis method was as that in Example 1, and a white solid was obtained with a yield of 54%. ¹H NMR (400 MHz, DMSO-d₆) δ 11.16 (s, 1H), 8.07-8.03 (m, 1H), 8.03-7.95 (m, 3H), 7.91-7.81 (m, 4H), 7.36-7.32 (m, 1H), 7.30 (d, J=8.7 Hz, 2H), 2.44 (s, 3H). MS (ESI), m/z for C₂₀H₁₅N₃O₄S₂ ([M+H]⁺): Calcd 425.48, found 426.0.

### Example 8 N-(4-(6-methylbenzo[d]thiazol-2-yl)phenyl)-3-(methanesulfonyl) benzenesulfonamide

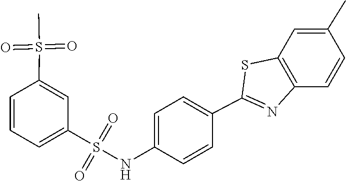 [SMILES: Cc1ccc2nc(-c3ccc(NS(=O)(=O)c4cccc(S(C)(=O)=O)c4)cc3)sc2c1]

A synthesis method was as that in Example 1, and a white solid was obtained with a yield of 34%. ¹H NMR (400 MHz, DMSO-d₆) δ 10.93 (s, 1H), 8.32 (s, 1H), 8.20 (d, J=7.7 Hz, 1H), 8.12 (d, J=8.0 Hz, 1H), 7.96 (d, J=8.6 Hz, 2H), 7.92-7.84 (m, 3H), 7.33 (d, J=8.8 Hz, 1H), 7.29 (d, J=8.6 Hz, 2H), 3.28 (s, 3H), 2.44 (s, 3H). MS (ESI), m/z for C₂₁H₁₈N₂O₄S₃ ([M+H]⁺): Calcd 458.57, found 459.2.

### Example 9 2,4-difluoro-N-(4-(6-methylbenzo[d]thiazol-2-yl)phenyl)benzenesulfonamide

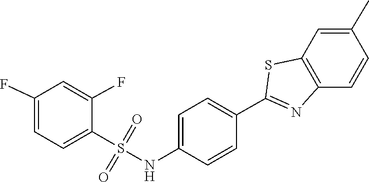 [SMILES: Cc1ccc2nc(-c3ccc(NS(=O)(=O)c4ccc(F)cc4F)cc3)sc2c1]

A synthesis method was as that in Example 1, and a white solid was obtained with a yield of 73%. ¹H NMR (400 MHz, DMSO-d₆) δ 11.13 (s, 1H), 8.03-7.92 (m, 3H), 7.90-7.85 (m, 2H), 7.58-7.50 (m, 1H), 7.36-7.24 (m, 4H), 2.44 (s, 3H). MS (ESI), m/z for C₂₀H₁₄F₂N₂O₂S₂ ([M+H]⁺): Calcd 416.46, found 417.1.

### Example 10 2,4,6-trimethyl-N-(4-(6-methylbenzo[d]thiazol-2-yl)phenyl)benzenesulfonamide

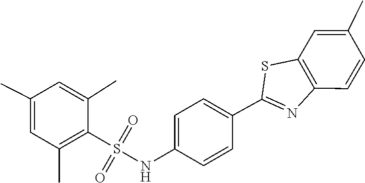 [SMILES: Cc1cc(C)c(S(=O)(=O)Nc2ccc(-c3nc4ccc(C)cc4s3)cc2)c(C)c1]

A synthesis method was as that in Example 1, and a white solid was obtained with a yield of 72%. ¹H NMR (400 MHz, DMSO-d₆) δ 10.69 (s, 1H), 7.92 (d, J=8.7 Hz, 2H), 7.89-7.83 (m, 2H), 7.32 (dd, J=8.4, 0.8 Hz, 1H), 7.13 (d, J=8.7 Hz, 2H), 7.03 (s, 2H), 2.61 (s, 6H), 2.43 (s, 3H), 2.21 (s, 3H). MS (ESI), m/z for C₂₃H₂₂N₂O₂S₂ ([M−H]⁻): Calcd 422.56, found 421.2.

### Example 11 1-ethyl-N-(4-(6-methylbenzo[d]thiazol-2-yl)phenyl)-2-oxo-1,2-dihydrobenzo[cd]indol-6-sulfonamide

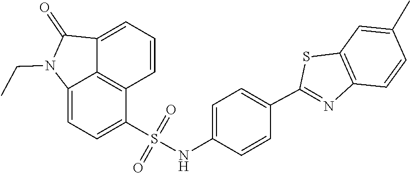 [SMILES: CCN1C(=O)c2cccc3c(S(=O)(=O)Nc4ccc(-c5nc6ccc(C)cc6s5)cc4)ccc1c23]

A synthesis method was as that in Example 1, and a white solid was obtained with a yield of 19%. ¹H NMR (500 MHz, DMSO-d₆) δ 11.06 (s, 1H), 8.74 (d, J=8.4 Hz, 1H), 8.26 (d, J=7.7 Hz, 1H), 8.13 (d, J=7.0 Hz, 1H), 8.00-7.93 (m, 1H), 7.90-7.81 (m, 4H), 7.32-7.28 (m, 2H), 7.24 (d, J=8.8 Hz, 2H), 3.88 (q, J=7.1 Hz, 2H), 2.42 (s, 3H), 1.22 (t, J=7.2 Hz, 3H). MS (ESI), m/z for C₂₇H₂₁N₃O₃S₂ ([M+H]⁺): Calcd 499.60, found 500.02.

### Example 12 N-(4-(6-methylbenzo[d]thiazol-2-yl)phenyl)-1-(p-tolyl)methanesulfonamide

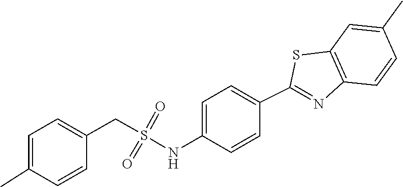

A synthesis method was as that in Example 1, and a white solid was obtained with a yield of 31%. ¹H NMR (400 MHz, DMSO) δ 10.22 (s, 1H), 8.01 (d, J=8.7 Hz, 2H), 7.94-7.87 (m, 2H), 7.38-7.30 (m, 3H), 7.20-7.09 (m, 4H), 4.51 (s, 2H), 2.46 (s, 3H), 2.28 (s, 3H). MS (ESI), m/z for C₂₂H₂₀N₂O₂S₂ ([M+H]⁺): Calcd 408.53, found 409.0.

### Example 13 1-(4-fluorophenyl)-N-(4-(6-methylbenzo[d]thiazol-2-yl)phenyl)methane sulfonamide

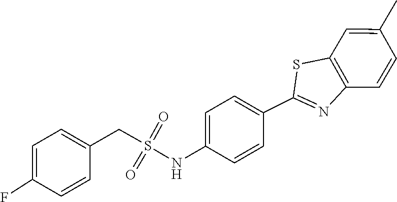 [SMILES: Cc1ccc2nc(-c3ccc(NS(=O)(=O)Cc4ccc(F)cc4)cc3)sc2c1]

A synthesis method was as that in Example 1, and a white solid was obtained with a yield of 26%. ¹H NMR (400 MHz, DMSO-d₆) δ 10.26 (s, 1H), 8.01 (d, J=8.7 Hz, 2H), 7.94-7.87 (m, 2H), 7.38-7.27 (m, 5H), 7.19 (t, J=8.9 Hz, 2H), 4.60 (s, 2H), 2.46 (s, 3H). MS (ESI), m/z for C₂₁H₁₇FN₂O₂S₂ ([M−1]⁻): Calcd 412.50, found 411.1.

### Example 14 N-(4-(6-methylbenzo[d]thiazol-2-yl)phenyl)-1-(4-(trifluoromethyl)phenyl)methane sulfonamide

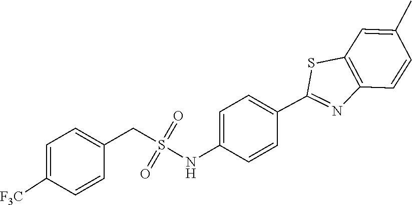 [SMILES: Cc1ccc2nc(-c3ccc(NS(=O)(=O)Cc4ccc(C(F)(F)F)cc4)cc3)sc2c1]

A synthesis method was as that in Example 1, and a white solid was obtained with a yield of 47%. ¹H NMR (500 MHz, DMSO-d₆) δ 10.34 (s, 1H), 8.01 (d, J=8.7 Hz, 2H), 7.94-7.88 (m, 2H), 7.74 (d, J=8.2 Hz, 2H), 7.51 (d, J=8.1 Hz, 2H), 7.38-7.30 (m, 3H), 4.75 (s, 2H), 2.46 (s, 3H). MS (ESI), m/z for C₂₂H₁₇F₃N₂O₂S₂ ([M+H]⁺): Calcd 462.51, found 463.0.

### Example 15 N-(4-(6-methylbenzo[d]thiazol-2-yl)phenyl)-1-(4-nitrophenyl)methanesulfonamide

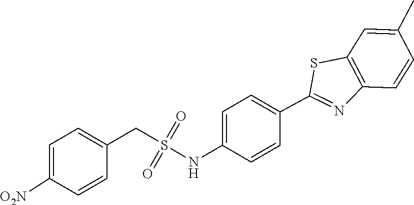 [SMILES: Cc1ccc2nc(-c3ccc(NS(=O)(=O)Cc4ccc([N+](=O)[O-])cc4)cc3)sc2c1]

A synthesis method was as that in Example 1, and a white solid was obtained with a yield of 40%. ¹H NMR (400 MHz, DMSO-d₆) δ 10.36 (s, 1H), 8.22 (d, J=8.7 Hz, 2H), 8.02 (d, J=8.6 Hz, 2H), 7.94-7.88 (m, 2H), 7.57 (d, J=8.7 Hz, 2H), 7.38-7.31 (m, 3H), 4.81 (s, 2H), 2.46 (s, 3H). MS (ESI), m/z for C₂₁H₁₇N₃O₄S₂ ([M+H]⁺): Calcd 439.50, found 440.0.

### Example 16 1-(4-cyanophenyl)-N-(4-(6-methylbenzo[d]thiazol-2-yl)phenyl)methanesulfonamide

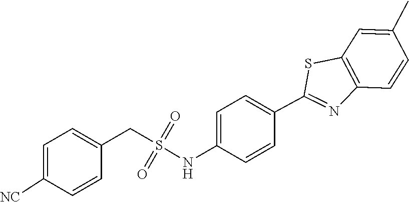 [SMILES: Cc1ccc2nc(-c3ccc(NS(=O)(=O)Cc4ccc(C#N)cc4)cc3)sc2c1]

A synthesis method was as that in Example 1, and a white solid was obtained with a yield of 22%. ¹H NMR (500 MHz, DMSO-d₆) δ 10.36 (s, 1H), 8.02 (d, J=8.7 Hz, 2H), 7.94-7.88 (m, 2H), 7.85 (d, J=8.2 Hz, 2H), 7.48 (d, J=8.2 Hz, 2H), 7.38-7.30 (m, 3H), 4.75 (s, 2H), 2.46 (s, 3H). MS (ESI), m/z for C₂₂H₁₇N₃O₂S₂ ([M+H]⁺): Calcd 419.52, found 420.0.

### Example 17 N-(4-(6-methylbenzo[d]thiazol-2-yl)phenyl)-1-(4-(methylsulfonyl)phenyl)methane sulfonamide

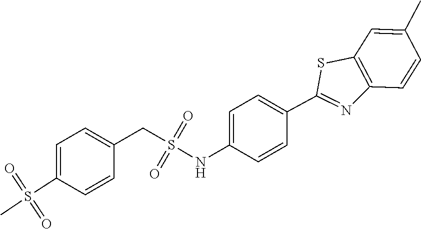

A synthesis method was as that in Example 1, and a white solid was obtained with a yield of 60%. ¹H NMR (400 MHz, DMSO-d₆) δ 10.37 (s, 1H), 8.02 (d, J=8.7 Hz, 2H), 7.94-7.88 (m, 4H), 7.56 (d, J=8.3 Hz, 2H), 7.39-7.30 (m, 3H), 4.76 (s, 2H), 3.20 (s, 3H), 2.46 (s, 3H). MS (ESI), m/z for C₂₂H₂₀N₂O₄S₃ ([M+H]⁺): Calcd 472.59, found 472.9.

### Example 18 4-((N-(4-(6-methylbenzo[d]thiazol-2-yl)phenyl)sulfamoyl)methyl)methyl benzoate

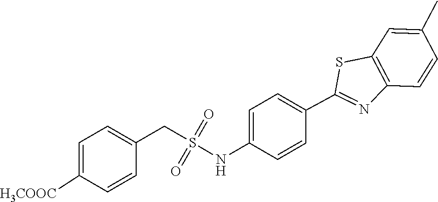

A synthesis method was as that in Example 1, and a white solid was obtained with a yield of 30%. ¹H NMR (500 MHz, DMSO-d₆) δ 10.33 (s, 1H), 8.01 (d, J=8.6 Hz, 2H), 7.95-7.89 (m, 4H), 7.43 (d, J=8.2 Hz, 2H), 7.37-7.30 (m, 3H), 4.71 (s, 2H), 3.83 (s, 3H), 2.46 (s, 3H). MS (ESI), m/z for C₂₃H₂₀N₂O₄S₂ ([M+H]⁺): Calcd 452.54, found 453.0.

### Example 19 4-((N-(4-(6-methylbenzo[d]thiazol-2-yl)phenyl)sulfamoyl)methyl)benzoic acid

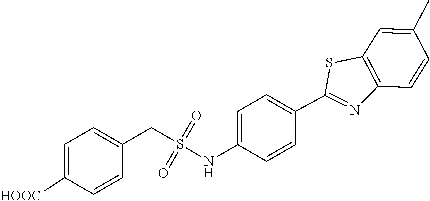 [SMILES: Cc1ccc2nc(-c3ccc(NS(=O)(=O)Cc4ccc(C(=O)O)cc4)cc3)sc2c1]

Example 18 (40 mg, 0.09 mmol) was dissolved in methanol (5 mL), and NaOH (10 mL, 2 M) was added to the mixture and stirred for 2 h at room temperature. After the reaction was finished, methanol was removed under reduced pressure, and the mixture had a pH adjusted to 5 to 6 with dilute hydrochloric acid (1 M). A precipitated solid was suction filtered to obtain a target compound as a white solid (28.8 mg with a yield of 73%). ¹H NMR (500 MHz, DMSO-d₆) δ 10.38 (s, 1H), 8.23 (d, J=8.7 Hz, 2H), 8.02 (d, J=8.7 Hz, 2H), 7.94-7.88 (m, 2H), 7.57 (d, J=8.6 Hz, 2H), 7.38-7.31 (m, 3H), 4.82 (s, 2H), 2.46 (s, 3H). MS (ESI), m/z for C₂₂H₁₈N₂O₄S₂ ([M+H]⁺): Calcd 438.52, found 440.0. MS (ESI), m/z for C₂₃H₂₀N₂O₄S₂ ([M+H]⁺): Calcd 452.54, found 453.0.

### Example 20 4-((N-(4-(6-methylbenzo[d]thiazol-2-yl)phenyl)sulfamoyl)methyl)phenyl propionate

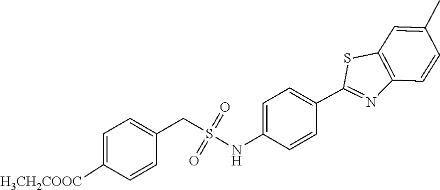

A synthesis method was as that in Example 1, and a white solid was obtained with a yield of 55%. ¹H NMR (400 MHz, DMSO-d₆) δ 10.30 (s, 1H), 8.01 (d, J=8.7 Hz, 2H), 7.95-7.87 (m, 4H), 7.42 (d, J=8.2 Hz, 2H), 7.38-7.29 (m, 3H), 4.70 (s, 2H), 4.29 (q, J=7.1 Hz, 2H), 2.46 (s, 3H), 1.28 (t, J=7.1 Hz, 3H). MS (ESI), m/z for C₂₄H₂₂N₂O₄S₂ ([M+H]⁺): Calcd 466.57, found 467.1.

### Example 21 N-(4-(6-methylbenzo[d]thiazol-2-yl)phenyl)-1-(3-nitrophenyl)methanesulfonamide

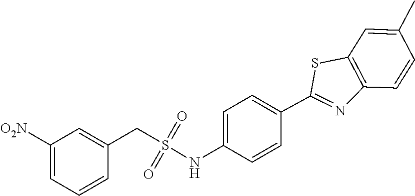 [SMILES: Cc1ccc2nc(-c3ccc(NS(=O)(=O)Cc4cccc([N+](=O)[O-])c4)cc3)sc2c1]

A synthesis method was as that in Example 1, and a white solid was obtained with a yield of 55%. ¹H NMR (400 MHz, DMSO-d₆) δ 10.33 (s, 1H), 8.21 (d, J=8.2 Hz, 1H), 8.16 (s, 1H), 8.00 (d, J=8.7 Hz, 2H), 7.94-7.88 (m, 2H), 7.74 (d, J=7.7 Hz, 1H), 7.67 (t, J=7.8 Hz, 1H), 7.38-7.29 (m, 3H), 4.83 (s, 2H), 2.46 (s, 3H). MS (ESI), m/z for C₂₁H₁₇N₃O₄S₂ ([M−H]⁻): Calcd 439.50, found 438.0.

### Example 22 N-(4-(6-methylbenzo[d]thiazol-2-yl)phenyl)-1-(2-nitrophenyl)methanesulfonamide

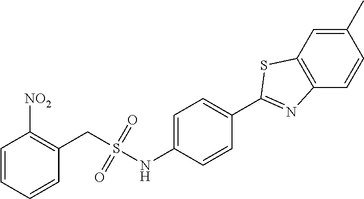 [SMILES: Cc1ccc2nc(-c3ccc(NS(=O)(=O)Cc4ccccc4[N+](=O)[O-])cc3)sc2c1]

A synthesis method was as that in Example 1, and a white solid was obtained with a yield of 20%. ¹H NMR (400 MHz, DMSO-d₆) δ 10.47 (s, 1H), 8.04 (d, J=8.1 Hz, 1H), 8.00 (d, J=8.6 Hz, 2H), 7.94-7.89 (m, 2H), 7.73 (td, J=7.6, 0.7 Hz, 1H), 7.64 (t, J=7.5, Hz, 1H), 7.50 (d, J=7.0 Hz, 1H), 7.35 (dd, J=8.5, 0.8 Hz, 1H), 7.28 (d, J=8.7 Hz, 2H), 5.05 (s, 2H), 2.46 (s, 3H). MS (ESI), m/z for C₂₁H₁₇N₃O₄S₂ ([M+H]⁺): Calcd 439.50, found 440.0.

### Example 23 3-((N-(4-(6-methylbenzo[d]thiazol-2-yl)phenyl)sulfamoyl)methyl)methyl benzoate

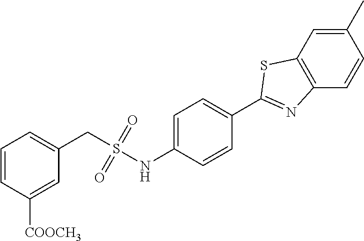 [SMILES: COC(=O)c1cccc(CS(=O)(=O)Nc2ccc(-c3nc4ccc(C)cc4s3)cc2)c1]

A synthesis method was as that in Example 1, and a white solid was obtained with a yield of 25%. ¹H NMR (400 MHz, DMSO-d₆) δ 10.27 (s, 1H), 8.00 (d, J=8.6 Hz, 2H), 7.95-7.86 (m, 4H), 7.58-7.48 (m, 2H), 7.37-7.28 (m, 3H), 4.71 (s, 2H), 3.83 (s, 3H), 2.46 (s, 3H). MS (ESI), m/z for C₂₃H₂₀N₂O₄S₂ ([M+H]⁺): Calcd 452.54, found 453.0.

### Example 24 1-(3-aminophenyl)-N-(4-(6-methylbenzo[d]thiazol-2-yl)phenyl)methane sulfonamide

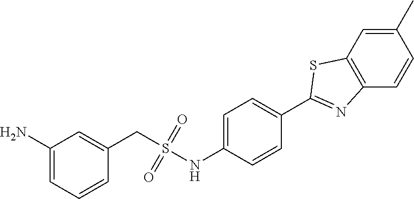 [SMILES: Cc1ccc2nc(-c3ccc(NS(=O)(=O)Cc4cccc(N)c4)cc3)sc2c1]

Example 21 (50 mg, 0.11 mmol) and 10% palladium on carbon (about 55% water content) (100 mg) were added to a solvent of MeOH (10 mL) and stirred overnight at room temperature in a hydrogen environment. After the reaction was finished, the mixture was suction filtered with Celite as a filter aid, and a filtrate was concentrated to obtain a crude product. The crude product was isolated through silica gel column chromatography (PE:EA=4:1, v/v) to obtain a target compound as a white solid (13 mg with a yield of 29%). ¹H NMR (400 MHz, DMSO-d₆) δ 10.24 (s, 1H), 8.15-7.78 (m, 4H), 7.47-7.21 (m, 3H), 7.09-6.85 (m, 1H), 6.62-6.45 (m, 2H), 6.44-6.25 (m, 1H), 5.11 (s, 2H), 4.35 (s, 2H), 2.45 (s, 3H). MS (ESI), m/z for C₂₁H₁₉N₃O₂S₂ ([M+H]⁺): Calcd 409.52, found 410.0.

### Example 25 2-(4-(Ethylsulfonyl)phenyl)-N-(4-(6-methylbenzo[d]thiazol-2-yl)phenyl)acetamide

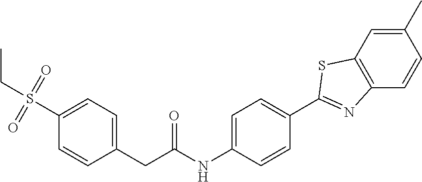 [SMILES: CCS(=O)(=O)c1ccc(CC(=O)Nc2ccc(-c3nc4ccc(C)cc4s3)cc2)cc1]

The compounds 4-(6-methylbenzo[d]thiazol-2-yl)aniline (80 mg, 0.33 mmol), diisopropylethylamine (127.7 mg, 0.99 mmol) and HATU (188.1 mg, 0.495 mmol) were dissolved in DCM (10 mL). The reaction mixture was stirred for 15 min, then the compound 2-(4-(ethylsulfonyl)phenyl)acetic acid (112.86 mg, 0.495 mmol) was added to the reaction mixture, and the obtained mixture was stirred overnight at room temperature. After the reaction was finished, water was added to dilute the mixture and extracted with DCM (20 mL×3). Combined organic layers were dried over anhydrous Na₂SO₄ and concentrated under reduced pressure. A crude product was isolated through silica gel column chromatography (PE:EA=3:1, v/v) to obtain a target compound as a white solid (70 mg with a yield of 47%). ¹H NMR (400 MHz, DMSO-d₆) δ 10.56 (s, 1H), 8.02 (d, J=8.7 Hz, 2H), 7.92-7.83 (m, 4H), 7.79 (d, J=8.7 Hz, 2H), 7.63 (d, J=8.2 Hz, 2H), 7.34 (d, J=8.4 Hz, 1H), 3.86 (s, 2H), 3.32-3.23 (m, 2H), 2.45 (s, 3H), 1.10 (t, J=7.3 Hz, 3H). MS (ESI), m/z for C₂₄H₂₂N₂O₃S₂ ([M+H]⁺): Calcd 450.57, found 451.1.

### Example 26 N-(4-(6-methylbenzo[d]thiazol-2-yl)phenyl)heptanamide

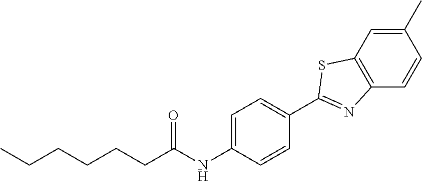 [SMILES: CCCCCCC(=O)Nc1ccc(-c2nc3ccc(C)cc3s2)cc1]

A synthesis method was as that in Example 25, and a white solid was obtained with a yield of 39%. ¹H NMR (400 MHz, DMSO-d₆) δ 10.16 (s, 1H), 8.00 (d, J=8.7 Hz, 2H), 7.92-7.86 (m, 2H), 7.78 (d, J=8.7 Hz, 2H), 7.34 (d, J=8.4 Hz, 1H), 2.45 (s, 3H), 2.35 (t, J=7.4 Hz, 2H), 1.67-1.55 (m, 2H), 1.35-1.25 (m, 6H), 0.87 (t, J=6.7 Hz, 3H). MS (ESI), m/z for C₂₁H₂₄N₂OS ([M+H]⁺): Calcd 352.50, found 353.1.

### Example 27 N-(4-(6-methylbenzo[d]thiazol-2-yl)phenyl)-2-(p-tolyl)acetamide

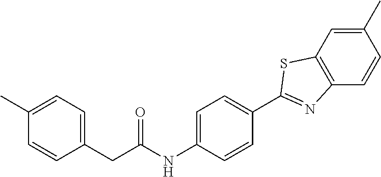

A synthesis method was as that in Example 25, and a white solid was obtained with a yield of 23%. ¹H NMR (400 MHz, DMSO-d₆) δ10.44 (s, 1H), 8.01 (d, J=8.7 Hz, 2H), 7.93-7.85 (m, 2H), 7.78 (d, J=8.7 Hz, 2H), 7.34 (d, J=8.8 Hz, 1H), 7.23 (d, J=7.9 Hz, 2H), 7.14 (d, J=7.8 Hz, 2H), 3.63 (s, 2H), 2.45 (s, 3H), 2.28 (s, 3H). MS (ESI), m/z for C₂₃H₂₀N₂OS ([M+H]⁺): Calcd 372.49, found 373.0.

### Example 28 N-(4-(6-methylbenzo[d]thiazol-2-yl)phenyl)-2-(2-nitrophenyl)acetamide

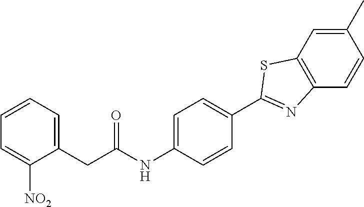 [SMILES: Cc1ccc2nc(-c3ccc(NC(=O)Cc4ccccc4[N+](=O)[O-])cc3)sc2c1]

A synthesis method was as that in Example 25, and a white solid was obtained with a yield of 35%. ¹H NMR (400 MHz, DMSO-d₆) δ 10.55 (s, 1H), 8.08 (d, J=7.9 Hz, 1H), 8.02 (d, J=8.7 Hz, 2H), 7.93-7.86 (m, 2H), 7.78-7.70 (m, 3H), 7.62-7.55 (m, 2H), 7.34 (d, J=8.8 Hz, 1H), 4.19 (s, 2H), 2.45 (s, 3H). MS (ESI), m/z for C₂₂H₁₇N₃O₃S ([M+H]⁺): Calcd 403.46, found 404.3.

### Example 29 N-(3-chloro-4-(6-methylbenzo[d]thiazol-2-yl)phenyl)-2-(4-(ethylsulfonyl)phenyl) acetamide

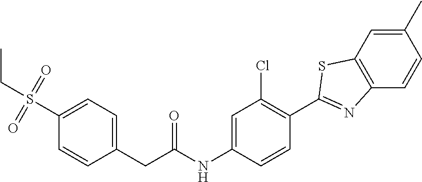 [SMILES: CCS(=O)(=O)c1ccc(CC(=O)Nc2ccc(-c3nc4ccc(C)cc4s3)c(Cl)c2)cc1]

A synthesis method was as that in Example 25, and a white solid was obtained with a yield of 42%. ¹H NMR (400 MHz, DMSO-d₆) δ 10.74 (s, 1H), 8.24 (d, J=8.7 Hz, 1H), 8.05 (d, J=1.8 Hz, 1H), 7.99-7.93 (m, 2H), 7.86 (d, J=8.2 Hz, 2H), 7.66 (dd, J=8.9, 1.9 Hz, 1H), 7.62 (d, J=8.2 Hz, 2H), 7.39 (dd, J=8.4, 0.8 Hz, 1H), 3.88 (s, 2H), 3.28 (q, J=7.6 Hz, 2H), 2.47 (s, 3H), 1.10 (t, J=7.4 Hz, 3H). MS (ESI), m/z for C₂₄H₂₁ClN₂O₃S₂ ([M+H]⁺): Calcd 485.01, found 485.0.

### Example 30 2-(4-(ethylsulfonyl)phenyl)-N-(4-(6-fluorobenzo[d]thiazol-2-yl)phenyl)acetamide

### Step 1: synthesis of N-(4-fluorophenyl)-4-nitrobenzamide

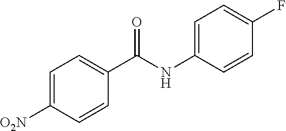 [SMILES: O=C(Nc1ccc(F)cc1)c1ccc([N+](=O)[O-])cc1]

The compound 4-fluoroaniline (2 g, 18.0 mmol) was dissolved in pyridine (10 mL), and then 4-nitrobenzoyl chloride (4.0 g, 21.6 mmol) was added to the mixture slowly to react for 1 h at 40° C. The reaction was monitored through TLC. After the reaction was finished, the reaction mixture was cooled to room temperature. Dilute hydrochloric acid (50 mL) was added to the reaction mixture and extracted three times (50 mL×3) with ethyl acetate. The organic phases were combined, washed once with saturated brine, dried over anhydrous sodium sulfate, concentrated under reduced pressure and isolated through silica gel column chromatography (PE:EA=4:1, v/v) to obtain a target compound as a white solid (3.58 g with a yield of 76%). ¹H NMR (400 MHz, DMSO-d₆) δ 10.60 (s, 1H), 8.37 (d, J=8.8 Hz, 2H), 8.18 (d, J=8.8 Hz, 2H), 7.88-7.70 (m, 2H), 7.22 (t, J=8.9 Hz, 2H).

### Step 2: synthesis of N-(4-fluorophenyl)-4-nitrobenzathioamide

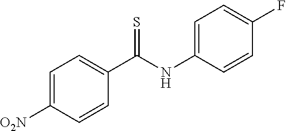 [SMILES: O=[N+]([O-])c1ccc(C(=S)Nc2ccc(F)cc2)cc1]

N-(4-fluorophenyl)-4-nitrobenzamide (3.58 g, 13.76 mmol) and Lawesson reagent (2.78 g, 6.88 mmol) were added to a solvent of 1,4-dioxane (20 mL), and the mixture was heated to 110° C. and stirred for 3 h. After the reaction was finished, the reaction system was cooled to room temperature and concentrated under reduced pressure. Then, water was added to the reaction system, and a precipitated solid was suction filtered. A filter cake was washed with water, dried and recrystallized with methanol to obtain a target compound as an orange solid (2.74 g with a yield of 72%). ¹H NMR (400 MHz, DMSO-d₆) δ 12.16 (s, 1H), 8.29 (d, J=8.7 Hz, 2H), 8.01 (d, J=8.6 Hz, 2H), 7.94-7.80 (m, 2H), 7.29 (t, J=8.8 Hz, 2H).

### Step 3: synthesis of 6-fluoro-2-(4-nitrophenyl)benzo[d]thiazol

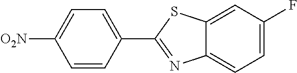 [SMILES: O=[N+]([O-])c1ccc(-c2nc3ccc(F)cc3s2)cc1]

N-(4-fluorophenyl)-4-nitrobenzathioamide (2.0 g, 7.24 mmol) was dissolved in an aqueous solution of sodium hydroxide (2.9 g, 72.4 mmol) containing ethanol (3 mL) and water (30 mL), and then an aqueous solution (20 mL) of potassium ferricyanide (9.54 g, 28.98 mmol) was added dropwise to the solution. The mixture was stirred for 30 min at 90° C. After the reaction was finished, the reaction system was cooled to room temperature, and a precipitate was precipitated. The precipitated solid was suction filtered. A filter cake was washed with water, dried and recrystallized (EA:PE=1:2, v/v) to obtain a target compound as a yellow solid (1.6 g with a yield of 81%). ¹H NMR (400 MHz, DMSO-d₆) δ 8.38 (d, J=8.8 Hz, 2H), 8.32 (d, J=8.8 Hz, 2H), 8.20-8.11 (m, 2H), 7.47 (td, J=9.1, 2.5 Hz, 1H).

### Step 4: Synthesis of 4-(6-fluorobenzo[d]thiazol-2-yl) aniline

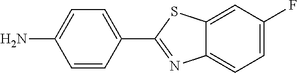 [SMILES: Nc1ccc(-c2nc3ccc(F)cc3s2)cc1]

The compound 6-fluoro-2-(4-nitrophenyl)benzo[d]thiazol (0.8 g, 2.92 mmol) and 10% palladium on carbon (about 55% water content) (160 mg) were added to a solvent of MeOH (10 mL) and stirred overnight at room temperature in a hydrogen environment. After the reaction was finished, the mixture was suction filtered with Celite as a filter aid, and a filtrate was concentrated to obtain a crude product. The crude product was isolated through the silica gel column chromatography (PE:EA=4:1, v/v) to obtain a target compound as a white solid (0.5 g with a yield of 70%). ¹H NMR (400 MHz, DMSO-d₆) δ 8.00-7.85 (m, 2H), 7.73 (d, J=8.5 Hz, 2H), 7.30 (td, J=9.0, 2.5 Hz, 1H), 6.67 (d, J=8.5 Hz, 2H), 5.88 (s, 2H).

### Step 5: synthesis of 2-(4-(ethylsulfonyl)phenyl)-N-(4-(6-fluorobenzo[d]thiazol-2-yl)phenyl) acetamide

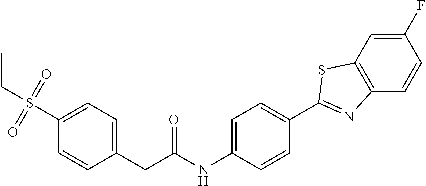 [SMILES: CCS(=O)(=O)c1ccc(CC(=O)Nc2ccc(-c3nc4ccc(F)cc4s3)cc2)cc1]

A synthesis method was as that in Example 25, and a white solid was obtained with a yield of 56%. ¹H NMR (400 MHz, DMSO-d₆) δ 10.59 (s, 1H), 8.07-7.98 (m, 4H), 7.85 (d, J=8.2 Hz, 2H), 7.80 (d, J=8.7 Hz, 2H), 7.63 (d, J=8.2 Hz, 2H), 7.39 (td, J=9.1, 2.6 Hz, 1H), 3.86 (s, 2H), 3.27 (q, J=7.2 Hz, 2H), 1.10 (t, J=7.3 Hz, 3H). MS (ESI), m/z for C₂₃H₁₉FN₂O₃S₂ ([M+H]⁺): Calcd 454.53, found 455.0.

### Example 31 2-(4-(ethylsulfonyl)phenyl)-N-(4-(6-methoxybenzo[d]thiazol-2-yl)phenyl) acetamide

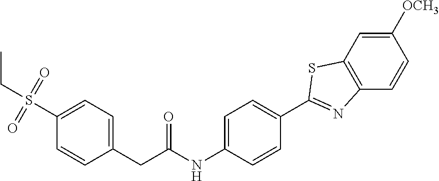 [SMILES: CCS(=O)(=O)c1ccc(CC(=O)Nc2ccc(-c3nc4ccc(OC)cc4s3)cc2)cc1]

A synthesis method was as that in Example 30, and a white solid was obtained with a yield of 56%. ¹H NMR (400 MHz, DMSO-d₆) δ 10.55 (s, 1H), 7.99 (d, J=8.7 Hz, 2H), 7.89 (d, J=8.9 Hz, 1H), 7.85 (d, J=8.2 Hz, 2H), 7.77 (d, J=8.7 Hz, 2H), 7.69 (d, J=2.5 Hz, 1H), 7.62 (d, J=8.3 Hz, 2H), 7.11 (dd, J=8.9, 2.6 Hz, 1H), 3.85 (s, 2H), 3.84 (s, 3H), 3.27 (q, J=7.6 Hz, 2H), 1.10 (t, J=7.3 Hz, 3H). MS (ESI), m/z for C₂₄H₂₂N₂O₄S₂ ([M+Na]+): Calcd 466.57, found 489.7.

### Example 32 2-(4-(Ethylsulfonyl)phenyl)-N-(4-(6-methyl-1-(4-methylphenylethyl)-1H-benzo[d]imidazol-2-yl)phenyl)acetamide

### Step 1: synthesis of 5-methyl-N-(4-methylphenylethyl)-2-nitroaniline

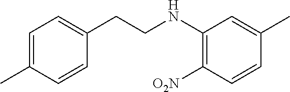

The compound 3-fluoro-4-nitrotoluene (2.0 g, 12.9 mmol) was dissolved in DMSO (5 mL), and p-methylphenethylamine (5.23 g, 38.7 mmol) and DIPEA (2.51 g, 19.4 mmol) were added to the mixture. The reaction mixture was stirred overnight at 95° C. The reaction was monitored through TLC. After the reaction was finished, water was added to the reaction mixture and extracted with ethyl acetate (50 mL×3). Combined organic layers were dried over anhydrous Na₂S₀₄ and concentrated under reduced pressure. A crude product was isolated through silica gel column chromatography (PE:EA=50:1, v/v) to obtain a target compound as a yellow solid (2.8 g with a yield of 80%). ¹H NMR (400 MHz, DMSO-d₆) δ 8.09 (s, 1H), 7.94 (d, J=8.4 Hz, 1H), 7.19 (d, J=6.6 Hz, 1H), 7.12 (d, J=5.7 Hz, 1H), 6.88 (s, 1H), 6.51 (d, J=7.9 Hz, 1H), 3.62-3.50 (m, 2H), 2.90 (t, J=6.8 Hz, 2H), 2.31 (s, 3H), 2.27 (s, 3H).

### Step 2: synthesis of 5-methyl-N-(4-methylphenylethyl)benzene-1,2-diamine

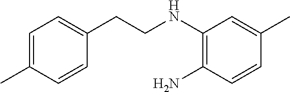

Iron powder (3.46 g, 61.8 mmol), ammonium chloride (551.1 mg, 10.3 mmol) and acetic acid (1.24 g, 20.6 mmol) were added to water (20 mL), heated to 50° C. and stirred for 10 min. The compound 5-methyl-N-(4-methylphenylethyl)-2-nitroaniline (2.8 g, 10.3 mmol) was dissolved in DMF (15 mL) and quickly added to the above mixed solution. Stirring was continued for 1 h, and the reaction was monitored through the TLC. After the reaction was finished, the mixture was cooled to room temperature and suction filtered. An aqueous layer had a pH adjusted to 8 to 9 with a solution of sodium carbonate and extracted with ethyl acetate (50 mL×3). Organic layers were combined, washed with a saturated solution of sodium chloride, dried over anhydrous Na₂SO₄ and concentrated under reduced pressure to obtain a crude product. The crude product was isolated through the silica gel column chromatography (PE:EA=10:1, v/v) to obtain a target compound as a yellow oily liquid (2.1 g with a yield of 81%). ¹H NMR (400 MHz, DMSO-d₆) δ 7.17 (d, J=8.0 Hz, 2H), 7.11 (d, J=7.9 Hz, 2H), 6.43 (d, J=7.6 Hz, 1H), 6.28 (s, 1H), 6.22 (d, J=7.6 Hz, 1H), 4.36 (t, J=5.5 Hz, 1H), 4.21 (s, 2H), 3.23-3.15 (m, 2H), 2.83 (t, J=7.4 Hz, 2H), 2.27 (s, 3H), 2.12 (s, 3H).

### Step 3: synthesis of 6-methyl-1-(4-methylphenylethyl)-2-(4-nitrophenyl)-1H-benzo[d]imidazole

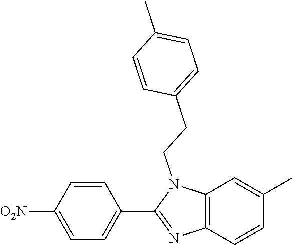 [SMILES: Cc1ccc(CCn2c(-c3ccc([N+](=O)[O-])cc3)nc3ccc(C)cc32)cc1]

5-methyl-N¹-(4-methylphenylethyl)benzene-1,2-diamine (1.3 g, 5.4 mmol), p-nitrobenzaldehyde (816.0 mg, 5.4 mmol) and oxone (1.84 g, 3.0 mmol) were added to DMF (20 mL) and stirred for 1 h at room temperature. The reaction was monitored through the TLC. After the reaction was finished, water was added to the mixture and extracted with ethyl acetate (50 mL×3). Organic layers were combined, washed with a saturated solution of sodium chloride, dried over anhydrous Na₂SO₄ and concentrated under reduced pressure to obtain a crude product. The crude product was purified through the silica gel column chromatography (PE:EA=2:1, v/v) to obtain a target compound as a yellow solid (1.78 g with a yield of 86%). ¹H NMR (400 MHz, CDCl₃) δ 8.21 (d, J=8.8 Hz, 2H), 7.72 (d, J=8.2 Hz, 1H), 7.48 (d, J=8.8 Hz, 2H), 7.27 (s, 1H), 7.19 (d, J=8.3 Hz, 1H), 6.94 (d, J=7.8 Hz, 2H), 6.64 (d, J=7.9 Hz, 2H), 4.45 (t, J=6.8 Hz, 2H), 3.03 (t, J=6.8 Hz, 2H), 2.57 (s, 3H), 2.29 (s, 3H).

### Step 4: synthesis of 4-(6-methyl-1-(4-methylphenylethyl)-1H-benzo[d]imidazol-2-yl)aniline

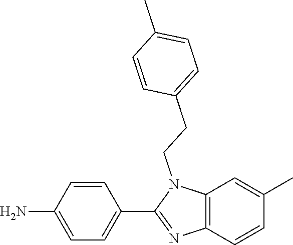 [SMILES: Cc1ccc(CCn2c(-c3ccc(N)cc3)nc3ccc(C)cc32)cc1]

The intermediate 6-methyl-1-(4-methylphenylethyl)-2-(4-nitrophenyl)-1H-benzo[d]imidazole was used as a raw material. For a synthesis method, reference was made to step 2. A yellow solid was obtained with a yield of 85%. ¹H NMR (400 MHz, DMSO) δ 7.46 (d, J=8.1 Hz, 1H), 7.36 (s, 1H), 7.28 (d, J=8.5 Hz, 2H), 7.04 (d, J=7.8 Hz, 2H), 7.00 (dd, J=8.3, 1.0 Hz, 1H), 6.97 (d, J=8.0 Hz, 2H), 6.65 (d, J=8.5 Hz, 2H), 5.51 (s, 2H), 4.34 (t, J=8.0 Hz, 2H), 2.97 (t, J=7.6 Hz, 2H), 2.45 (s, 3H), 2.25 (s, 3H).

### Step 5: synthesis of 2-(4-(ethylsulfonyl)phenyl)-N-(4-(6-methyl-1-(4-methylphenylethyl)-1H-benzo[d]imidazol-2-yl)phenyl)acetamide

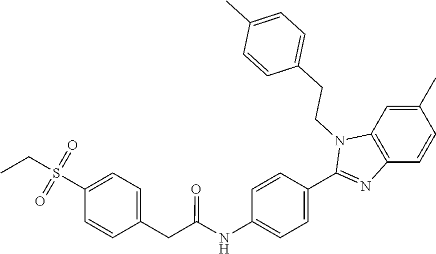 [SMILES: CCS(=O)(=O)c1ccc(CC(=O)Nc2ccc(-c3nc4ccc(C)cc4n3CCc3ccc(C)cc3)cc2)cc1]

The compounds 4-(6-methyl-1-(4-methylphenylethyl)-1H-benzo[d]imidazol-2-yl)aniline (150 mg, 0.44 mmol), diisopropylethylamine (170.3 mg, 1.32 mmol) and HATU (250.8 mg, 0.66 mmol) were dissolved in DCM (10 mL). The reaction mixture was stirred for 15 min, then the compound 2-(4-(ethylsulfonyl)phenyl)acetic acid (121.0 mg, 0.53 mmol) was added to the reaction mixture, and the obtained mixture was stirred overnight at room temperature. After the reaction was finished, water was added to dilute the mixture and extracted with DCM (50 mL×3). Organic layers were combined, dried over anhydrous Na₂SO₄ and concentrated under reduced pressure. A crude product was isolated through the silica gel column chromatography (DCM:CH₃OH=70:1, v/v) to obtain a target compound as a white solid (70 mg with a yield of 29%). ¹H NMR (400 MHz, DMSO-d₆) δ 10.47 (s, 1H), 7.87 (d, J=8.3 Hz, 2H), 7.71 (d, J=8.6 Hz, 2H), 7.64 (d, J=8.3 Hz, 2H), 7.51 (d, J=8.2 Hz, 1H), 7.48 (d, J=8.7 Hz, 2H), 7.43 (s, 1H), 7.05 (dd, J=8.2, 0.8 Hz, 1H), 6.98 (d, J=7.7 Hz, 2H), 6.86 (d, J=7.9 Hz, 2H), 4.39 (t, J=7.3 Hz, 2H), 3.86 (s, 2H), 3.28 (q, J=7.5 Hz, 2H), 2.95 (t, J=7.3 Hz, 2H), 2.47 (s, 3H), 2.23 (s, 3H), 1.11 (t, J=7.4 Hz, 3H). MS (ESI), m/z for C₃₃H₃₃N₃O₃S ([M−H]⁻): Calcd 551.71, found 550.4.

### Example 33 2-(4-(ethylsulfonyl)phenyl)-N-(4-(6-methyl-1-propyl-1H-benzo[d]imidazol-2-yl)phenyl)acetamide

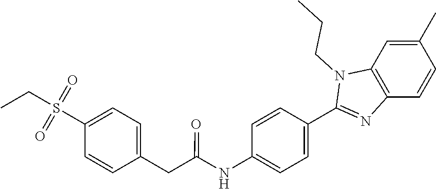 [SMILES: CCCn1c(-c2ccc(NC(=O)Cc3ccc(S(=O)(=O)CC)cc3)cc2)nc2ccc(C)cc21]

A synthesis method was as that in Example 32, and a white solid was obtained with a yield of 45%. ¹H NMR (500 MHz, DMSO-d₆) δ 10.51 (s, 1H), 7.86 (d, J=8.2 Hz, 2H), 7.79 (d, J=8.6 Hz, 2H), 7.70 (d, J=8.8 Hz, 2H), 7.64 (d, J=8.2 Hz, 2H), 7.52 (d, J=8.2 Hz, 1H), 7.42 (s, 1H), 7.05 (d, J=8.1 Hz, 1H), 4.21 (t, J=7.4 Hz, 2H), 3.86 (s, 2H), 3.28 (q, J=7.5 Hz, 2H), 2.46 (s, 3H), 1.71-1.64 (m, 2H), 1.10 (t, J=7.4 Hz, 3H), 0.73 (t, J=7.3 Hz, 3H). MS (ESI), m/z for C₂₇H₂₉N₃O₃S ([M+H]⁺): Calcd 475.61, found 476.3.

### Example 34 N-(4-(1-butyl-6-methyl-1H-benzo[d]imidazol-2-yl)phenyl)-2-(4-(ethylsulfonyl) phenyl)acetamide

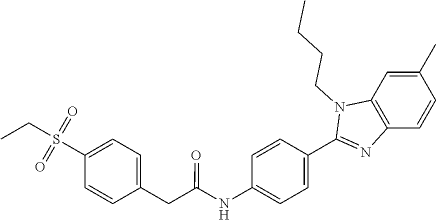 [SMILES: CCCCn1c(-c2ccc(NC(=O)Cc3ccc(S(=O)(=O)CC)cc3)cc2)nc2ccc(C)cc21]

A synthesis method was as that in Example 32, and a white solid was obtained with a yield of 26%. ¹H NMR (400 MHz, DMSO-d₆) δ 10.53 (s, 1H), 7.87 (d, J=8.2 Hz, 2H), 7.80 (d, J=8.6 Hz, 2H), 7.71 (d, J=8.6 Hz, 2H), 7.64 (d, J=8.2 Hz, 2H), 7.53 (d, J=8.2 Hz, 1H), 7.45 (s, 1H), 7.08 (d, J=8.1 Hz, 1H), 4.26 (t, J=7.3 Hz, 2H), 3.86 (s, 2H), 3.33-3.23 (m, 2H), 2.47 (s, 3H), 1.71-1.59 (m, 2H), 1.19-1.07 (m, 5H), 0.76 (t, J=7.3 Hz, 3H). MS (ESI), m/z for C₂₈H₃₁N₃O₃S ([M+H]⁺): Calcd 489.63, found 490.5.

### Example 35 2-(4-(ethylsulfonyl)phenyl)-N-(4-(6-methyl-1-pentyl-1H-benzo[d]imidazol-2-yl) phenyl)acetamide

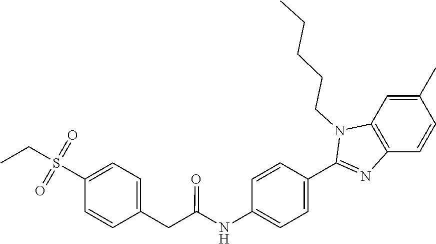 [SMILES: CCCCCn1c(-c2ccc(NC(=O)Cc3ccc(S(=O)(=O)CC)cc3)cc2)nc2ccc(C)cc21]

A synthesis method was as that in Example 32, and a white solid was obtained with a yield of 31%. ¹H NMR (400 MHz, DMSO-d₆) δ 10.50 (s, 1H), 7.86 (d, J=8.2 Hz, 2H), 7.79 (d, J=8.6 Hz, 2H), 7.70 (d, J=8.6 Hz, 2H), 7.64 (d, J=8.2 Hz, 2H), 7.52 (d, J=8.2 Hz, 1H), 7.41 (s, 1H), 7.06 (d, J=8.1 Hz, 1H), 4.24 (t, J=7.3 Hz, 2H), 3.86 (s, 2H), 3.28 (q, J=7.2 Hz, 2H), 2.46 (s, 3H), 1.70-1.61 (m, 2H), 1.20-1.06 (m, 7H), 0.75 (t, J=7.0 Hz, 3H). MS (ESI), m/z for C₂₉H₃₃N₃O₃S ([M+H]⁺): Calcd 503.66, found 504.7.

### Example 36 2-(4-(ethylsulfonyl)phenyl)-N-(4-(1-isopropyl-6-methyl-1H-benzo[d]imidazol-2-yl)phenyl)acetamide

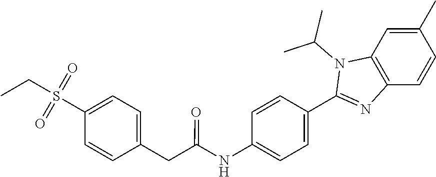 [SMILES: CCS(=O)(=O)c1ccc(CC(=O)Nc2ccc(-c3nc4ccc(C)cc4n3C(C)C)cc2)cc1]

A synthesis method was as that in Example 32, and a white solid was obtained with a yield of 28%. ¹H NMR (400 MHz, DMSO) δ 10.52 (s, 1H), 7.86 (d, J=8.2 Hz, 2H), 7.78 (d, J=8.5 Hz, 2H), 7.64 (d, J=8.2 Hz, 2H), 7.61-7.56 (m, 3H), 7.51 (d, J=8.2 Hz, 1H), 7.03 (d, J=8.1 Hz, 1H), 4.75-4.63 (m, 1H), 3.86 (s, 2H), 3.28 (q, J=7.3 Hz, 2H), 2.46 (s, 3H), 1.57 (d, J=6.9 Hz, 6H), 1.11 (t, J=7.3 Hz, 3H). MS (ESI), m/z for C₂₇H₂₉N₃O₃S ([M+H]⁺): Calcd 475.61, found 476.3.

### Example 37 2-(4-(Ethylsulfonyl)phenyl)-N-(4-(1-isobutyl-6-methyl-1H-benzo[d]imidazol-2-yl)phenyl)acetamide

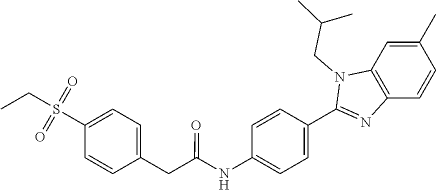 [SMILES: CCS(=O)(=O)c1ccc(CC(=O)Nc2ccc(-c3nc4ccc(C)cc4n3CC(C)C)cc2)cc1]

A synthesis method was as that in Example 32, and a white solid was obtained with a yield of 27%. ¹H NMR (400 MHz, DMSO-d₆) δ 10.54 (s, 1H), 7.87 (d, J=8.2 Hz, 2H), 7.80 (d, J=8.6 Hz, 2H), 7.73 (d, J=8.6 Hz, 2H), 7.64 (d, J=8.2 Hz, 2H), 7.55 (d, J=8.2 Hz, 1H), 7.50 (s, 1H), 7.10 (d, J=8.2 Hz, 1H), 4.16 (d, J=7.5 Hz, 2H), 3.87 (s, 2H), 3.28 (q, J=7.2 Hz, 2H), 2.47 (s, 3H), 2.01-1.87 (m, 1H), 1.11 (t, J=7.3 Hz, 3H), 0.64 (d, J=6.6 Hz, 6H). MS (ESI), m/z for C₂₈H₃₁N₃O₃S ([M+H]⁺): Calcd 489.63, found 490.5.

### Example 38 2-(4-(ethylsulfonyl)phenyl)-N-(4-(1-isopentyl-6-methyl-1H-benzo[d]imidazol-2-yl)phenyl)acetamide

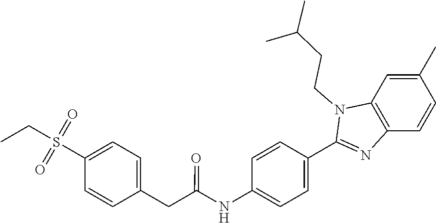 [SMILES: CCS(=O)(=O)c1ccc(CC(=O)Nc2ccc(-c3nc4ccc(C)cc4n3CCC(C)C)cc2)cc1]

A synthesis method was as that in Example 32, and a white solid was obtained with a yield of 41%. ¹H NMR (400 MHz, DMSO-d₆) δ 10.50 (s, 1H), 7.87 (d, J=6.9 Hz, 2H), 7.80 (d, J=7.4 Hz, 2H), 7.70 (d, J=7.3 Hz, 2H), 7.64 (d, J=7.0 Hz, 2H), 7.52 (d, J=7.4 Hz, 1H), 7.39 (s, 1H), 7.05 (d, J=7.2 Hz, 1H), 4.33-4.16 (m, 2H), 3.87 (s, 2H), 3.32-3.22 (m, 2H), 2.47 (s, 3H), 1.63-1.52 (m, 2H), 1.51-1.41 (m, 1H), 1.11 (t, J=7.4 Hz, 3H), 0.79 (d, J=5.0 Hz, 6H). MS (ESI), m/z for C₂₉H₃₃N₃O₃S ([M+H]⁺): Calcd 503.66, found 504.7.

### Example 39 N-(4-(1-cyclobutyl-6-methyl-1H-benzo[d]imidazol-2-yl)phenyl)-2-(4-(ethylsulfonyl)phenyl)acetamide

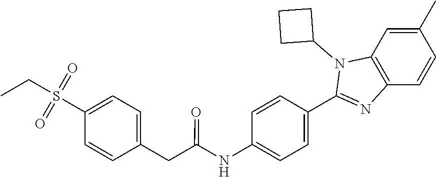 [SMILES: CCS(=O)(=O)c1ccc(CC(=O)Nc2ccc(-c3nc4ccc(C)cc4n3C3CCC3)cc2)cc1]

A synthesis method was as that in Example 32, and a white solid was obtained with a yield of 30%. ¹H NMR (400 MHz, DMSO-d₆) δ 10.52 (s, 1H), 7.87 (d, J=8.2 Hz, 2H), 7.79 (d, J=8.5 Hz, 2H), 7.67-7.58 (m, 5H), 7.54 (d, J=8.2 Hz, 1H), 7.09 (d, J=8.2 Hz, 1H), 5.13-5.01 (m, 1H), 3.87 (s, 2H), 3.28 (q, J=7.2 Hz, 2H), 2.76-2.61 (m, 2H), 2.49 (s, 3H), 2.43-2.32 (m, 2H), 1.95-1.70 (m, 2H), 1.11 (t, J=7.3 Hz, 3H). MS (ESI), m/z for C₂₈H₂₉N₃O₃S ([M+H]⁺): Calcd 487.62, found 488.5.

### Example 40 N-(4-(1-(cyclobutylmethyl)-6-methyl-1H-benzo[d]imidazol-2-yl)phenyl)-2-(4-(ethylsulfonyl)phenyl)acetamide

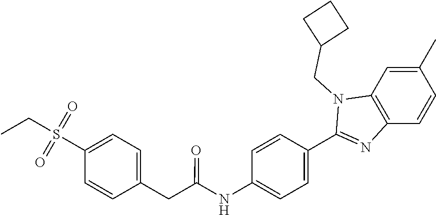 [SMILES: CCS(=O)(=O)c1ccc(CC(=O)Nc2ccc(-c3nc4ccc(C)cc4n3CC3CCC3)cc2)cc1]

A synthesis method was as that in Example 32, and a white solid was obtained with a yield of 22%. ¹H NMR (400 MHz, DMSO-d₆) δ 10.50 (s, 1H), 7.87 (d, J=8.1 Hz, 2H), 7.79 (d, J=8.5 Hz, 2H), 7.70 (d, J=8.5 Hz, 2H), 7.64 (d, J=8.1 Hz, 2H), 7.50 (d, J=8.2 Hz, 1H), 7.44 (s, 1H), 7.04 (d, J=8.2 Hz, 1H), 4.35 (d, J=7.2 Hz, 2H), 3.86 (s, 2H), 3.32-3.24 (m, 2H), 2.60-2.51 (m, 1H), 2.46 (s, 3H), 1.78-1.57 (m, 4H), 1.54-1.44 (m, 2H), 1.11 (t, J=7.3 Hz, 3H). MS (ESI), m/z for C₂₉H₃₁N₃O₃S ([M+H]⁺): Calcd 501.65, found 502.5.

### Example 41 N-(4-(1-(cyclohexylmethyl)-6-methyl-1H-benzo[d]imidazol-2-yl)phenyl)-2-(4-(ethylsulfonyl)phenyl)acetamide

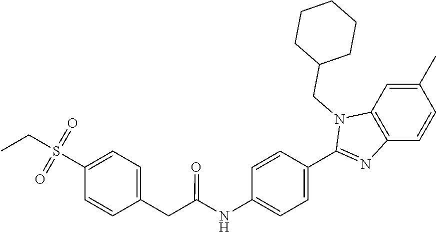 [SMILES: CCS(=O)(=O)c1ccc(CC(=O)Nc2ccc(-c3nc4ccc(C)cc4n3CC3CCCCC3)cc2)cc1]

A synthesis method was as that in Example 32, and a white solid was obtained with a yield of 40%. ¹H NMR (400 MHz, DMSO-d₆) δ 10.50 (s, 1H), 7.87 (d, J=8.2 Hz, 2H), 7.78 (d, J=8.6 Hz, 2H), 7.70 (d, J=8.5 Hz, 2H), 7.64 (d, J=8.1 Hz, 2H), 7.51 (d, J=8.1 Hz, 1H), 7.42 (s, 1H), 7.04 (d, J=8.2 Hz, 1H), 4.17 (d, J=7.2 Hz, 2H), 3.86 (s, 2H), 3.28 (q, J=7.2 Hz, 2H), 2.46 (s, 3H), 1.69-1.56 (m, 1H), 1.53-1.42 (m, 4H), 1.34-1.19 (m, 6H), 1.11 (t, J=7.6 Hz, 3H). MS (ESI), m/z for C₃₁H₃₅N₃O₃S ([M+H]⁺): Calcd 529.70, found 530.7.

### Example 42 N-(4-(1-benzyl-6-methyl-1H-benzo[d]imidazol-2-yl)phenyl)-2-(4-(ethylsulfonyl) phenyl)acetamide

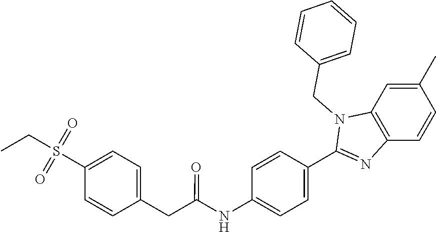 [SMILES: CCS(=O)(=O)c1ccc(CC(=O)Nc2ccc(-c3nc4ccc(C)cc4n3Cc3ccccc3)cc2)cc1]

A synthesis method was as that in Example 32, and a white solid was obtained with a yield of 20%. ¹H NMR (400 MHz, DMSO-d₆) δ 10.48 (s, 1H), 7.85 (d, J=8.2 Hz, 2H), 7.72 (d, J=8.7 Hz, 2H), 7.66 (d, J=8.7 Hz, 2H), 7.64-7.55 (m, 3H), 7.32-7.20 (m, 4H), 7.08 (d, J=8.1 Hz, 1H), 6.99 (d, J=7.2 Hz, 2H), 5.55 (s, 2H), 3.84 (s, 2H), 3.32-3.22 (m, 2H), 2.39 (s, 3H), 1.10 (t, J=7.3 Hz, 3H). MS (ESI), m/z for C₃₁H₂₉N₃O₃S ([M+H]⁺): Calcd 523.65, found 524.3.

### Example 43 2-(4-(ethylsulfonyl)phenyl)-N-(4-(6-methyl-1-(4-methylbenzyl)-1H-benzo[d]imidazol-2-yl)phenyl)acetamide

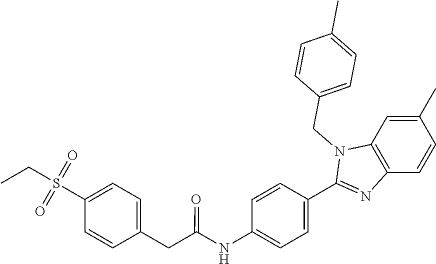 [SMILES: CCS(=O)(=O)c1ccc(CC(=O)Nc2ccc(-c3nc4ccc(C)cc4n3Cc3ccc(C)cc3)cc2)cc1]

A synthesis method was as that in Example 32, and a white solid was obtained with a yield of 25%. ¹H NMR (400 MHz, DMSO-d₆) δ 10.49 (s, 1H), 7.85 (d, J=8.2 Hz, 2H), 7.73 (d, J=8.7 Hz, 2H), 7.66 (d, J=8.7 Hz, 2H), 7.62 (d, J=8.2 Hz, 2H), 7.57 (d, J=8.2 Hz, 1H), 7.25 (s, 1H), 7.12-7.03 (m, 3H), 6.88 (d, J=7.9 Hz, 2H), 5.49 (s, 2H), 3.84 (s, 2H), 3.27 (q, J=7.2 Hz, 2H), 2.39 (s, 3H), 2.22 (s, 3H), 1.10 (t, J=7.3 Hz, 3H). MS (ESI), m/z for C₃₂H₃₁N₃O₃S ([M+H]⁺): Calcd 537.68, found 538.6.

### Example 44 2-(4-(ethylsulfonyl)phenyl)-N-(4-(6-methyl-1-phenylethyl-1H-benzo[d]imidazol-2-yl)phenyl)acetamide

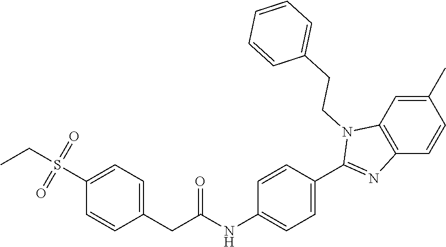 [SMILES: CCS(=O)(=O)c1ccc(CC(=O)Nc2ccc(-c3nc4ccc(C)cc4n3CCc3ccccc3)cc2)cc1]

A synthesis method was as that in Example 32, and a white solid was obtained with a yield of 45%. ¹H NMR (400 MHz, DMSO-d₆) δ 10.48 (s, 1H), 7.87 (d, J=8.2 Hz, 2H), 7.72 (d, J=8.5 Hz, 2H), 7.64 (d, J=8.2 Hz, 2H), 7.55-7.47 (m, 3H), 7.45 (s, 1H), 7.22-7.13 (m, 3H), 7.06 (d, J=8.2 Hz, 1H), 7.02-6.95 (m, 2H), 4.43 (t, J=7.3 Hz, 2H), 3.86 (s, 2H), 3.28 (q, J=7.6 Hz, 2H), 3.01 (t, J=7.3 Hz, 2H), 2.47 (s, 3H), 1.11 (t, J=7.3 Hz, 3H). MS (ESI), m/z for C₃₂H₃₁N₃O₃S ([M+H]⁺): Calcd 537.68, found 538.6.

### Example 45 2-(4-(ethylsulfonyl)phenyl)-N-(4-(1-(4-fluorobenzyl)-6-methyl-1H-benzo[d]imidazol-2-yl)phenyl)acetamide

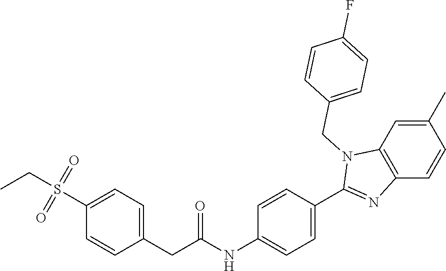 [SMILES: CCS(=O)(=O)c1ccc(CC(=O)Nc2ccc(-c3nc4ccc(C)cc4n3Cc3ccc(F)cc3)cc2)cc1]

A synthesis method was as that in Example 32, and a white solid was obtained with a yield of 26%. ¹H NMR (400 MHz, DMSO-d₆) δ 10.48 (s, 1H), 7.85 (d, J=8.3 Hz, 2H), 7.73 (d, J=8.7 Hz, 2H), 7.65 (d, J=8.7 Hz, 2H), 7.62 (d, J=8.2 Hz, 2H), 7.57 (d, J=8.2 Hz, 1H), 7.28 (s, 1H), 7.11 (t, J=8.8 Hz, 2H), 7.07 (d, J=8.4 Hz, 1H), 7.04-6.98 (m, 2H), 5.53 (s, 2H), 3.84 (s, 2H), 3.27 (q, J=7.2 Hz, 2H), 2.40 (s, 3H), 1.10 (t, J=7.3 Hz, 3H). MS (ESI), m/z for C₃₁H₂₈FN₃O₃S ([M+H]⁺): Calcd 541.64, found 542.3.

### Example 46 2-(4-(ethylsulfonyl)phenyl)-N-(4-(1-(3-fluorobenzyl)-6-methyl-1H-benzo[d]imidazol-2-yl)phenyl)acetamide

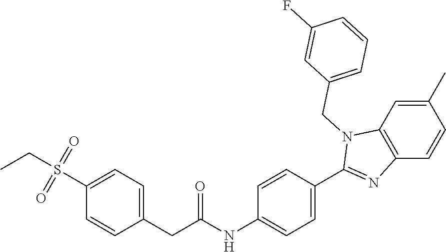 [SMILES: CCS(=O)(=O)c1ccc(CC(=O)Nc2ccc(-c3nc4ccc(C)cc4n3Cc3cccc(F)c3)cc2)cc1]

A synthesis method was as that in Example 32, and a white solid was obtained with a yield of 29%. ¹H NMR (400 MHz, DMSO-d₆) δ 10.48 (s, 1H), 7.85 (d, J=8.3 Hz, 2H), 7.73 (d, J=8.7 Hz, 2H), 7.69-7.56 (m, 5H), 7.36-7.29 (m, 1H), 7.28 (s, 1H), 7.08 (d, J=8.3 Hz, 2H), 6.82 (d, J=9.9 Hz, 1H), 6.76 (d, J=7.8 Hz, 1H), 5.56 (s, 2H), 3.84 (s, 2H), 3.31-3.23 (m, 2H), 2.40 (s, 3H), 1.10 (t, J=7.4 Hz, 3H). MS (ESI), m/z for C₃₁H₂₈FN₃O₃S ([M+H]⁺): Calcd 541.64, found 542.3.

### Example 47 2-(4-(ethylsulfonyl)phenyl)-N-(4-(1-(2-fluorobenzyl)-6-methyl-1H-benzo[d]imidazol-2-yl)phenyl)acetamide

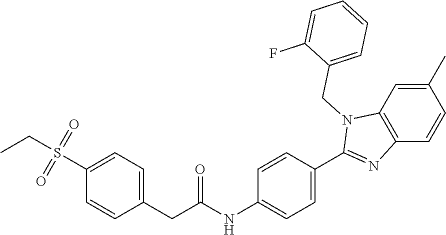 [SMILES: CCS(=O)(=O)c1ccc(CC(=O)Nc2ccc(-c3nc4ccc(C)cc4n3Cc3ccccc3F)cc2)cc1]

A synthesis method was as that in Example 32, and a white solid was obtained with a yield of 25%. ¹H NMR (400 MHz, DMSO-d₆) δ 10.48 (s, 1H), 7.85 (d, J=8.2 Hz, 2H), 7.72 (d, J=8.6 Hz, 2H), 7.67-7.55 (m, 5H), 7.34-7.25 (m, 2H), 7.20 (t, J=8.7 Hz, 1H), 7.11-7.02 (m, 2H), 6.69 (t, J=7.6 Hz, 1H), 5.57 (s, 2H), 3.84 (s, 2H), 3.27 (q, J=7.2 Hz, 2H), 2.39 (s, 3H), 1.10 (t, J=7.3 Hz, 3H). MS (ESI), m/z for C₃₁H₂₈FN₃O₃S ([M+H]⁺): Calcd 541.64, found 542.3.

### Example 48 N-(4-(1-(2,6-difluorobenzyl)-6-methyl-1H-benzo[d]imidazol-2-yl)phenyl)-2-(4-(ethylsulfonyl)phenyl)acetamide

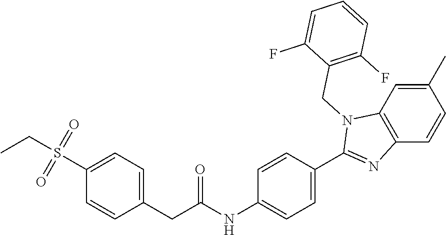 [SMILES: CCS(=O)(=O)c1ccc(CC(=O)Nc2ccc(-c3nc4ccc(C)cc4n3Cc3c(F)cccc3F)cc2)cc1]

A synthesis method was as that in Example 32, and a white solid was obtained with a yield of 35%. ¹H NMR (400 MHz, DMSO-d₆) δ 10.49 (s, 1H), 7.86 (d, J=8.2 Hz, 2H), 7.76 (d, J=8.6 Hz, 2H), 7.69 (d, J=8.6 Hz, 2H), 7.64 (d, J=8.1 Hz, 2H), 7.52 (d, J=8.1 Hz, 1H), 7.39-7.28 (m, 1H), 7.18 (s, 1H), 7.07-6.94 (m, 3H), 5.60 (s, 2H), 3.86 (s, 2H), 3.28 (q, J=7.6 Hz, 2H), 2.38 (s, 3H), 1.10 (t, J=7.3 Hz, 3H). MS (ESI), m/z for C₃₁H₂₇F₂N₃O₃S ([M+H]⁺): Calcd 559.63, found 560.3.

### Example 49 N-(4-(1-(4-cyanobenzyl)-6-methyl-1H-benzo[d]imidazol-2-yl)phenyl)-2-(4-(ethylsulfonyl)phenyl)acetamide

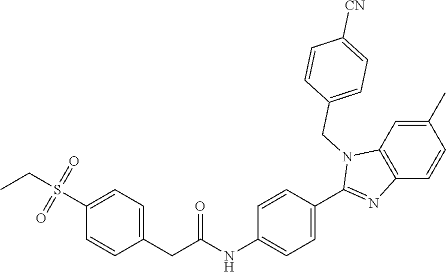 [SMILES: CCS(=O)(=O)c1ccc(CC(=O)Nc2ccc(-c3nc4ccc(C)cc4n3Cc3ccc(C#N)cc3)cc2)cc1]

A synthesis method was as that in Example 32, and a white solid was obtained with a yield of 31%. H NMR (400 MHz, DMSO-d₆) δ 10.47 (s, 1H), 7.85 (d, J=8.3 Hz, 2H), 7.76 (d, J=8.3 Hz, 2H), 7.71 (d, J=8.7 Hz, 2H), 7.66-7.55 (m, 5H), 7.26 (s, 1H), 7.14 (d, J=8.3 Hz, 2H), 7.08 (d, J=8.3 Hz, 1H), 5.65 (s, 2H), 3.83 (s, 2H), 3.31-3.23 (m, 2H), 2.39 (s, 3H), 1.10 (t, J=7.3 Hz, 3H). MS (ESI), m/z for C₃₂H₂₈N₄O₃S ([M+H]⁺): Calcd 548.66, found 549.2.

### Example 50 2-(4-(ethylsulfonyl)phenyl)-N-(4-(6-methyl-1-(4-(methylsulfonyl)benzyl)-1H-benzo[d]imidazol-2-yl)phenyl)acetamide

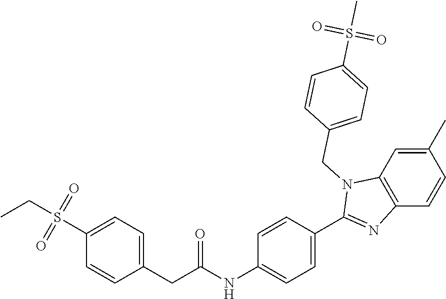 [SMILES: CCS(=O)(=O)c1ccc(CC(=O)Nc2ccc(-c3nc4ccc(C)cc4n3Cc3ccc(S(C)(=O)=O)cc3)cc2)cc1]

A synthesis method was as that in Example 32, and a white solid was obtained with a yield of 49%. ¹H NMR (400 MHz, DMSO-d₆) δ 10.49 (s, 1H), 7.90-7.81 (m, 4H), 7.72 (d, J=8.6 Hz, 2H), 7.68-7.56 (m, 5H), 7.29-7.18 (m, 3H), 7.08 (d, J=8.1 Hz, 1H), 5.67 (s, 2H), 3.83 (s, 2H), 3.33-3.23 (m, 2H), 3.17 (s, 3H), 2.39 (s, 3H), 1.09 (t, J=7.2 Hz, 3H). MS (ESI), m/z for C₃₂H₃₁N₃O₅S₂ ([M−H]⁻): Calcd 601.74, found 600.8.

### Example 51 2-(4-(ethylsulfonyl)phenyl)-N-(4-(6-methyl-1-(pyridin-2-ylmethyl)-1H-benzo[d]imidazol-2-yl)phenyl)acetamide

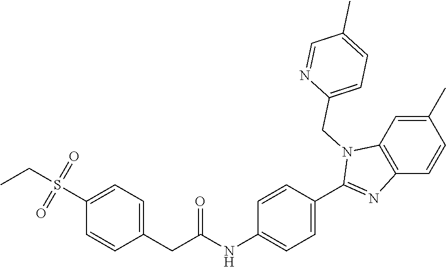 [SMILES: CCS(=O)(=O)c1ccc(CC(=O)Nc2ccc(-c3nc4ccc(C)cc4n3Cc3ccc(C)cn3)cc2)cc1]

A synthesis method was as that in Example 32, and a white solid was obtained with a yield of 41%. ¹H NMR (400 MHz, DMSO-d₆) δ 10.46 (s, 1H), 8.49 (d, J=4.4 Hz, 1H), 7.85 (d, J=8.1 Hz, 2H), 7.79-7.66 (m, 5H), 7.61 (d, J=8.1 Hz, 2H), 7.56 (d, J=8.2 Hz, 1H), 7.31-7.24 (m, 1H), 7.20 (s, 1H), 7.09 (d, J=7.8 Hz, 1H), 7.05 (d, J=8.2 Hz, 1H), 5.57 (s, 2H), 3.83 (s, 2H), 3.27 (q, J=7.2 Hz, 2H), 2.37 (s, 3H), 1.10 (t, J=7.3 Hz, 3H). MS (ESI), m/z for C₃₀H₂₈N₄O₃S ([M+H]⁺): Calcd 524.64, found 525.4.

### Example 52 N-(4-(1-benzyl-6-chloro-1H-benzo[d]imidazol-2-yl)phenyl)-2-(4-ethylsulfonyl) phenyl)acetamide

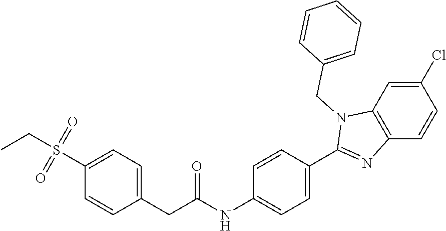 [SMILES: CCS(=O)(=O)c1ccc(CC(=O)Nc2ccc(-c3nc4ccc(Cl)cc4n3Cc3ccccc3)cc2)cc1]

A synthesis method was as that in Example 32, and a white solid was obtained with a yield of 37%. ¹H NMR (400 MHz, DMSO-d₆) δ 10.51 (s, 1H), 7.85 (d, J=8.3 Hz, 2H), 7.77-7.67 (m, 5H), 7.65-7.59 (m, 3H), 7.32-7.19 (m, 4H), 6.97 (d, J=7.1 Hz, 2H), 5.61 (s, 2H), 3.84 (s, 2H), 3.27 (q, J=7.3 Hz, 2H), 1.10 (t, J=7.4 Hz, 3H). MS (ESI), m/z for C₃₀H₂₆ClN₃O₃S ([M+H]⁺): Calcd 544.07, found 544.4.

### Example 53 N-(4-(1-benzyl-5-chloro-1H-benzo[d]imidazol-2-yl)phenyl)-2-(4-ethylsulfonyl) phenyl)acetamide

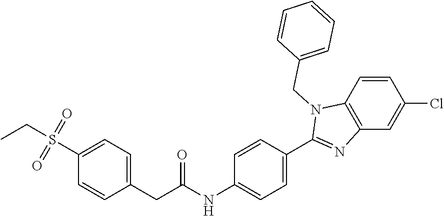 [SMILES: CCS(=O)(=O)c1ccc(CC(=O)Nc2ccc(-c3nc4cc(Cl)ccc4n3Cc3ccccc3)cc2)cc1]

A synthesis method was as that in Example 32, and a white solid was obtained with a yield of 35%. ¹H NMR (400 MHz, DMSO-d₆) δ 10.52 (s, 1H), 7.85 (d, J=8.1 Hz, 2H), 7.78-7.67 (m, 5H), 7.62 (d, J=8.1 Hz, 2H), 7.51 (d, J=8.6 Hz, 1H), 7.31-7.20 (m, 4H), 6.97 (d, J=7.0 Hz, 2H), 5.60 (s, 2H), 3.84 (s, 2H), 3.27 (q, J=7.2 Hz, 2H), 1.10 (t, J=7.3 Hz, 3H). MS (ESI), m/z for C₃₀H₂₆ClN₃O₃S ([M+H]⁺): Calcd 544.07, found 544.3. HPLC analysis: MeOH (1‰ NH₃·H₂O)—H₂O (75:25), t_{R}=17.64 min, 96.13% purity.

### Example 54 (S)-2-(4-(ethylsulfonyl)phenyl)-N-(4-(6-methyl-1-(1-(p-tolyl)ethyl)-1H-benzo[d]imidazol-2-yl)phenyl)acetamide

### Step 1: synthesis of (S)-4-(6-methyl-1-(1-(p-tolyl)ethyl)-1H-benzo[d]imidazol-2-yl)aniline

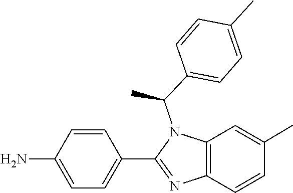 [SMILES: Cc1ccc([C@H](C)n2c(-c3ccc(N)cc3)nc3ccc(C)cc32)cc1]

3-fluoro-4-nitrotoluene and (S)-1-(4-methylphenyl)ethylamine were used as raw materials. For a synthesis method, reference was made to steps 1 to 4 in Example 31. A light yellow solid was obtained with a yield of 93%. ¹H NMR (400 MHz, DMSO-d₆) δ 7.47 (d, J=8.1 Hz, 1H), 7.31 (d, J=8.4 Hz, 2H), 7.14 (d, J=8.0 Hz, 2H), 7.05 (d, J=8.0 Hz, 2H), 6.94 (d, J=8.1 Hz, 1H), 6.88 (s, 1H), 6.66 (d, J=8.4 Hz, 2H), 5.80 (q, J=6.8 Hz, 1H), 5.54 (s, 2H), 2.27 (s, 3H), 2.26 (s, 3H), 1.90 (d, J=7.1 Hz, 3H).

### Step 2: synthesis of (S)-2-(4-(ethylsulfonyl)phenyl)-N-(4-(6-methyl-1-(1-(p-tolyl)ethyl)-1H-benzo[d]imidazol-2-yl)phenyl)acetamide

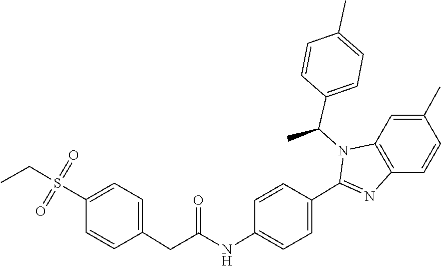 [SMILES: CCS(=O)(=O)c1ccc(CC(=O)Nc2ccc(-c3nc4ccc(C)cc4n3[C@@H](C)c3ccc(C)cc3)cc2)cc1]

2-(4-(ethylsulfonyl)phenyl)acetic acid (121.0 mg, 0.53 mmol), HOBT (89.2 mg, 0.66 mmol), EDCI (126.5 mg, 0.66 mol) and diisopropylethylamine (170.6 mg, 1.32 mmol) were dissolved in DCM (10 mL). The reaction mixture was stirred for 15 min, then the compound (S)-4-(6-methyl-1-(1-(p-tolyl)ethyl)-1H-benzo[d]imidazol-2-yl)aniline (150.2 mg, 0.44 mmol) was added to the reaction mixture, and the obtained mixture was stirred overnight at room temperature. After the reaction was finished, water was added to dilute the mixture and extracted with DCM (50 mL×3). Organic layers were combined, dried over anhydrous Na₂SO₄ and concentrated under reduced pressure. A crude product was isolated through silica gel column chromatography (PE:EA=1:1, v/v) to obtain a target compound as a white solid (53.4 mg with a yield of 22%). ¹H NMR (400 MHz, DMSO-d₆) δ 10.51 (s, 1H), 7.85 (d, J=8.1 Hz, 2H), 7.77 (d, J=8.5 Hz, 2H), 7.66-7.58 (m, 4H), 7.53 (d, J=8.1 Hz, 1H), 7.14 (d, J=7.8 Hz, 2H), 7.05 (d, J=7.9 Hz, 2H), 6.99 (d, J=8.3 Hz, 1H), 6.95 (s, 1H), 5.82-5.72 (m, 1H), 3.85 (s, 2H), 3.31-3.22 (m, 2H), 2.29 (s, 3H), 2.26 (s, 3H), 1.92 (d, J=7.0 Hz, 3H), 1.10 (t, J=7.3 Hz, 3H). MS (ESI), m/z for C₃₃H₃₃N₃O₃S ([M+H]⁺): Calcd 551.71, found 552.8.

### Example 55 Synthesis of 2-(4-(ethylsulfonyl)phenyl)-N-(4-(6-methyl-1-(pyridin-4-ylmethyl)-1H-benzo[d]imidazol-2-yl)phenyl)acetamide

### Step 1: synthesis of 5-methyl-2-nitro-N-(pyridin-4-ylmethyl)aniline

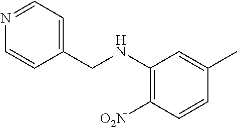 [SMILES: Cc1ccc([N+](=O)[O-])c(NCc2ccncc2)c1]

3-fluoro-4-nitrotoluene (2 g, 12.9 mmol), 4-picolylamine (1.68 g, 15.5 mmol) and potassium carbonate (2.68 g, 19.4 mmol) were dissolved in DMF (20 mL) and reacted for 1 h at 80° C. The reaction was monitored through TLC. After the reaction was finished, the reaction mixture was cooled to room temperature. Water was added to dilute the reaction mixture and extracted three times (50 mL×3) with ethyl acetate. The organic phases were combined, washed once with saturated brine, dried over anhydrous sodium sulfate, concentrated under reduced pressure and isolated through silica gel column chromatography (PE:EA=4:1, v/v) to obtain a target compound as a yellow solid (2.2 g with a yield of 70%). ¹H NMR (400 MHz, DMSO-d₆) δ 8.72 (t, J=6.2 Hz, 1H), 8.51 (d, J=5.9 Hz, 2H), 7.99 (d, J=8.7 Hz, 1H), 7.34 (d, J=5.5 Hz, 2H), 6.65 (s, 1H), 6.52 (dd, J=8.8, 0.8 Hz, 1H), 4.68 (d, J=6.4 Hz, 2H), 2.19 (s, 3H).

### Step 2: synthesis of 4-(6-methyl-1-(pyridin-4-ylmethyl)-1H-benzo[d]imidazol-2-yl)aniline

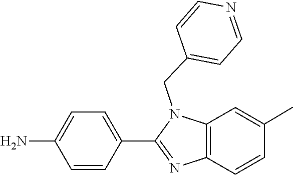 [SMILES: Cc1ccc2nc(-c3ccc(N)cc3)n(Cc3ccncc3)c2c1]

The intermediate 5-methyl-2-nitro-N-(pyridin-4-ylmethyl)aniline was used as a raw material. For a synthesis method, reference was made to steps 2 to 4 in Example 31. A yellow solid was obtained with a yield of 69%. ¹H NMR (400 MHz, DMSO-d₆) δ 8.49 (d, J=6.0 Hz, 2H), 7.53 (d, J=8.2 Hz, 1H), 7.32 (d, J=8.6 Hz, 2H), 7.15 (s, 1H), 7.04 (dd, J=8.2, 0.8 Hz, 1H), 6.98 (d, J=5.9 Hz, 2H), 6.60 (d, J=8.6 Hz, 2H), 5.60-5.47 (m, 4H), 2.37 (s, 3H).

### Step 3: synthesis of 2-(4-(ethylsulfonyl)phenyl)-N-(4-(6-methyl-1-(pyridin-4-ylmethyl)-1H-benzo[d]imidazol-2-yl)phenyl)acetamide

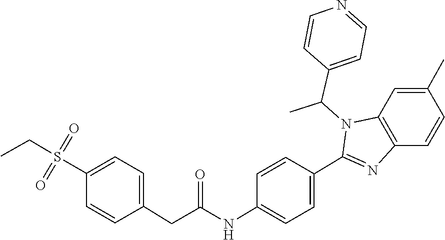 [SMILES: CCS(=O)(=O)c1ccc(CC(=O)Nc2ccc(-c3nc4ccc(C)cc4n3C(C)c3ccncc3)cc2)cc1]

The intermediate 4-(6-methyl-1-(pyridin-4-ylmethyl)-1H-benzo[d]imidazol-2-yl)aniline was used as a raw material. For a synthesis method, reference was made to step 2 in Example 54. A white solid was obtained with a yield of 26%. ¹H NMR (400 MHz, DMSO-d₆) δ 10.55 (s, 1H), 8.43 (m, 2H), 7.84 (d, J=8.3 Hz, 2H), 7.72 (d, J=8.7 Hz, 2H), 7.66-7.57 (m, 5H), 7.24 (s, 1H), 7.09 (dd, J=8.2, 0.8 Hz, 1H), 6.97-6.93 (m, 2H), 5.58 (s, 2H), 3.84 (s, 2H), 3.26 (q, J=7.6 Hz, 2H), 2.39 (s, 3H), 1.10 (t, J=7.3 Hz, 3H). MS (ESI), m/z for C₃₀H₂₈N₄O₃S ([M+H]⁺): Calcd 524.64, found 525.4.

### Example 56 2-(4-(ethylsulfonyl)phenyl)-N-(4-(6-methyl-1-(1-(p-tolyl)ethyl)-1H-benzo[d]imidazol-2-yl)phenyl)acetamide

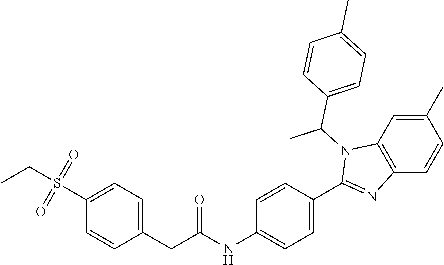 [SMILES: CCS(=O)(=O)c1ccc(CC(=O)Nc2ccc(-c3nc4ccc(C)cc4n3C(C)c3ccc(C)cc3)cc2)cc1]

A synthesis method was as that in Example 54, and a white solid was obtained with a yield of 25%. ¹H NMR (400 MHz, DMSO-d₆) δ 10.52 (s, 1H), 7.85 (d, J=8.2 Hz, 2H), 7.77 (d, J=8.6 Hz, 2H), 7.66-7.58 (m, 4H), 7.53 (d, J=8.2 Hz, 1H), 7.14 (d, J=8.0 Hz, 2H), 7.05 (d, J=8.0 Hz, 2H), 6.99 (d, J=8.4 Hz, 1H), 6.95 (s, 1H), 5.76 (q, J=6.8 Hz, 2H), 3.85 (s, 2H), 3.27 (q, J=7.2 Hz, 2H), 2.29 (s, 3H), 2.26 (s, 3H), 1.92 (d, J=7.1 Hz, 3H), 1.10 (t, J=7.3 Hz, 3H). MS (ESI), m/z for C₃₃H₃₃N₃O₃S ([M+H]⁺): Calcd 551.71, found 552.6.

### Example 57 (R)-2-(4-(ethylsulfonyl)phenyl)-N-(4-(6-methyl-1-(1-(p-tolyl)ethyl)-1H-benzo[d]imidazol-2-yl)phenyl)acetamide

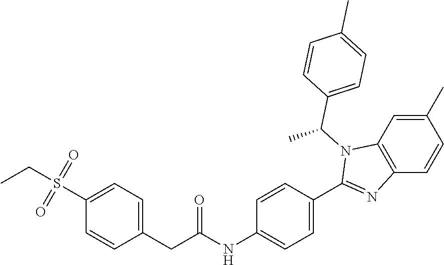 [SMILES: CCS(=O)(=O)c1ccc(CC(=O)Nc2ccc(-c3nc4ccc(C)cc4n3[C@H](C)c3ccc(C)cc3)cc2)cc1]

A synthesis method was as that in Example 54, and a white solid was obtained with a yield of 18%. ¹H NMR (400 MHz, DMSO-d₆) δ 10.52 (s, 1H), 7.85 (d, J=8.2 Hz, 2H), 7.77 (d, J=8.5 Hz, 2H), 7.66-7.58 (m, 4H), 7.53 (d, J=8.2 Hz, 1H), 7.14 (d, J=8.0 Hz, 2H), 7.05 (d, J=8.0 Hz, 2H), 6.99 (d, J=8.2 Hz, 1H), 6.95 (s, 1H), 5.76 (q, J=6.8 Hz, 1H), 3.85 (s, 2H), 3.27 (q, J=7.2 Hz, 2H), 2.29 (s, 3H), 2.26 (s, 3H), 1.92 (d, J=7.1 Hz, 3H), 1.10 (t, J=7.4 Hz, 3H). MS (ESI), m/z for C₃₃H₃₃N₃O₃S ([M+H]⁺): Calcd 551.71, found 552.7.

### Example 58 2-(4-(ethylsulfonyl)phenyl)-N-(4-(1-(4-fluorophenethyl)-6-methyl-1H-benzo[d]imidazol-2-yl)phenyl)acetamide

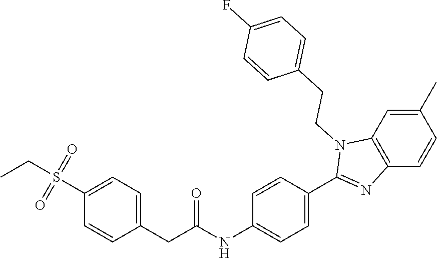 [SMILES: CCS(=O)(=O)c1ccc(CC(=O)Nc2ccc(-c3nc4ccc(C)cc4n3CCc3ccc(F)cc3)cc2)cc1]

A synthesis method was as that in Example 54, and a white solid was obtained with a yield of 29%. ¹H NMR (400 MHz, DMSO-d₆) δ 10.47 (s, 1H), 7.87 (d, J=8.4 Hz, 2H), 7.72 (d, J=8.7 Hz, 2H), 7.64 (d, J=8.4 Hz, 2H), 7.53-7.47 (m, 3H), 7.43 (s, 1H), 7.05 (dd, J=8.2, 0.8 Hz, 1H), 6.99-6.94 (m, 4H), 4.44 (t, J=7.3 Hz, 2H), 3.86 (s, 2H), 3.31-3.23 (m, 2H), 2.98 (t, J=7.4 Hz, 2H), 2.46 (s, 3H), 1.11 (t, J=7.4 Hz, 3H). MS (ESI), m/z for C₃₂H₃₀FN₃O₃S ([M−H]⁻): Calcd 555.67, found 554.7.

### Example 59 2-(4-(ethylsulfonyl)phenyl)-N-(4-(6-methyl-1-(4-(trifluoromethyl)benzyl)-1H-benzo[d]imidazol-2-yl)phenyl)acetamide

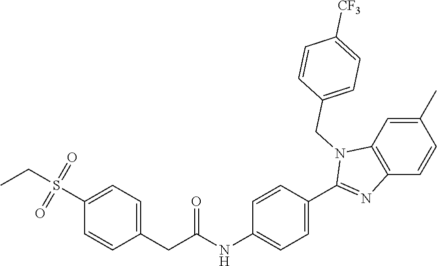 [SMILES: CCS(=O)(=O)c1ccc(CC(=O)Nc2ccc(-c3nc4ccc(C)cc4n3Cc3ccc(C(F)(F)F)cc3)cc2)cc1]

A synthesis method was as that in Example 54, and a white solid was obtained with a yield of 23%. ¹H NMR (400 MHz, DMSO-d₆) δ 10.48 (s, 1H), 7.85 (d, J=8.1 Hz, 2H), 7.75-7.54 (m, 9H), 7.26 (s, 1H), 7.18 (d, J=8.1 Hz, 2H), 7.08 (d, J=8.1 Hz, 1H), 5.65 (s, 2H), 3.83 (s, 2H), 3.32-3.22 (m, 2H), 2.39 (s, 3H), 1.09 (t, J=7.3 Hz, 3H). MS (ESI), m/z for C₃₂H₂₈F₃N₃O₃S ([M−H]⁻): Calcd 591.65, found 590.3.

### Example 60 4-((2-(4-(2-(4-(ethylsulfonyl)phenyl)acetylamino)phenyl)-6-methyl-1H-benzo[d]imidazol-1-yl)methyl)methyl benzoate

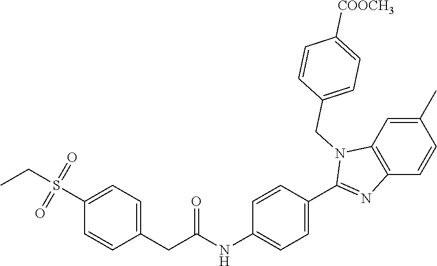 [SMILES: CCS(=O)(=O)c1ccc(CC(=O)Nc2ccc(-c3nc4ccc(C)cc4n3Cc3ccc(C(=O)OC)cc3)cc2)cc1]

A synthesis method was as that in Example 54, and a white solid was obtained with a yield of 29%. ¹H NMR (400 MHz, DMSO-d₆) δ 10.48 (s, 1H), 7.88 (d, J=8.2 Hz, 2H), 7.84 (d, J=8.3 Hz, 2H), 7.70 (d, J=8.7 Hz, 2H), 7.66-7.56 (m, 5H), 7.24 (s, 1H), 7.12 (d, J=8.2 Hz, 2H), 7.08 (dd, J=8.2, 0.4 Hz, 1H), 5.63 (s, 2H), 3.83 (s, 2H), 3.81 (s, 3H), 3.27 (q, J=7.2 Hz, 2H), 2.38 (s, 3H), 1.09 (t, J=7.3 Hz, 3H). MS (ESI), m/z for C₃₃H₃₁N₃O₅S ([M+H]⁺): Calcd 581.69, found 582.4.

### Example 61 4-((2-(4-(2-(4-(ethylsulfonyl)phenyl)acetylamino)phenyl)-6-methyl-1H-benzo[d]imidazol-1-yl)methyl)benzoic acid

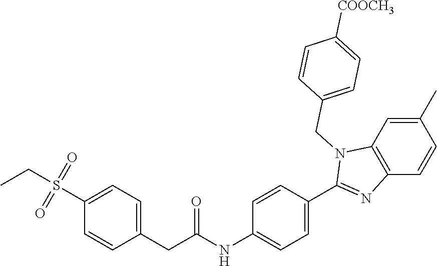 [SMILES: CCS(=O)(=O)c1ccc(CC(=O)Nc2ccc(-c3nc4ccc(C)cc4n3Cc3ccc(C(=O)OC)cc3)cc2)cc1]

Example 60 was used as a raw material. For a synthesis method, reference was made to Example 19. A white solid was obtained with a yield of 69%. ¹H NMR (500 MHz, DMSO) δ 10.78 (s, 1H), 7.87 (d, J=8.2 Hz, 2H), 7.84 (d, J=8.2 Hz, 2H), 7.81 (d, J=8.3 Hz, 2H), 7.74-7.66 (m, 3H), 7.62 (d, J=7.9 Hz, 2H), 7.43 (s, 1H), 7.26 (d, J=7.9 Hz, 1H), 7.18 (d, J=8.0 Hz, 2H), 5.71 (s, 2H), 3.87 (s, 2H), 3.27 (q, J=7.5 Hz, 2H), 2.42 (s, 3H), 1.09 (t, J=7.3 Hz, 3H). MS (ESI), m/z for C₃₂H₂₉N₃O₅S ([M+H]⁺): Calcd 567.66, found 568.7.

### Example 62 2-(4-(ethylsulfonyl)phenyl)-N-(4-(6-methyl-1-(pyridin-3-ylmethyl)-1H-benzo[d]imidazol-2-yl)phenyl)acetamide

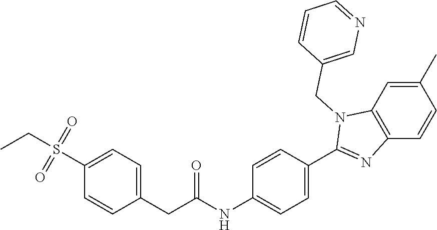 [SMILES: CCS(=O)(=O)c1ccc(CC(=O)Nc2ccc(-c3nc4ccc(C)cc4n3Cc3cccnc3)cc2)cc1]

A synthesis method was as that in Example 54, and a white solid was obtained with a yield of 44%. ¹H NMR (400 MHz, DMSO-d₆) δ 10.51 (s, 1H), 8.46-8.40 (t, J=2.8 Hz, 1H), 8.26 (s, 1H), 7.85 (d, J=8.1 Hz, 2H), 7.74 (d, J=8.5 Hz, 2H), 7.67 (d, J=8.6 Hz, 2H), 7.62 (d, J=8.1 Hz, 2H), 7.58 (d, J=8.3 Hz, 1H), 7.35 (s, 1H), 7.31-7.25 (m, 2H), 7.08 (d, J=8.1 Hz, 1H), 5.61 (s, 2H), 3.85 (s, 2H), 3.28 (q, J=7.6 Hz, 2H), 2.40 (s, 3H), 1.10 (t, J=7.3 Hz, 3H). MS (ESI), m/z for C₃₀H₂₈N₄O₃S ([M+H]⁺): Calcd 524.64, found 525.4.

### Example 63 (R)-2-(4-(ethylsulfonyl)phenyl)-N-(4-(1-(1-(p-tolyl)ethyl)-6-(trifluoromethyl)-1H-benzo[d]imidazol-2-yl)phenyl)acetamide

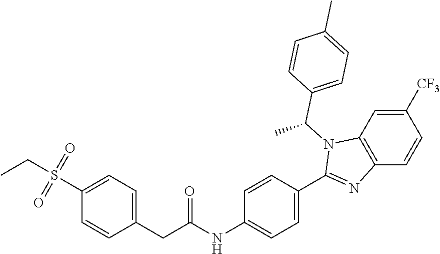 [SMILES: CCS(=O)(=O)c1ccc(CC(=O)Nc2ccc(-c3nc4ccc(C(F)(F)F)cc4n3[C@H](C)c3ccc(C)cc3)cc2)cc1]

A synthesis method was as that in Example 54, and a white solid was obtained with a yield of 44%. ¹H NMR (400 MHz, DMSO-d₆) δ 10.57 (s, 1H), 7.89-7.84 (m, 3H), 7.82 (d, J=8.5 Hz, 2H), 7.69 (d, J=8.3 Hz, 2H), 7.63 (d, J=7.9 Hz, 2H), 7.50 (d, J=8.5 Hz, 1H), 7.37 (s, 1H), 7.16 (d, J=7.8 Hz, 2H), 7.10 (d, J=7.8 Hz, 2H), 5.95-5.83 (m, 1H), 3.86 (s, 2H), 3.31-3.22 (m, 2H), 2.26 (s, 3H), 1.94 (d, J=6.9 Hz, 3H), 1.10 (t, J=7.3 Hz, 3H). MS (ESI), m/z for C₃₃H₃₀F3N₃O₃S ([M−H]⁻): Calcd 605.68, found 604.4.

### Example 64 (R)-2-(4-(ethylsulfonyl)phenyl)-N-(4-(6-methoxy-1-(1-(p-tolyl)ethyl)-1H-benzo[d]imidazol-2-yl)phenyl)acetamide

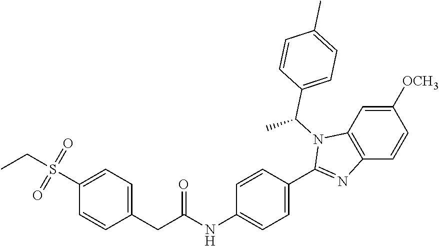 [SMILES: CCS(=O)(=O)c1ccc(CC(=O)Nc2ccc(-c3nc4ccc(OC)cc4n3[C@H](C)c3ccc(C)cc3)cc2)cc1]

A synthesis method was as that in Example 54, and a white solid was obtained with a yield of 27%. ¹H NMR (400 MHz, DMSO-d₆) δ 10.53 (s, 1H), 7.86 (d, J=8.2 Hz, 2H), 7.78 (d, J=8.6 Hz, 2H), 7.66-7.59 (m, 4H), 7.54 (d, J=8.8 Hz, 1H), 7.15 (d, J=8.0 Hz, 2H), 7.09 (d, J=8.0 Hz, 2H), 6.81 (dd, J=8.8, 2.2 Hz, 1H), 6.53 (d, J=1.9 Hz, 1H), 5.76 (q, J=6.8 Hz, 1H), 3.85 (s, 2H), 3.62 (s, 3H), 3.28 (q, J=7.4 Hz, 2H), 2.26 (s, 3H), 1.91 (d, J=7.1 Hz, 3H), 1.10 (t, J=7.3 Hz, 3H). MS (ESI), m/z for C₃₃H₃₃N₃O₄S ([M−H]⁻): Calcd 567.70, found 566.4.

### Example 65 (R)-2-(4-(ethylsulfonyl)phenyl)-N-(4-(5-methyl-1-(1-(p-tolyl)ethyl)-1H-benzo[d]imidazol-2-yl)phenyl)acetamide

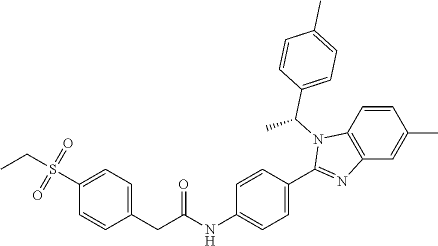 [SMILES: CCS(=O)(=O)c1ccc(CC(=O)Nc2ccc(-c3nc4cc(C)ccc4n3[C@H](C)c3ccc(C)cc3)cc2)cc1]

A synthesis method was as that in Example 54, and a white solid was obtained with a yield of 31%. ¹H NMR (400 MHz, DMSO-d₆) δ 10.54 (s, 1H), 7.85 (d, J=8.3 Hz, 2H), 7.78 (d, J=8.6 Hz, 2H), 7.67-7.60 (m, 4H), 7.44 (s, 1H), 7.12 (d, J=8.0 Hz, 2H), 7.04 (d, J=8.0 Hz, 2H), 6.98 (d, J=8.3 Hz, 1H), 6.88 (d, J=8.2 Hz, 1H), 5.76 (q, J=7.1 Hz, 1H), 3.85 (s, 2H), 3.28 (q, J=7.4 Hz, 2H), 2.35 (s, 3H), 2.24 (s, 3H), 1.92 (d, J=7.0 Hz, 3H), 1.09 (t, J=7.3 Hz, 3H). MS (ESI), m/z for C₃₃H₃₃N₃O₃S ([M−H]⁻): Calcd 551.71, found 550.4.

### Example 66 (R)-2-(4-(ethylsulfonyl)phenyl)-N-(4-(5-methoxy-1-(1-(p-tolyl)ethyl)-1H-benzo[d]imidazol-2-yl)phenyl)acetamide (27f)

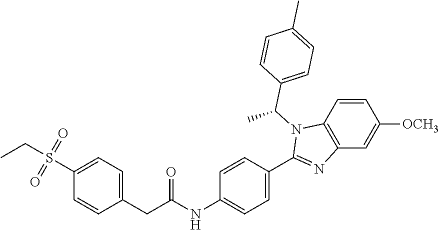 [SMILES: CCS(=O)(=O)c1ccc(CC(=O)Nc2ccc(-c3nc4cc(OC)ccc4n3[C@H](C)c3ccc(C)cc3)cc2)cc1]

A synthesis method was as that in Example 54, and a white solid was obtained with a yield of 25%. ¹H NMR (400 MHz, DMSO-d₆) δ 10.53 (s, 1H), 7.85 (d, J=7.8 Hz, 2H), 7.78 (d, J=8.2 Hz, 2H), 7.69-7.58 (m, 4H), 7.17 (s, 1H), 7.12 (d, J=7.5 Hz, 2H), 7.05 (d, J=7.5 Hz, 2H), 6.98 (d, J=8.9 Hz, 1H), 6.70 (d, J=8.8 Hz, 1H), 5.82-5.69 (m, 1H), 3.85 (s, 2H), 3.75 (s, 3H), 3.33-3.22 (m, 2H), 2.25 (s, 3H), 1.91 (d, J=6.7 Hz, 3H), 1.10 (t, J=7.2 Hz, 3H). MS (ESI), m/z for C₃₃H₃₃N₃O₄S ([M+H]⁺): Calcd 567.70, found 568.8.

### Example 67 (R)-2-(4-(ethylsulfonyl)phenyl)-N-(4-(1-(1-(4-fluorophenyl)ethyl)-6-(trifluoromethyl)-1H-benzo[d]imidazol-2-yl)phenyl)acetamide

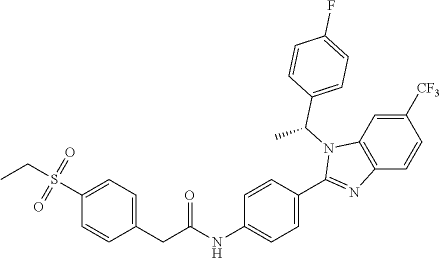 [SMILES: CCS(=O)(=O)c1ccc(CC(=O)Nc2ccc(-c3nc4ccc(C(F)(F)F)cc4n3[C@H](C)c3ccc(F)cc3)cc2)cc1]

A synthesis method was as that in Example 54, and a white solid was obtained with a yield of 39%. ¹H NMR (500 MHz, DMSO-d₆) δ 10.58 (s, 1H), 7.91-7.84 (m, 3H), 7.82 (d, J=7.9 Hz, 2H), 7.69 (d, J=8.1 Hz, 2H), 7.63 (d, J=7.5 Hz, 2H), 7.51 (d, J=8.1 Hz, 1H), 7.35 (s, 1H), 7.30-7.23 (m, 2H), 7.22-7.15 (m, 2H), 5.96-5.96 (m, 1H), 3.86 (s, 2H), 3.32-3.23 (m, 2H), 1.96 (d, J=6.5 Hz, 3H), 1.10 (t, J=7.1 Hz, 3H). MS (ESI), m/z for C₃₂H₂₇F₄N₃O₃S ([M−H]⁻): Calcd 609.64, found 608.2. HPLC analysis: MeOH—H₂O (85:15), t_{R}=13.02 min, 98.08% purity.

### Example 68 (S)-2-(4-(ethylsulfonyl)phenyl)-N-(4-(1-(1-(4-fluorophenyl)ethyl)-6-(trifluoromethyl)-1H-benzo[d]imidazol-2-yl)phenyl)acetamide

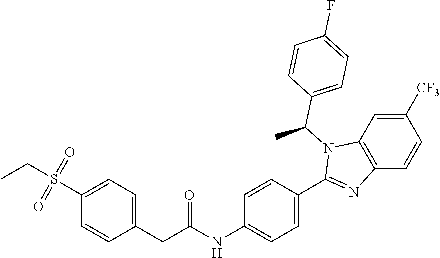 [SMILES: CCS(=O)(=O)c1ccc(CC(=O)Nc2ccc(-c3nc4ccc(C(F)(F)F)cc4n3[C@@H](C)c3ccc(F)cc3)cc2)cc1]

A synthesis method was as that in Example 54, and a white solid was obtained with a yield of 28%. ¹H NMR (500 MHz, DMSO-d₆) δ 10.58 (s, 1H), 7.90-7.83 (m, 3H), 7.82 (d, J=8.6 Hz, 2H), 7.69 (d, J=8.6 Hz, 2H), 7.63 (d, J=8.2 Hz, 2H), 7.51 (d, J=8.1 Hz, 1H), 7.35 (s, 1H), 7.29-7.24 (m, 2H), 7.18 (t, J=8.8 Hz, 2H), 5.92 (q, J=6.8 Hz, 1H), 3.86 (s, 2H), 3.28 (q, J=7.4 Hz, 2H), 1.96 (d, J=7.1 Hz, 3H), 1.10 (t, J=7.3 Hz, 3H). MS (ESI), m/z for C₃₂H₂₇F₄N₃O₃S ([M−H]⁻): Calcd 609.64, found 608.3.

### Example 69 (R)-2-(4-(2-(4-(ethylsulfonyl)phenyl)acetylamino)phenyl)-N-isopropyl-1-(1-(p-tolyl)ethyl)-1H-benzo[d]imidazol-6-formamide

### Step 1: Synthesis of 3-fluoro-N-isopropyl-4-nitrobenzamide

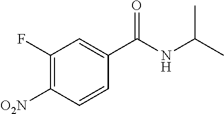 [SMILES: CC(C)NC(=O)c1ccc([N+](=O)[O-])c(F)c1]

3-Fluoro-4-nitrobenzoic acid and isopropylamine were used as raw materials. For a synthesis method, reference was made to step 5 in Example 31. A yellow solid was obtained with a yield of 95%. ¹H NMR (400 MHz, DMSO-d₆) δ 8.59 (d, J=7.2 Hz, 1H), 8.25 (t, J=8.1 Hz, 1H), 7.97 (dd, J=12.1, 1.5 Hz, 1H), 7.87 (d, J=8.5 Hz, 1H), 4.14-4.02 (m, 1H), 1.18 (d, J=6.6 Hz, 6H).

### Step 2: synthesis of (R)-2-(4-(2-(4-(ethylsulfonyl)phenyl)acetylamino)phenyl)-N-isopropyl-1-(1-(p-tolyl)ethyl)-1H-benzo[d]imidazol-6-formamide

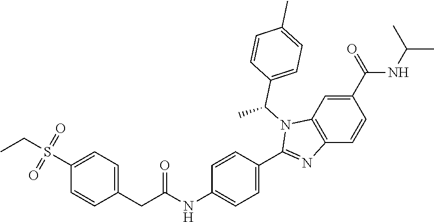 [SMILES: CCS(=O)(=O)c1ccc(CC(=O)Nc2ccc(-c3nc4ccc(C(=O)NC(C)C)cc4n3[C@H](C)c3ccc(C)cc3)cc2)cc1]

3-Fluoro-N-isopropyl-4-nitrobenzamide and (R)-1-(4-methylphenyl)ethylamine were used as raw materials. For a synthesis method, reference was made to Example 53. A white solid was obtained with a yield of 26%. ¹H NMR (400 MHz, DMSO-d₆) δ 10.57 (s, 1H), 8.14 (d, J=7.8 Hz, 1H), 7.85 (d, J=8.3 Hz, 2H), 7.79 (d, J=8.7 Hz, 2H), 7.76-7.60 (m, 7H), 7.14 (d, J=8.1 Hz, 2H), 7.05 (d, J=8.0 Hz, 2H), 5.88-5.80 (m, 1H), 4.10-4.01 (m, 1H), 3.86 (s, 2H), 3.32-3.22 (m, 2H), 2.25 (s, 3H), 1.97 (d, J=7.1 Hz, 3H), 1.14 (d, J=6.5 Hz, 6H), 1.10 (t, J=7.4 Hz, 3H). MS (ESI), m/z for C₃₆H₃₈N₄O₄S ([M−H]⁻): Calcd 622.78, found 621.0.

### Example 70 (R)—N-cyclopentyl-2-(4-(2-(4-(ethylsulfonyl)phenyl)acetylamino)phenyl)-1-(1-(p-tolyl)ethyl)-1H-benzo[d]imidazol-6-formamide

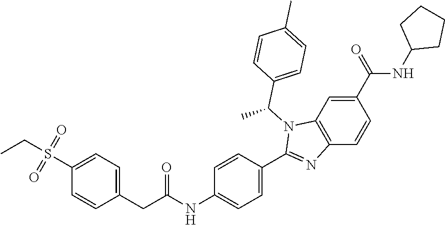 [SMILES: CCS(=O)(=O)c1ccc(CC(=O)Nc2ccc(-c3nc4ccc(C(=O)NC5CCCC5)cc4n3[C@H](C)c3ccc(C)cc3)cc2)cc1]

A synthesis method was as that in Example 69, and a white solid was obtained with a yield of 12%. ¹H NMR (400 MHz, DMSO-d₆) δ 10.55 (s, 1H), 8.19 (d, J=7.0 Hz, 1H), 7.85 (d, J=8.3 Hz, 2H), 7.79 (d, J=8.3 Hz, 2H), 7.76-7.57 (m, 7H), 7.14 (d, J=7.3 Hz, 2H), 7.05 (d, J=7.7 Hz, 2H), 5.89-5.78 (m, 1H), 4.23-4.12 (m, 1H), 3.85 (s, 2H), 3.33-3.22 (m, 2H), 2.25 (s, 3H), 1.97 (d, J=7.0 Hz, 3H), 1.92-1.79 (m, 2H), 1.75-1.62 (m, 2H), 1.59-1.44 (m, 4H), 1.09 (t, J=7.3 Hz, 3H). MS (ESI), m/z for C₃₈H₄₀N₄O₄S ([M+H]⁺): Calcd 648.82, found 650.0.

### Example 71 (R)-2-(4-(2-(4-(ethylsulfonyl)phenyl)acetylamino)phenyl)-N-isopropyl-1-(1-(p-tolyl)ethyl)-1H-benzo[d]imidazol-5-formamide

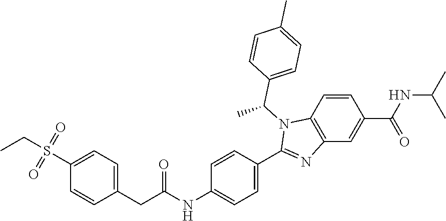 [SMILES: CCS(=O)(=O)c1ccc(CC(=O)Nc2ccc(-c3nc4cc(C(=O)NC(C)C)ccc4n3[C@H](C)c3ccc(C)cc3)cc2)cc1]

A synthesis method was as that in Example 69, and a white solid was obtained with a yield of 29%. ¹H NMR (400 MHz, DMSO-d₆) δ 10.57 (s, 1H), 8.18 (s, 1H), 8.14 (d, J=6.7 Hz, 1H), 7.86 (d, J=7.6 Hz, 2H), 7.81 (d, J=7.7 Hz, 2H), 7.68 (d, J=7.3 Hz, 2H), 7.63 (d, J=7.5 Hz, 2H), 7.57 (d, J=7.9 Hz, 1H), 7.17-7.09 (m, 3H), 7.08-7.00 (m, 2H), 5.88-5.74 (m, 1H), 4.16-4.02 (m, 1H), 3.86 (s, 2H), 3.31-3.22 (d, J=7.1 Hz, 2H), 2.24 (s, 3H), 1.96 (d, J=4.8 Hz, 3H), 1.16 (d, J=5.5 Hz, 6H), 1.13-1.07 (m, 3H). MS (ESI), m/z for C₃₆H₃₈N₄O₄S ([M+H]⁺): Calcd 622.78, found 623.3.

### Example 72 (R)—N-cyclopentyl-2-(4-(2-(4-(ethylsulfonyl)phenyl)acetylamino)phenyl)-1-(1-(p-tolyl)ethyl)-1H-benzo[d]imidazol-5-formamide

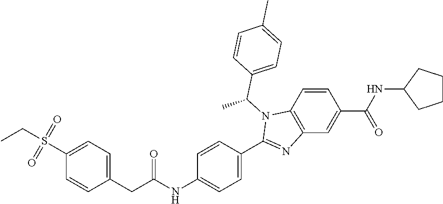 [SMILES: CCS(=O)(=O)c1ccc(CC(=O)Nc2ccc(-c3nc4cc(C(=O)NC5CCCC5)ccc4n3[C@H](C)c3ccc(C)cc3)cc2)cc1]

A synthesis method was as that in Example 69, and a white solid was obtained with a yield of 11%. ¹H NMR (400 MHz, DMSO-d₆) δ 10.56 (s, 1H), 8.24-8.15 (m, 2H), 7.85 (d, J=8.1 Hz, 2H), 7.80 (d, J=8.6 Hz, 2H), 7.67 (d, J=8.6 Hz, 2H), 7.62 (d, J=8.1 Hz, 2H), 7.56 (d, J=8.5 Hz, 1H), 7.16-7.09 (m, 3H), 7.04 (d, J=7.9 Hz, 2H), 5.86-5.76 (m, 1H), 4.28-4.17 (m, 1H), 3.86 (s, 2H), 3.33-3.23 (m, 2H), 2.24 (s, 3H), 1.96 (d, J=6.9 Hz, 3H), 1.92-1.80 (m, 2H), 1.76-1.64 (m, 2H), 1.58-1.48 (m, 4H), 1.09 (t, J=7.3 Hz, 3H). MS (ESI), m/z for C₃₈H₄₀N₄O₄S ([M+H]⁺): Calcd 648.82, found 649.9.

### Example 73 2-acetylamino-2-(4-(ethylsulfonyl)phenyl)-N-(4-(6-methyl-1-((S)-1-(p-tolyl) ethyl)-1H-benzo[d]imidazol-2-yl)phenyl)acetamide

### Step 1: synthesis of tert-butyl(1-(4-(ethylsulfonyl)phenyl)-2-((4-(6-methyl-1-((S)-1-(p-tolyl)ethyl)-1H-benzo[d]imidazol-2-yl)phenyl)amino)-2-oxoethyl)carbamate

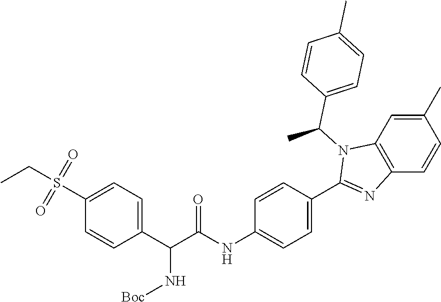 [SMILES: CCS(=O)(=O)c1ccc(C(NC(=O)OC(C)(C)C)C(=O)Nc2ccc(-c3nc4ccc(C)cc4n3[C@@H](C)c3ccc(C)cc3)cc2)cc1]

(S)-4-(6-methyl-1-(1-(p-tolyl)ethyl)-1H-benzo[d]imidazol-2-yl)aniline and 2-((tert-butyloxycarbonyl)amino)-2-(4-(ethylsulfonyl)phenyl)acetic acid were used as raw materials. For a synthesis method, reference was made to step 5 in Example 31. A white solid was obtained with a yield of 46%. ¹H NMR (400 MHz, DMSO-d₆) δ 10.62 (s, 1H), 7.89 (d, J=8.2 Hz, 2H), 7.82-7.69 (m, 5H), 7.60 (d, J=8.4 Hz, 2H), 7.52 (d, J=8.2 Hz, 1H), 7.12 (d, J=7.9 Hz, 2H), 7.04 (d, J=7.9 Hz, 2H), 6.98 (d, J=8.3 Hz, 1H), 6.94 (s, 1H), 5.74 (q, J=7.2 Hz, 1H), 5.52 (d, J=7.2 Hz, 1H), 3.31-3.20 (m, 2H), 2.28 (s, 3H), 2.25 (s, 3H), 1.90 (d, J=7.0 Hz, 3H), 1.40 (s, 9H), 1.08 (t, J=7.3 Hz, 3H).

### Step 2: synthesis of 2-amino-2-(4-(ethylsulfonyl)phenyl)-N-(4-(6-methyl-1-((S)-1-(p-tolyl) ethyl)-1H-benzo[d]imidazopyridin-2-yl)phenyl)acetamide

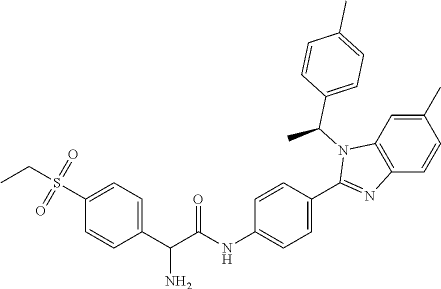 [SMILES: CCS(=O)(=O)c1ccc(C(N)C(=O)Nc2ccc(-c3nc4ccc(C)cc4n3[C@@H](C)c3ccc(C)cc3)cc2)cc1]

Tert-butyl(1-(4-(ethylsulfonyl)phenyl)-2-((4-(6-methyl-1-((S)-1-(p-tolyl)ethyl)-1H-benzo[d]imidazol-2-yl)phenyl)amino)-2-oxoethyl)carbamate (190 mg, 0.28 mmol) was dissolved in DCM (10 mL). TFA (5 mL) was added dropwise to the mixture and reacted for 3 h at room temperature. After the reaction was finished, the organic solvent was removed, and a mixed solvent of ethyl acetate and petroleum ether was recrystallized to obtain a target product as a white solid (130 mg with a yield of 82%). MS (ESI), m/z for C₃₃H₃₄N₄O₃S ([M+H]⁺): Calcd 566.72, found 567.5.

### Step 3: synthesis of 2-acetylamino-2-(4-(ethylsulfonyl)phenyl)-N-(4-(6-methyl-1-((S)-1-(p-tolyl)ethyl)-1H-benzo[d]imidazol-2-yl)phenyl)acetamide

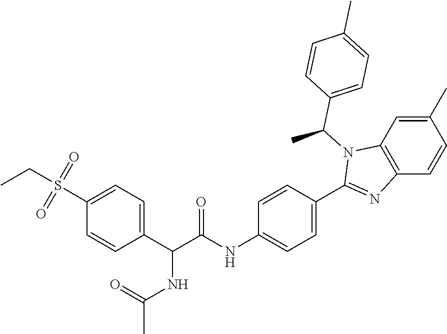 [SMILES: CCS(=O)(=O)c1ccc(C(NC(C)=O)C(=O)Nc2ccc(-c3nc4ccc(C)cc4n3[C@@H](C)c3ccc(C)cc3)cc2)cc1]

2-Amino-2-(4-(ethylsulfonyl)phenyl)-N-(4-(6-methyl-1-((S)-1-(p-tolyl)ethyl)-1H-benzo[d]imidazopyridin-2-yl)phenyl)acetamide (130 mg, 0.23 mmol) was dissolved in DCM (10 mL), then triethylamine (23.3 mg, 0.23 mmol) and acetic anhydride (23.5 mg, 0.23 mmol) were added to the mixture, and the mixture was stirred to react for 3 h at room temperature. After the reaction was finished, water was added to dilute the mixture and extracted with DCM (50 mL×3). Organic layers were combined, dried over anhydrous Na₂SO₄ and concentrated under reduced pressure. A crude product was isolated through silica gel column chromatography (PE:EA=1:4, v/v) to obtain a target compound as a white solid (86.8 mg with a yield of 62%). ¹H NMR (400 MHz, DMSO-d₆) δ 10.74 (s, 1H), 8.89 (d, J=7.8 Hz, 1H), 7.91 (d, J=8.3 Hz, 2H), 7.80-7.73 (m, 4H), 7.61 (d, J=8.6 Hz, 2H), 7.53 (d, J=8.2 Hz, 1H), 7.13 (d, J=8.1 Hz, 2H), 7.05 (d, J=8.0 Hz, 2H), 6.99 (d, J=8.5 Hz, 1H), 6.94 (s, 1H), 5.82 (d, J=7.8 Hz, 1H), 5.75 (q, J=6.9 Hz, 1H), 3.31-3.23 (m, 2H), 2.29 (s, 3H), 2.25 (s, 3H), 1.96 (s, 3H), 1.91 (d, J=7.1 Hz, 3H), 1.09 (t, J=7.3 Hz, 3H). MS (ESI), m/z for C₃₅H₃₆N₄O₄S ([M+H]⁺): Calcd 608.76, found 609.8.

Isomer 1 (Example 74) and Isomer 2 (Example 75) of Example 73

Example 73 (2.72 g) was purified through supercritical fluid chromatography (SFC) chiral separation (Chiralpak IC, 0.46 cm ID×15 cm L, 214 nm, hexane/ethanol=30/70 (V/V), 1 mL/min, 35° C.) to obtain two corresponding isomers: Isomer 1 (Example 74) (Peak 1, 1.25 g, >98% ee, white solid) and Isomer 2 (Example 75) (Peak 2, 1.31 g, >98% ee, white solid). Note: no single crystal of the compound was available at present, and no absolute configuration of the compound can be determined.

Example 74: Isomer 1 (R or S)¹H NMR (500 MHz, DMSO-d₆) δ 10.75 (s, 1H), 8.91 (d, J=7.7 Hz, 1H), 7.91 (d, J=7.6 Hz, 2H), 7.79-7.73 (m, 4H), 7.61 (d, J=7.8 Hz, 2H), 7.53 (d, J=8.2 Hz, 1H), 7.13 (d, J=7.6 Hz, 2H), 7.04 (d, J=7.6 Hz, 2H), 6.99 (d, J=8.1 Hz, 1H), 6.94 (s, 1H), 5.82 (d, J=7.7 Hz, 1H), 5.74 (q, J=6.2 Hz, 1H), 3.28 (q, J=7.4 Hz, 2H), 2.29 (s, 3H), 2.25 (s, 3H), 1.95 (s, 3H), 1.91 (d, J=7.0 Hz, 3H), 1.09 (t, J=7.9 Hz, 3H). MS (ESI), m/z for C₃₅H₃₆N₄O₄S ([M+H]⁺): Calcd 608.76, found 609.8.

Example 75: Isomer 2 (S or R)¹H NMR (500 MHz, DMSO-d₆) δ 10.75 (s, 1H), 8.91 (d, J=7.8 Hz, 1H), 7.91 (d, J=7.7 Hz, 2H), 7.79-7.74 (m, 4H), 7.61 (d, J=8.0 Hz, 2H), 7.53 (d, J=8.1 Hz, 1H), 7.13 (d, J=7.6 Hz, 2H), 7.04 (d, J=7.6 Hz, 2H), 6.99 (d, J=8.2 Hz, 1H), 6.94 (s, 1H), 5.82 (d, J=7.6 Hz, 1H), 5.74 (q, J=6.3 Hz, 1H), 3.28 (q, J=7.5 Hz, 2H), 2.29 (s, 3H), 2.25 (s, 3H), 1.95 (s, 3H), 1.91 (d, J=7.0 Hz, 3H), 1.09 (t, J=7.3 Hz, 3H). MS (ESI), m/z for C₃₅H₃₆N₄O₄S ([M+H]⁺): Calcd 608.76, found 609.8.

### Example 76 2-acetylamino-2-(4-(ethylsulfonyl)phenyl)-N-(4-(6-methyl-1-pentyl-1H-benzo[d]imidazol-2-yl)phenyl)acetamide

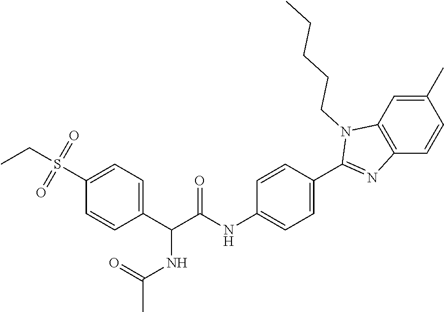 [SMILES: CCCCCn1c(-c2ccc(NC(=O)C(NC(C)=O)c3ccc(S(=O)(=O)CC)cc3)cc2)nc2ccc(C)cc21]

A synthesis method was as that in Example 73, and a white solid was obtained with a yield of 47%. ¹H NMR (400 MHz, DMSO-d₆) δ 10.72 (s, 1H), 8.89 (d, J=7.6 Hz, 1H), 7.93 (d, J=8.1 Hz, 2H), 7.81-7.74 (m, 4H), 7.70 (d, J=8.4 Hz, 2H), 7.51 (d, J=8.1 Hz, 1H), 7.40 (s, 1H), 7.04 (d, J=8.1 Hz, 1H), 5.83 (d, J=7.6 Hz, 1H), 4.23 (t, J=7.1 Hz, 2H), 3.33-3.24 (m, 2H), 2.46 (s, 3H), 1.96 (s, 3H), 1.70-1.58 (m, 2H), 1.20-1.04 (m, 7H), 0.74 (t, J=6.9 Hz, 3H). MS (ESI), m/z for C₃₁H₃₆N₄O₄S ([M+H]⁺): Calcd 560.71, found 561.8.

### Example 77 2-acetylamino-2-(4-(ethylsulfonyl)phenyl)-N-(4-(6-methyl-1-((R)-1-(p-tolyl) ethyl)-1H-benzo[d]imidazopyridin-2-yl)phenyl)acetamide

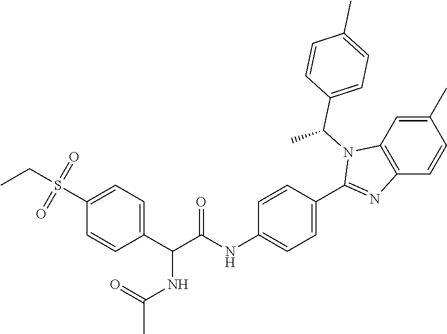 [SMILES: CCS(=O)(=O)c1ccc(C(NC(C)=O)C(=O)Nc2ccc(-c3nc4ccc(C)cc4n3[C@H](C)c3ccc(C)cc3)cc2)cc1]

A synthesis method was as that in Example 73, and a white solid was obtained with a yield of 45%. ¹H NMR (400 MHz, DMSO-d₆) δ 10.75 (s, 1H), 8.90 (d, J=7.8 Hz, 1H), 7.91 (d, J=8.4 Hz, 2H), 7.80-7.73 (m, 4H), 7.61 (d, J=8.6 Hz, 2H), 7.53 (d, J=8.2 Hz, 1H), 7.13 (d, J=8.1 Hz, 2H), 7.04 (d, J=7.9 Hz, 2H), 6.98 (d, J=8.6 Hz, 1H), 6.94 (s, 1H), 5.82 (d, J=7.8 Hz, 1H), 5.74 (q, J=6.9 Hz, 1H), 3.28 (q, J=7.4 Hz, 2H), 2.29 (s, 3H), 2.25 (s, 3H), 1.95 (s, 3H), 1.91 (d, J=7.1 Hz, 3H), 1.09 (t, J=7.3 Hz, 3H). MS (ESI), m/z for C₃₅H₃₆N₄O₄S ([M+H]⁺): Calcd 608.76, found 609.7.

### Example 78 2-(4-(2-acetylamino-2-(4-(ethylsulfonyl)phenyl)acetylamino)phenyl)-N-isopropyl-1-((R)-1-(p-tolyl)ethyl)-1H-benzo[d]imidazol-5-formamide

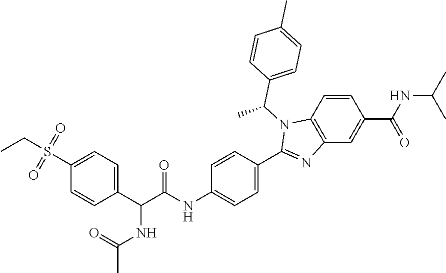 [SMILES: CCS(=O)(=O)c1ccc(C(NC(C)=O)C(=O)Nc2ccc(-c3nc4cc(C(=O)NC(C)C)ccc4n3[C@H](C)c3ccc(C)cc3)cc2)cc1]

A synthesis method was as that in Example 73, and a white solid was obtained with a yield of 46%. ¹H NMR (400 MHz, DMSO-d₆) δ 10.78 (s, 1H), 8.91 (d, J=7.7 Hz, 1H), 8.18 (s, 1H), 8.13 (d, J=8.1 Hz, 1H), 7.92 (d, J=8.4 Hz, 2H), 7.80 (d, J=8.6 Hz, 2H), 7.77 (d, J=8.3 Hz, 2H), 7.68 (d, J=8.5 Hz, 2H), 7.57 (d, J=8.8 Hz, 1H), 7.18-7.09 (m, 3H), 7.04 (d, J=7.8 Hz, 2H), 5.85-5.75 (m, 2H), 4.15-4.03 (m, 1H), 3.33-3.24 (m, 2H), 2.24 (s, 3H), 1.98-1.91 (m, 6H), 1.15 (d, J=6.6 Hz, 6H), 1.09 (t, J=7.3 Hz, 3H). MS (ESI), m/z for C₃₈H₄₁N₅O₅S ([M+H]⁺): Calcd 679.84, found 680.8.

### Example 79 2-(4-(2-acetylamino-2-(4-(ethylsulfonyl)phenyl)acetylamino)phenyl)-N-isopropyl-1-((S)-1-(p-tolyl)ethyl)-1H-benzo[d]imidazol-5-formamide

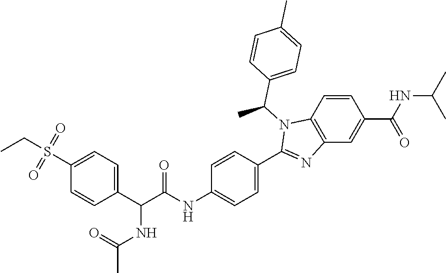 [SMILES: CCS(=O)(=O)c1ccc(C(NC(C)=O)C(=O)Nc2ccc(-c3nc4cc(C(=O)NC(C)C)ccc4n3[C@@H](C)c3ccc(C)cc3)cc2)cc1]

A synthesis method was as that in Example 73, and a white solid was obtained with a yield of 40%. ¹H NMR (400 MHz, DMSO-d₆) δ 10.79 (s, 1H), 8.91 (d, J=7.8 Hz, 1H), 8.18 (s, 1H), 8.13 (d, J=7.7 Hz, 1H), 7.92 (d, J=8.4 Hz, 2H), 7.80 (d, J=8.6 Hz, 2H), 7.77 (d, J=8.4 Hz, 2H), 7.68 (d, J=8.6 Hz, 2H), 7.57 (dd, J=8.6, 1.3 Hz, 1H), 7.17-7.09 (m, 3H), 7.04 (d, J=7.7 Hz, 2H), 5.86-5.75 (m, 2H), 4.15-4.03 (m, 1H), 3.33-3.23 (m, 2H), 2.24 (s, 3H), 1.99-1.91 (m, 6H), 1.16 (d, J=6.6 Hz, 6H), 1.09 (t, J=7.3 Hz, 3H). MS (ESI), m/z for C₃₈H₄₁N₅O₅S ([M+Na]+): Calcd 679.84, found 702.4.

### Example 80 2-acetylamino-2-(4-(ethylsulfonyl)phenyl)-N-(6-(6-methyl-1-((S)-1-(p-tolyl) ethyl)-1H-benzo[d]imidazol-2-yl)pyridin-3-yl)acetamide

### Step 1: synthesis of (S)-5-methyl-N¹-(1-(p-tolyl)ethyl)benzene-1,2-diamine

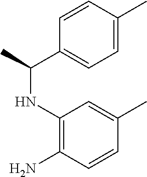 [SMILES: Cc1ccc([C@H](C)Nc2cc(C)ccc2N)cc1]

3-Fluoro-4-nitrotoluene and (S)-1-(4-methylphenyl)ethylamine were used as raw materials. For a synthesis method, reference was made to steps 1 and 2 in Example 31. A yellow-purple liquid was obtained with a yield of 90%. ¹H NMR (400 MHz, DMSO-d₆) δ 7.24 (d, J=7.5 Hz, 2H), 7.08 (d, J=7.4 Hz, 2H), 6.40 (d, J=7.6 Hz, 1H), 6.13 (d, J=7.4 Hz, 1H), 6.02 (s, 1H), 4.71 (d, J=6.2 Hz, 1H), 4.52-4.29 (m, 3H), 2.24 (s, 3H), 1.94 (s, 3H), 1.41 (d, J=6.5 Hz, 3H).

### Step 2: synthesis of (S)—N-(4-methyl-2-((1-(p-tolyl)ethyl)amino)phenyl)-5-nitropyridine

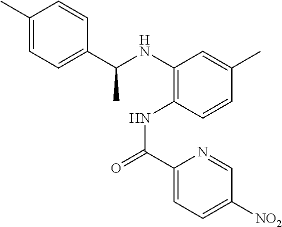 [SMILES: Cc1ccc([C@H](C)Nc2cc(C)ccc2NC(=O)c2ccc([N+](=O)[O-])cn2)cc1]

(S)-5-methyl-N¹-(1-(p-tolyl)ethyl)benzene-1,2-diamine and 5-nitro-2-pyridinecarboxylic acid were used as raw materials. For a synthesis method, reference was made to step 5 in Example 31. A yellow solid was obtained with a yield of 75%. ¹H NMR (400 MHz, DMSO-d₆) δ 10.34 (s, 1H), 9.47 (d, J=2.4 Hz, 1H), 8.81 (dd, J=8.6, 2.5 Hz, 1H), 8.38 (d, J=8.6 Hz, 1H), 7.31 (d, J=7.9 Hz, 2H), 7.21 (d, J=7.9 Hz, 1H), 7.09 (d, J=7.8 Hz, 2H), 6.43 (d, J=8.1 Hz, 1H), 6.32 (s, 1H), 5.30 (d, J=6.4 Hz, 1H), 4.52-4.42 (m, 1H), 2.25 (s, 3H), 2.09 (s, 3H), 1.38 (d, J=6.6 Hz, 3H).

### Step 3: synthesis of (S)-6-methyl-2-(5-nitropyridin-2-yl)-1-(1-(p-tolyl)ethyl)-1H-benzo[d]imidazole

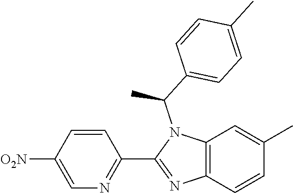 [SMILES: Cc1ccc([C@H](C)n2c(-c3ccc([N+](=O)[O-])cn3)nc3ccc(C)cc32)cc1]

(S)—N-(4-methyl-2-((1-(p-tolyl)ethyl)amino)phenyl)-5-nitropyridine (3.25 g, 8.32 mmol) was dissolved in glacial acetic acid (150 mL), and the solution was refluxed and stirred for 4.5 h at 120° C. After the reaction was finished, the solution was washed with saturated NaHCO₃ until acetic acid was completely eliminated and then extracted multiple times with ethyl acetate. The organic phases were combined, washed once with saturated brine, dried over anhydrous sodium sulfate, concentrated under reduced pressure and isolated through silica gel column chromatography (PE:EA=5:1, v/v) to obtain a target compound as a bright yellow solid (2.69 g with a yield of 87%). ¹H NMR (400 MHz, DMSO-d₆) δ 9.46 (d, J=2.2 Hz, 1H), 8.76 (dd, J=8.8, 2.6 Hz, 1H), 8.59 (d, J=8.8 Hz, 1H), 7.65 (d, J=8.3 Hz, 1H), 7.32 (q, J=6.4 Hz, 1H), 7.21 (d, J=7.9 Hz, 2H), 7.14 (d, J=7.9 Hz, 2H), 7.07 (d, J=8.3 Hz, 1H), 6.99 (s, 1H), 2.30 (s, 3H), 2.26 (s, 3H), 1.98 (d, J=7.1 Hz, 3H).

### Step 4: synthesis of (S)-6-(6-methyl-1-(1-(p-tolyl)ethyl)-1H-benzo[d]imidazol-2-yl)pyridin-3-amine

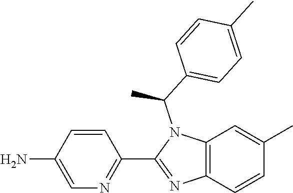 [SMILES: Cc1ccc([C@H](C)n2c(-c3ccc(N)cn3)nc3ccc(C)cc32)cc1]

(S)-6-methyl-2-(5-nitropyridin-2-yl)-1-(1-(p-tolyl)ethyl)-1H-benzo[d]imidazole was used as a raw material. For a synthesis method, reference was made to step 2 in Example 31. A white solid was obtained with a yield of 71%. ¹H NMR (400 MHz, DMSO) δ 8.01-7.95 (m, 2H), 7.47 (d, J=8.2 Hz, 1H), 7.31 (q, J=7.0 Hz, 1H), 7.18 (d, J=8.1 Hz, 2H), 7.12 (d, J=8.1 Hz, 2H), 7.08 (dd, J=8.6, 2.8 Hz, 1H), 6.93 (d, J=8.2 Hz, 1H), 6.85 (s, 1H), 5.80 (s, 2H), 2.25 (s, 6H), 1.90 (d, J=7.1 Hz, 3H).

### Step 5: synthesis of tert-butyl(1-(4-(ethylsulfonyl)phenyl)-2-((6-(6-methyl-1-((S)-1-(p-tolyl) ethyl)-1H-benzo[d]imidazol-2-yl)pyridin-3-yl)amino)-2-oxoethyl)carbamate

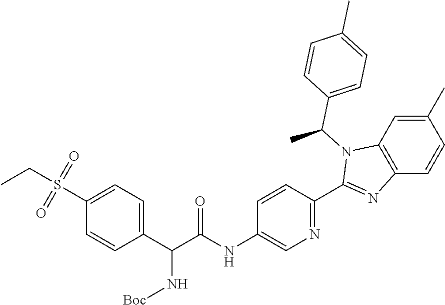 [SMILES: CCS(=O)(=O)c1ccc(C(NC(=O)OC(C)(C)C)C(=O)Nc2ccc(-c3nc4ccc(C)cc4n3[C@@H](C)c3ccc(C)cc3)nc2)cc1]

(S)-5-(6-methyl-1-(1-(p-tolyl)ethyl)-1H-benzo[d]imidazol-2-yl)pyridin-2-amine and 2-((tert-butyloxycarbonyl)amino)-2-(4-(ethylsulfonyl)phenyl)acetic acid were used as raw materials. For a synthesis method, reference was made to step 5 in Example 31. A white solid was obtained with a yield of 39%. ¹H NMR (400 MHz, DMSO-d₆) δ 10.84 (s, 1H), 8.86 (d, J=1.5 Hz, 1H), 8.29-8.20 (m, 2H), 7.94-7.85 (m, 3H), 7.78 (d, J=8.3 Hz, 2H), 7.55 (d, J=8.3 Hz, 1H), 7.26-7.16 (m, 3H), 7.12 (d, J=7.8 Hz, 2H), 6.99 (d, J=8.3 Hz, 1H), 6.92 (s, 1H), 3.33-3.22 (m, 2H), 2.27 (s, 3H), 2.25 (s, 3H), 1.92 (dd, J=6.9, 2.7 Hz, 3H), 1.40 (s, 9H), 1.09 (t, J=7.3 Hz, 3H).

### Step 6: synthesis of 2-amino-2-(4-(ethylsulfonyl)phenyl)-N-(6-(6-methyl-1-((S)-1-(p-tolyl) ethyl)-1H-benzo[d]imidazol-2-yl)pyridin-3-yl)acetamide

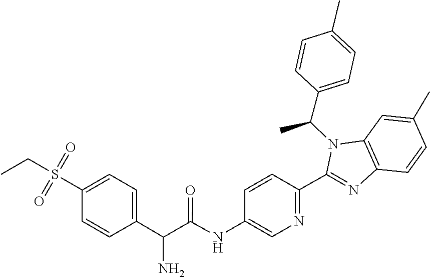 [SMILES: CCS(=O)(=O)c1ccc(C(N)C(=O)Nc2ccc(-c3nc4ccc(C)cc4n3[C@@H](C)c3ccc(C)cc3)nc2)cc1]

Tert-butyl(1-(4-(ethylsulfonyl)phenyl)-2-((6-(6-methyl-1-((S)-1-(p-tolyl)ethyl)-1H-benzo[d]imidazol-2-yl)pyridin-3-yl)amino)-2-oxoethyl)carbamate was used as a raw material. For a synthesis method, reference was made to step 2 in Example 72. A white solid was obtained with a yield of 80%. MS (ESI), m/z for C₃₂H₃₃N₅O₃S ([M+H]⁺): Calcd 567.71, found 568.8.

### Step 7: synthesis of 2-acetylamino-2-(4-(ethylsulfonyl)phenyl)-N-(6-(6-methyl-1-((S)-1-(p-tolyl) ethyl)-1H-benzo[d]imidazol-2-yl)pyridin-3-yl)acetamide

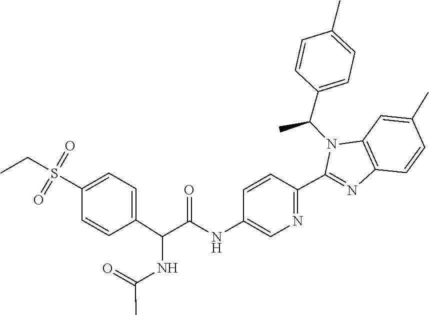 [SMILES: CCS(=O)(=O)c1ccc(C(NC(C)=O)C(=O)Nc2ccc(-c3nc4ccc(C)cc4n3[C@@H](C)c3ccc(C)cc3)nc2)cc1]

2-Amino-2-(4-(ethylsulfonyl)phenyl)-N-(6-(6-methyl-1-((S)-1-(p-tolyl)ethyl)-1H-benzo[d]imidazol-2-yl)pyridin-3-yl)acetamide was used as a raw material. For a synthesis method, reference was made to step 3 in Example 72. A white solid was obtained with a yield of 70%. ¹H NMR (400 MHz, DMSO-d₆) δ 10.96 (s, 1H), 8.95 (d, J=7.7 Hz, 1H), 8.87 (s, 1H), 8.31-8.19 (m, 2H), 7.92 (d, J=8.3 Hz, 2H), 7.77 (d, J=8.3 Hz, 2H), 7.55 (d, J=8.2 Hz, 1H), 7.26-7.15 (m, 3H), 7.12 (d, J=7.8 Hz, 2H), 6.99 (d, J=8.0 Hz, 1H), 6.92 (s, 1H), 5.83 (d, J=7.6 Hz, 1H), 3.33-3.23 (m, 2H), 2.27 (s, 3H), 2.25 (s, 3H), 1.96 (s, 3H), 1.92 (dd, J=7.0, 1.7 Hz, 3H), 1.09 (t, J=7.3 Hz, 3H). MS (ESI), m/z for C₃₄H₃₅N₅O₄S ([M+H]⁺): Calcd 609.75, found 610.7.

### Example 81 2-acetylamino-2-(4-(ethylsulfonyl)phenyl)-N-(6-(6-methyl-1-pentyl-1H-benzo[d]imidazol-2-yl)pyridin-3-yl)acetamide

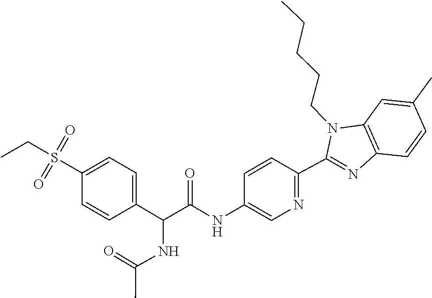 [SMILES: CCCCCn1c(-c2ccc(NC(=O)C(NC(C)=O)c3ccc(S(=O)(=O)CC)cc3)cn2)nc2ccc(C)cc21]

A synthesis method was as that in Example 80, and a white solid was obtained with a yield of 63%. ¹H NMR (400 MHz, DMSO-d₆) δ 10.91 (s, 1H), 8.97-8.88 (m, 2H), 8.27 (d, J=8.5 Hz, 1H), 8.18 (d, J=7.7 Hz, 1H), 7.93 (d, J=7.8 Hz, 2H), 7.78 (d, J=7.9 Hz, 2H), 7.55 (d, J=8.0 Hz, 1H), 7.42 (s, 1H), 7.07 (d, J=7.8 Hz, 1H), 5.82 (d, J=7.3 Hz, 1H), 4.81-4.67 (m, 2H), 3.33-3.23 (m, 2H), 2.47 (s, 3H), 1.97 (s, 3H), 1.79-1.67 (m, 2H), 1.31-1.20 (m, 4H), 1.10 (t, J=7.2 Hz, 3H), 0.81 (t, J=6.4 Hz, 3H). MS (ESI), m/z for C₃₀H₃₅N₅O₄S ([M+H]⁺): Calcd 561.70, found 562.7.

### Example 82 2-acetylamino-2-(4-(ethylsulfonyl)phenyl)-N-(6-(6-methyl-1-((R)-1-(p-tolyl) ethyl)-1H-benzo[d]imidazol-2-yl)pyridin-3-yl)acetamide

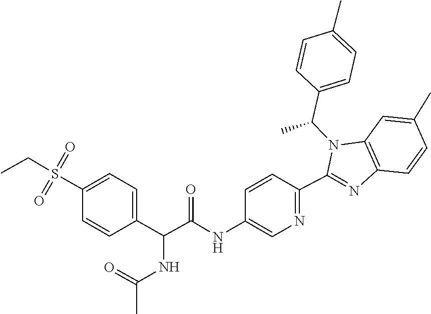 [SMILES: CCS(=O)(=O)c1ccc(C(NC(C)=O)C(=O)Nc2ccc(-c3nc4ccc(C)cc4n3[C@H](C)c3ccc(C)cc3)nc2)cc1]

A synthesis method was as that in Example 80, and a white solid was obtained with a yield of 65%. ¹H NMR (400 MHz, DMSO-d₆) δ 10.96 (s, 1H), 8.95 (d, J=7.7 Hz, 1H), 8.87 (d, J=1.8 Hz, 1H), 8.30-8.21 (m, 2H), 7.92 (d, J=8.4 Hz, 2H), 7.77 (d, J=8.4 Hz, 2H), 7.55 (d, J=8.2 Hz, 1H), 7.26-7.16 (m, 3H), 7.12 (d, J=8.0 Hz, 2H), 6.99 (d, J=8.3 Hz, 1H), 6.92 (s, 1H), 5.83 (d, J=7.7 Hz, 1H), 3.28 (q, J=7.3 Hz, 2H), 2.27 (s, 3H), 2.25 (s, 3H), 1.96 (s, 3H), 1.92 (dd, J=7.1, 1.9 Hz, 3H), 1.09 (t, J=7.3 Hz, 3H). MS (ESI), m/z for C₃₄H₃₅N₅O₄S ([M+H]⁺): Calcd 609.75, found 610.7.

### Example 83 2-(5-(2-acetylamino-2-(4-(ethylsulfonyl)phenyl)acetylamino)pyridin-2-yl)-N-isopropyl-1-((R)-1-(p-tolyl)ethyl)-1H-benzo[d]imidazol-5-formamide

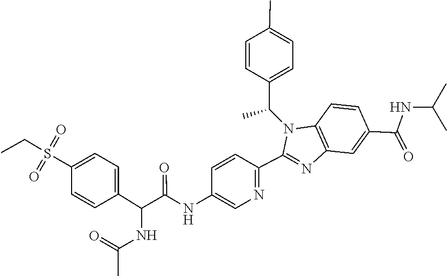 [SMILES: CCS(=O)(=O)c1ccc(C(NC(C)=O)C(=O)Nc2ccc(-c3nc4cc(C(=O)NC(C)C)ccc4n3[C@H](C)c3ccc(C)cc3)nc2)cc1]

A synthesis method was as that in Example 80, and a white solid was obtained with a yield of 49%. ¹H NMR (400 MHz, DMSO-d₆) δ 11.00 (s, 1H), 8.95 (d, J=7.7 Hz, 1H), 8.92 (d, J=2.1 Hz, 1H), 8.34-8.23 (m, 2H), 8.20 (d, J=0.8 Hz, 1H), 8.12 (d, J=7.7 Hz, 1H), 7.92 (d, J=8.4 Hz, 2H), 7.77 (d, J=8.4 Hz, 2H), 7.55 (dd, J=8.6, 1.4 Hz, 1H), 7.25 (q, J=7.0 Hz, 1H), 7.18 (d, J=8.1 Hz, 2H), 7.15-7.07 (m, 3H), 5.84 (d, J=7.7 Hz, 1H), 4.14-4.03 (m, 1H), 3.33-3.24 (m, 2H), 2.24 (s, 3H), 1.99-1.91 (m, 6H), 1.15 (d, J=6.5 Hz, 6H), 1.09 (t, J=7.3 Hz, 3H). MS (ESI), m/z for C₃₇H₄₀N₆O₅S ([M+Na]+): Calcd 680.82, found 703.3.

### Example 84 2-(5-(2-acetylamino-2-(4-(ethylsulfonyl)phenyl)acetylamino)pyridin-2-yl)-N-isopropyl-1-((S)-1-(p-tolyl)ethyl)-1H-benzo[d]imidazol-5-formamide

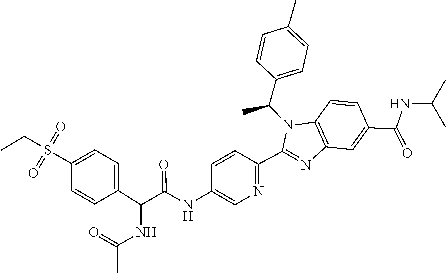 [SMILES: CCS(=O)(=O)c1ccc(C(NC(C)=O)C(=O)Nc2ccc(-c3nc4cc(C(=O)NC(C)C)ccc4n3[C@@H](C)c3ccc(C)cc3)nc2)cc1]

A synthesis method was as that in Example 80, and a white solid was obtained with a yield of 33%. ¹H NMR (400 MHz, DMSO-d₆) δ 11.00 (s, 1H), 8.95 (d, J=7.7 Hz, 1H), 8.91 (d, J=2.2 Hz, 1H), 8.34-8.24 (m, 2H), 8.20 (s, 1H), 8.12 (d, J=7.9 Hz, 1H), 7.92 (d, J=8.3 Hz, 2H), 7.77 (d, J=8.4 Hz, 2H), 7.55 (dd, J=8.7, 1.2 Hz, 1H), 7.25 (q, J=6.8 Hz, 1H), 7.18 (d, J=8.0 Hz, 2H), 7.10 (m, 3H), 5.84 (d, J=8.0 Hz, 1H), 4.13-4.02 (m, 1H), 3.32-3.25 (m, 2H), 2.24 (s, 3H), 1.98-1.90 (m, 6H), 1.15 (d, J=6.5 Hz, 6H), 1.09 (t, J=7.4 Hz, 3H). MS (ESI), m/z for C₃₇H₄₀N₆O₅S ([M−H]⁻): Calcd 680.82, found 679.5.

### Example 85 N-(4-(1-benzyl-5,6-dimethyl-1H-benzo[d]imidazol-2-yl)phenyl)-2-(4-(ethylsulfonyl)phenyl)acetamide

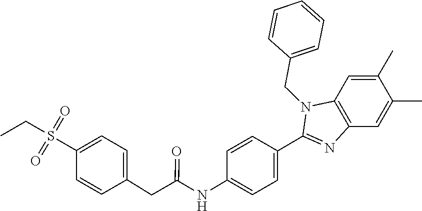 [SMILES: CCS(=O)(=O)c1ccc(CC(=O)Nc2ccc(-c3nc4cc(C)c(C)cc4n3Cc3ccccc3)cc2)cc1]

1-Chloro-4,5-dimethyl-2-nitrobenzene and benzylamine were used as raw materials. For a synthesis method, reference was made to Example 32. A white solid was obtained with a yield of 43%. ¹H NMR (400 MHz, CDCl₃) δ 9.21 (s, 1H), 7.83-7.70 (m, 2H), 7.60-7.50 (m, 3H), 7.49-7.38 (m, 4H), 7.35-7.24 (m, 3H), 7.10-6.96 (m, 3H), 5.36 (s, 2H), 3.77 (s, 2H), 3.14-2.99 (m, 2H), 2.36 (s, 3H), 2.33 (s, 3H), 1.28-1.18 (m, 3H). MS (ESI), m/z for C₃₂H₃₁N₃O₃S ([M+H]⁺): Calcd 537.68, found 538.6.

### Example 86 N-(4-(1-benzyl-5,6-dichloro-1H-benzo[d]imidazol-2-yl)phenyl)-2-(4-(ethylsulfonyl)phenyl)acetamide

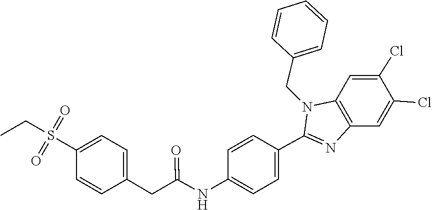 [SMILES: CCS(=O)(=O)c1ccc(CC(=O)Nc2ccc(-c3nc4cc(Cl)c(Cl)cc4n3Cc3ccccc3)cc2)cc1]

1,2-Dichloro-4-fluoro-5-nitrobenzene and benzylamine were used as raw materials. For a synthesis method, reference was made to Example 32. A white solid was obtained with a yield of 47%. ¹H NMR (400 MHz, DMSO-d₆) δ 10.52 (s, 1H), 7.99 (s, 1H), 7.91 (s, 1H), 7.85 (d, J=8.2 Hz, 2H), 7.77-7.68 (m, 4H), 7.61 (d, J=8.2 Hz, 2H), 7.32-7.20 (m, 3H), 6.96 (d, J=7.0 Hz, 2H), 5.63 (s, 2H), 3.84 (s, 2H), 3.27 (q, J=7.3 Hz, 2H), 1.10 (t, J=7.3 Hz, 3H). MS (ESI), m/z for C₃₀H₂₅C₁₂N₃O₃S ([M+H]⁺): Calcd 578.51, found 578.4.

### Example 87 2-(4-(ethylsulfonyl)phenyl)-N-(4-(6-methyl-1H-benzo[d]imidazol-2-yl) phenyl)acetamide

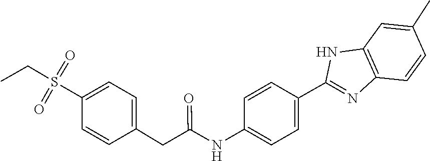 [SMILES: CCS(=O)(=O)c1ccc(CC(=O)Nc2ccc(-c3nc4ccc(C)cc4[nH]3)cc2)cc1]

N-(4-(1-benzyl-6-methyl-1H-benzo[d]imidazol-2-yl)phenyl)-2-(4-(ethylsulfonyl)phenyl)acetamide (100 mg, 0.19 mmol) was dissolved in methanol (20 mL), and Pd—C (100 mg) was added to the mixture. The reaction mixture was stirred overnight at room temperature under hydrogen protection. After the reaction was finished, the reaction mixture was filtered by Celite, concentrated for the solvent to be removed and purified through silica gel column chromatography (PE:EA=4:1, v/v) to obtain a target compound as a white solid (65.9 mg with a yield of 80%). ¹H NMR (400 MHz, DMSO-d₆) δ 10.49 (s, 1H), 8.08 (d, J=8.8 Hz, 2H), 7.86 (d, J=8.3 Hz, 2H), 7.75 (d, J=8.8 Hz, 2H), 7.63 (d, J=8.3 Hz, 2H), 7.44 (d, J=8.2 Hz, 1H), 7.34 (s, 1H), 7.01 (dd, J=8.2, 1.0 Hz, 1H), 3.85 (s, 2H), 3.28 (q, J=7.4 Hz, 2H), 2.42 (s, 3H), 1.10 (t, J=7.4 Hz, 3H). MS (ESI), m/z for C₂₄H₂₃N₃O₃S ([M+H]⁺): Calcd 433.53, found 434.5.

### Test Example 1 In Vitro Intravital Experiment

In the present test example, a protein thermal stability shift assay (TSA) technique was used for detecting abilities of the compounds of the present application to bind to and stabilize RORγ proteins.

Experimental materials: 2 μL of interest protein hRORγ (final concentration: 10 μM), 2 μL of an SYPRO Orange fluorescent stain, 10 μL of each compound (final concentration: 200 μM), 2 μL of a buffer, 4 μL of deionized water, an HSP-96-well reaction plate, and a positive inhibitor: SR2211.

Experimental method: each component was added to the HSP-96-well reaction plate in the above volume, centrifuged at 1000 r/min at room temperature for 1 min and incubated on ice for 30 min. The incubated 96-well reaction plate was placed in a Real-time polymerase chain reaction (PCR) apparatus with a starting temperature of 30° C. and an ending temperature of 80° C. The apparatus was read every five seconds, and the temperature was increased by 0.3° C. per reading. A file was saved, and data was analyzed by GraphPad Prism 7 software.

The luciferase detection technique and TSA detection technique were used for verifying the results, respectively. The results of the luciferase detection technique are shown in Table 1, and the results of the TSA detection technique are shown in Table 2.

TABLE 1

| | |
|---|---|
| A | Examples 26, 32, 34, 35, 36, 37, 38, 42, 43, 44, 45, 46, 47, 48, 49, |
| | 51, 52, 53, 54, 55, 56, 57, 58, 59, 62, 63, 64, 67, 68, 69, 70, 71, 72, |
| | 73, 74, 76, 77, 80, 81 and 82 |
| B | Examples 25, 29, 33, 39, 40, 41, 60, 65, 66, 75, 85 and 86 |
| C | Examples 1, 3, 4, 5, 9, 10, 12, 15, 16, 17, 20, 30, 50, 78, 79, 83, 84 |
| | and 87 |
| D | Examples 2, 6, 7, 8, 11, 13, 14, 18, 19, 21, 22, 23, 24, 27, 28, 31 |
| | and 61 |

Note:
“A” means that IC₅₀ < 0.1 μM,
“B” means that 0.1 μM ≤ IC₅₀ < 1 μM,
“C” means that 1 μM ≤ IC₅₀ < 10 μM, and
“D” means that 10 μM ≤ IC₅₀ <100 μM.

TABLE 2

| | |
|---|---|
| A | Examples 5, 35, 37, 42, 43, 44, 45, 46, 47, 51, 52, 54, 55, 56, 57, |
| | 62, 63, 64, 65, 66, 67, 68, 73, 74, 76, 77, 80, 81, 82, 83, 85 and 86 |
| B | Examples 25, 29, 30, 31, 32, 33, 34, 36, 38, 39, 40, 41, 48, 49, 50, |
| | 53, 58, 59, 60, 69, 70, 71, 72, 78, 79 and 84 |
| C | Examples 1, 2, 3, 4, 6, 7, 8, 9, 10, 11, 12, 13, 14, 15, 16, 17, 18, |
| | 19, 20, 21, 22, 23, 24, 26, 27, 28, 61, 75 and 87 |

Note:
“A” means that ΔTₘ ≤ 5° C.,
“B” means that 5° C. < ΔTₘ < 10° C., and
“C” means that ΔTₘ ≥ 10° C.

The activity data in Tables 1 and 2 indicate that the compounds provided by the present application can better inhibit the transcriptional activity of RORγ and significantly enhance the thermal stability of RORγ protein.

### Test Example 2 Selectivity Test for RORγ and Homologous Proteins Thereof

In the present test example, the selectivity of the compounds in Examples 35, 54, 57, 67, 68 and 74 for RORγ and homologous proteins thereof in the luciferase experiment was evaluated.

Experimental materials: human renal epithelial cell line 293T cells, a DMEM medium containing 10% fetal bovine serum, a 96-well clear plate, a dual reporter gene detection assay kit, an Opti-MEM Reagent, a Lipo-fectamine 2000 Transfection Reagent, recombinant plasmids: Gal4-RORγLBD (25 ng), RORE_Luc (25 ng), pG5-luc and a luciferase (Renilla), and a positive inhibitor: SR2211.

Experimental method: the human renal epithelial cell line 293T cells were cultured in the DMEM containing 10% fetal bovine serum. On the day before transfection, cells were prepared in the 96-well plate with a cell density of 1.5×10⁴ cells/well. After 24 hours of adherent growth, transient transfection was performed with the transfection reagent Lipo-fectamine 2000 through a method of dual reporter gene co-transfection. The transfection reagent and the plasmids were separately diluted with the Opti-MEM Reagent. Gal4-RORγLBD (25 ng), pG5-luc genes (25 ng) and Renilla (5 ng) were added to each well, and after 24 hours of co-transfection, compounds having different concentrations were added. After 24 hours of incubation, the luciferase dual reporter gene detection assay kit was used for detecting luminescence signals. Three duplicate wells were set for each sample, and IC₅₀ values (half maximal inhibitory concentrations) were calculated by software.

Experimental results: the activity data of RORγ and the homologous proteins thereof in Examples 35, 54, 57, 67, 68 and 74 of the present application in the luciferase experiment are shown in Table 3.

TABLE 3

| | Gal4-LBD IC₅₀ (μM) ^{a} | | | | |
|---|---|---|---|---|---|
| Example | RORγ | RORα | RORβ | LXRα | FXR |
| SR2211 | 0.24 ± 0.09 | >20 | >20 | >20 | >20 |
| Example 35 | 0.004 ± 0.0003 | >20 | >20 | >20 | >20 |
| Example 54 | 0.011 ± 0.002 | >20 | >20 | >20 | >20 |
| Example 57 | 0.009 ± 0.004 | >20 | >20 | >20 | >20 |
| Example 67 | 0.024 ± 0.003 | >20 | >20 | >20 | >20 |
| Example 68 | 0.064 ± 0.014 | >20 | >20 | >20 | >20 |
| Example 74 | 0.032 ± 0010 | >20 | >20 | >20 | >20 |

The experimental results indicate that the compounds provided by the present application have specific selectivity for RORγ and, in particular, the compounds in Examples 35, 54, 57, 67, 68 and 74 exhibit more excellent selectivity for RORγ protein relative to proteins of other nuclear receptors.

### Test Example 3 Test for Inhibitory Effects on Proliferation of Cell Lines of AR-Positive Prostate Cancer

In the present test example, inhibitory effects of the compounds in Examples 35, 54, 67, 68 and 74 on proliferation of AR-positive prostate cancer cell lines were evaluated.

Experimental materials: a fluorescence signal detection instrument EnSpire Alpha 2390 Multimode Plate Reader (manufactured by PerkinElmer), a 384-well clear-bottom microplate, a Cell-Titer GLO Luminescence Reagent, prostate cancer cell lines LNCaP, C4-2B and 22Rv1, a medium and fetal bovine serum required for cell culture, and positive drugs: Enzalutamide and SR2211.

Experimental method: 20 μL of a medium containing 500 to 1000 cells to be detected (the actual number of cells was related to cell cycle and cell volume) was plated into each well of the 384-well clear-bottom microplate. After 12 hours, 10 μL of a culture medium containing the compound (the compound had a concentration of 5-100 nM) was added to each well. After incubation with the compound for 72-96 h, the Cell-Titer GLO Reagent was added to each well, and the plate was shaken for 20 min to lyse the cells. After incubation for 10 min, the cells were centrifuged for 1 min, and luminescence 384 signal values were measured. An inhibition curve was fitted by GraphPad Prism software, and IC₅₀ was calculated.

Experimental results: the antiproliferative activity of the compounds in Examples 35, 54, 67, 68 and 74 against the AR-positive prostate cancer cell lines is shown in Table 4.

TABLE 4

| | Cell Viability IC₅₀ (μM)^{a} | | |
|---|---|---|---|
| Cmpd | LNCaP | C4-2B | 22Rv1 |
| Enzalutamide | 42.37 ± 2.37 | 23.56 ± 0.61 | 36.66 ± 4.21 |
| SR2211 | 6.14 ± 0.04 | 5.38 ± 0.27 | 13.34 ± 1.22 |
| Example 35 | 9.22 ± 0.02 | 5.76 ± 0.05 | 14.36 ± 0.46 |
| Example 54 | 4.82 ± 1.08 | 4.40 ± 0.18 | 11.10 ± 0.72 |
| Example 67 | 8.07 ± 0.66 | 6.20 ± 0.60 | 4.62 ± 0.91 |
| Example 68 | 6.33 ± 0.47 | 6.82 ± 0.72 | 8.27 ± 0.75 |
| Example 74 | 5.20 ± 0.14 | 4.19 ± 0.34 | 10.36 ± 1.01 |

As can be seen from the results in Table 4, the compounds provided by the present application may have IC₅₀ values much lower than the currently marketed drug Enzalutamide and have better inhibitory effects on the proliferation of the prostate cancer cells.

### Test Example 4 Pharmacokinetic Evaluations

In the present test example, pharmacokinetic evaluations on Examples 67 and 68 were carried out.

Experimental materials: the pharmacokinetic analyses were carried out by Shanghai Medicilon Corporation. Sprague-Dawley (SD) rats were provided by Shanghai Super-B&K Laboratory Animal Corp., Ltd.

Experimental method: the compound was dissolved in a solution containing 5% dimethylacetamide (DMA), 10% Solutol and 85% Saline as a stock solution. The stock solution was orally administered to three SD rats at a dose of 25 mg/kg and administered to three SD rats through intravenous injection at a single dose of 5 mg/kg. Blood was collected from jugular veins before the oral administration and 0.25, 0.5, 1, 2, 4, 6, 8 and 24 hours after the oral administration. Blood was collected from jugular veins before the intravenous injection and 0.083, 0.25, 0.5, 1, 2, 4, 6, 8 and 24 hours after the intravenous injection. About 200 μL of the blood samples were collected into heparinized tubes and then immediately centrifuged at 8000 r/min for 6 minutes to obtain blood plasma. The obtained blood plasma was storaged at −80° C. until analysis.

Experimental results: the pharmacokinetic data in Examples 67 and 68 are shown in Table 5.

TABLE 5

| | Example 67 | | Example 68 | |
|---|---|---|---|---|
| Parameter | iv (5 mg/kg) | po (25 mg/kg) | iv (5 mg/kg) | po (25 mg/kg) |
| Cmax | 18428.00 ± | 2512.70 ± | 14820.70 ± | 5730.26 ± |
| (μg/L) | 2047.25 | 564.18 | 656.68 | 937.74 |
| Tₘₐₓ (h) | 0.08 ± 0.00 | 4.00 ± 0.00 | 0.08 ± 0.00 | 4.00 ± 0.00 |
| AUC₍₀₋ₜ₎ | 27847.06 ± | 16536.55 ± | 24734.89 ± | 40078.46 ± |
| (μg/L · h) | 1739.28 | 4280.42 | 1990.06 | 11231.62 |
| AUC_{(0-∞)} | 28434.14 ± | 16778.29 ± | 25096.48 ± | 41917.70 ± |
| (μg/L · h) | 1927.93 | 4464.78 | 2142.86 | 12487.47 |
| T_{1/2} (h) | 4.72 ± 0.59 | 3.65 ± 0.58 | 4.19 ± 0.83 | 4.98 ± 0.80 |
| Cl | 0.18 ± 0.01 | — | 0.20 ± 0.02 | — |
| (L/h/kg) | | | | |
| Vz (L/kg) | 1.20 ± 0.10 | — | 1.20 ± 0.20 | — |
| F (%) | — | 11.88 ± 3.07 | — | 32.41 ± 9.08 |

Note:
“—” represents that the data was not measured.

The experimental results indicate that the compounds provided by the present application have good pharmacokinetic properties.

### Test Example 5 In Vivo Pharmacodynamic Study of 22Rv1 Xenograft Mouse Models

Experimental purpose: a xenograft mouse experiment was used for verifying inhibitory effects of the compounds of the present application on tumors in vivo.

Experimental methods: three-week-old male mice (strain: NOD/MrkBomTac-Prkdc^{scid}) were purchased from Beijing Vital River Laboratory Animal Technology Co., Ltd and used for xenograft tumor establishment. 22Rv1 tumor cells were subcutaneously inoculated on a side of a lower abdomen of each mouse, and each mouse was injected with 3×10⁶ cells. The cells were suspended in 100 μL of phosphate-buffered saline (PBS) and Matrigel (at a ratio of 1:1). When the tumor volumes reached about 100 mm³, the mice were randomly divided into groups (n=6-7 per group) and then administered through oral gavage. The compound in Example 68 was dissolved in an administration vehicle containing 15% polyoxyethylene ether (35) castor oil (Cremophor EL), Calbiochem, 82.5% PBS and 2.5% DMSO and administered five days a week for three continuous weeks. The length (L) and width (W) of the tumor mass were measured by a caliper, and the volume was expressed in mm³ and calculated according to the following formula: V=π/6×(L×W²). TGI was calculated according to the following formula: TGI=[1−(T−T₀)/(C−C₀)]×100, where T denoted a mean tumor volume on a specific day of the experiment and T₀ denoted a mean tumor volume at the beginning of the treatment; similarly, C and C₀ denoted a mean tumor volume on a specific day of the experiment and a mean tumor volume of a blank group at the beginning of the treatment, respectively.

In Test Example 5, mouse xenograft tumor models were used for the test in the experiment. 22Rv1 prostate cancer cell xenograft mouse models were selected, the compound in Example 68 was orally administered at a dose of 10 mg/kg or 40 mg/kg five days a week for three weeks, and variations of tumor volumes in mice were observed. Data represented mean tumor volume standard deviation (n=6-7 per group) in each treatment group.

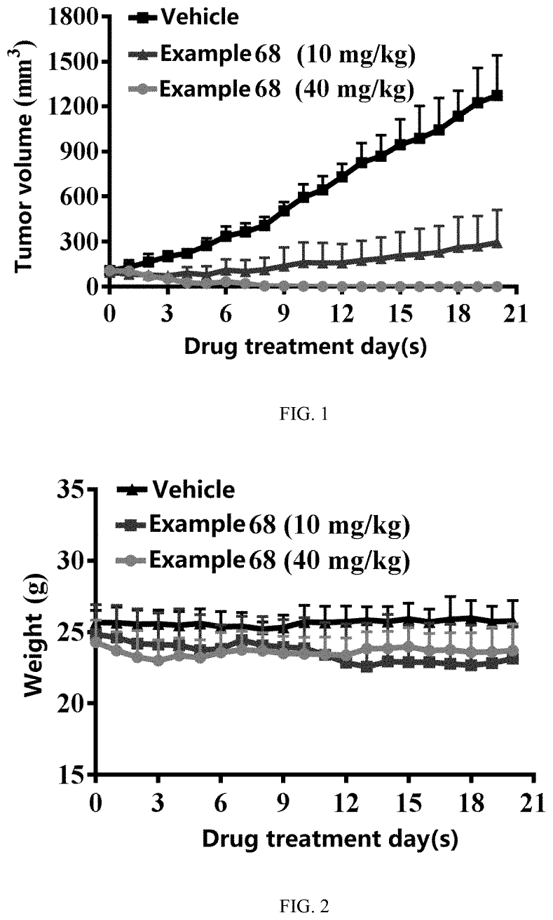

The results of the inhibitory effects of the compound in Example 68 on the tumors in the 22Rv1 mouse xenograft models are shown in FIGS. 1 and 2. FIG. 1 shows that the compound in Example 68 can significantly inhibit the tumors from growing in the mice at a dose of 10 mg/kg (TGI=83%). The compound at a dose of 40 mg/kg can not only significantly inhibit the tumors from growing in the mice but also sustainably and completely eliminate the tumors for a long time (TGI=109%). In addition, FIG. 2 shows that the mice had no apparent variations in weight and behaved normally in the case where the compound in Example 68 was administered at all doses. These results indicate that the compound in Example 68 has a significant inhibitory effect on the tumor growth in vivo on the 22Rv1 mouse xenograft models without any apparent toxic effect.

These compounds effectively bind to and inhibit the transcription of RORγ receptor proteins, thereby inhibiting a downstream signaling pathway and achieving effects such as inhibition of tumor growth and amelioration of inflammation. Example 68 specifically exhibits the inhibitory effect of the compound on the prostate tumor growth in vivo. Available and reported evidences also suggest that this type of compound has a potential to treat diseases such as a cancer, a cell proliferative disorder, an inflammatory disease and an autoimmune disease, sepsis and a viral infection.

The applicant has stated that although the benzo five-membered nitrogen heterocyclic compound and the use thereof in the present application are described through the preceding examples, the present application is not limited to the preceding examples, which means that the implementation of the present application does not necessarily depend on the preceding examples. It is to be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

1. A benzo five-membered nitrogen heterocyclic compound, comprising a structure represented by Formula I: 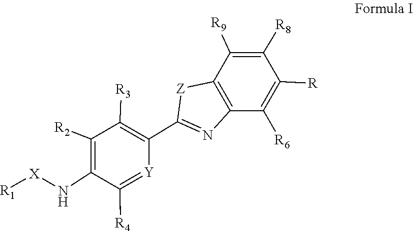
wherein,
in Formula I, X is selected from 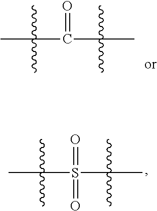
wherein the squiggle represents a bond of the group;
in Formula I, Y is selected from CR₅ or an N atom;
in Formula I, Z is selected from an S atom or NR₁₀;
in Formula I, R₁ and R₁₀ are each independently selected from any one of C1-C10 alkyl substituted with 0 to 3 R₁₁, C6-C10 aryl substituted with 0 to 3 R₁₁, C6-C10 aryl-C1-C3 alkyl substituted with 0 to 3 R₁₁, C2-C10 heteroaryl substituted with 0 to 3 R₁₁, a C2-C20 heterocyclyl substituted with 0 to 3 R₁₁, C2-C10 heteroaryl-C1-C3 alkyl substituted with 0 to 3 R₁₁, a C2-C20 heterocyclyl-C1-C3 alkyl substituted with 0 to 3 R₁₁, C3-C10 cycloalkyl substituted with 0 to 3 R₁₁ or C3-C10 cycloalkyl-C1-C3 alkyl substituted with 0 to 3 R₁₁;
R₁₁ is selected from any one of halogen, cyano, nitro, carboxyl, hydroxyl, amino, C1-C10 alkyl sulfonyl, C1-C10 alkyl sulfonyl substituted with at least one halogen, C1-C10 carbalkoxy, C1-C10 carbalkoxy substituted with at least one halogen, C1-C10 alkyl, C1-C10 alkyl substituted with at least one halogen, C1-C10 alkoxy, C1-C10 alkoxy substituted with at least one halogen, C3-C10 cycloalkyl or C3-C10 cycloalkyl substituted with at least one halogen;
in Formula I, R₂ to R₉ are each independently selected from any one of hydrogen, halogen, C1-C10 alkyl, C1-C10 alkyl substituted with at least one halogen, C1-C10 alkoxy, C1-C10 alkoxy substituted with at least one halogen, C1-C10 alkylamide, C1-C10 alkylamide substituted with at least one halogen, C1-C10 cycloalkylamide, C1-C10 cycloalkylamide substituted with at least one halogen, C1-C10 alkylamino, C1-C10 alkylamino substituted with at least one halogen, C3-C10 cycloalkylamino or C3-C10 cycloalkylamino substituted with at least one halogen.

2. The benzo five-membered nitrogen heterocyclic compound according to claim 1, the C2-C20 heterocyclyl substituted with 0 to 3 R₁₁ is 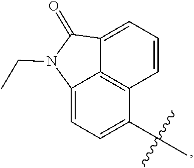 [SMILES: *c1ccc2c3c(cccc13)C(=O)N2CC]
wherein the squiggle represents a bond of the group.

3. The benzo five-membered nitrogen heterocyclic compound according to claim 1, wherein R₁₁ in R₁ is selected from any one or a combination of at least two of C1-C10 alkyl, halogen, trifluoromethoxy, nitro, cyano, carboxyl, methoxycarbonyl, ethoxycarbonyl, amino, methyl sulfonyl or ethyl sulfonyl, preferably, any one or a combination of at least two of cyano, ethoxycarbonyl or ethyl sulfonyl;
optionally, each of R₂ to R₅ is hydrogen;
optionally, R₆, R₇, R₈ and R₉ are not hydrogen at the same time;
optionally, R₆ and R₉ are hydrogen;
optionally, R₇ and R₈ are not hydrogen at the same time;
optionally, one and only one of R₇ and R₈ is hydrogen;
optionally, R₇ and R₈ are each independently selected from any one or a combination of at least two of hydrogen, methyl, methoxy, halogen, trifluoromethyl or C2-C10 alkylamide;
optionally, C2-C10 alkylamide is isopropylamide or cyclopentylamide;
optionally, R₁₀ is selected from any one of C1-C10 alkyl, cyclobutyl, cyclobutylmethyl, cyclohexylmethyl, pyridylmethyl, phenylethyl substituted with 0 to 3 R₁₁ or benzyl substituted with 0 to 3 R₁₁;
optionally, R₁₁ in R₁₀ is selected from any one or a combination of at least two of methyl, carboxyl, trifluoromethyl, methoxycarbonyl, halogen, cyano or methylsulfonyl.

4. The benzo five-membered nitrogen heterocyclic compound according to claim 1, wherein the benzo five-membered nitrogen heterocyclic compound has a structure represented by Formula II: 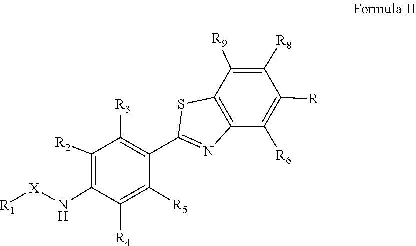
wherein,
in Formula II, X is selected from 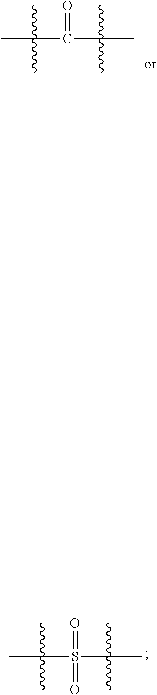
in Formula II, R₁ is selected from any one of C1-C10 alkyl substituted with 0 to 3 R₁₁, C6-C10 aryl substituted with 0 to 3 R₁₁, C6-C10 aryl C1-C3 alkyl substituted with 0 to 3 R₁₁ or a C2-C20 heterocyclyl substituted with 0 to 3 R₁₁; and R₁₁ is selected from any one of halogen, cyano, nitro, carboxyl, hydroxyl, amino, C1-C10 alkyl sulfonyl, C1-C10 alkyl sulfonyl substituted with at least one halogen, C1-C10 carbalkoxy, C1-C10 carbalkoxy substituted with at least one halogen, C1-C10 alkyl, C1-C10 alkyl substituted with at least one halogen, C1-C10 alkoxy or C1-C10 alkoxy substituted with at least one halogen;
in Formula II, R₂ to R₉ are each independently selected from any one of hydrogen, halogen, C1-C10 alkyl, C1-C10 alkyl substituted with at least one halogen, C1-C10 alkoxy or C1-C10 alkoxy substituted with at least one halogen;
optionally, in Formula II, R₂ is selected from hydrogen or chlorine;
optionally, in Formula II, R₈ is selected from any one of methyl, methoxy or fluorine;
optionally, in Formula II, R₇ is hydrogen.

5. The benzo five-membered nitrogen heterocyclic compound according to claim 1, wherein the benzo five-membered nitrogen heterocyclic compound has a structure represented by Formula III: 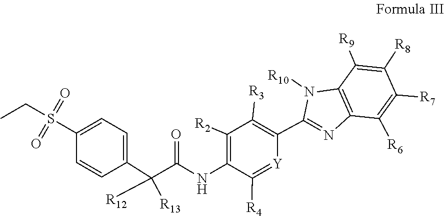
wherein,
in Formula III, Y is selected from CR₅ or an N atom;
in Formula III, R₂ to R₉ are each independently selected from any one of hydrogen, halogen, C1-C10 alkyl, C1-C10 alkyl substituted with at least one halogen, C1-C10 alkoxy, C1-C10 alkoxy substituted with at least one halogen, C1-C10 alkylamide, C1-C10 alkylamide substituted with at least one halogen, C1-C10 cycloalkylamide or C1-C10 cycloalkylamide substituted with at least one halogen;
in Formula III, R₁₀ is selected from any one of C1-C10 alkyl substituted with 0 to 3 R₁₁, C6-C10 aryl-C1-C3 alkyl substituted with 0 to 3 R₁₁, C2-C10 heteroaryl-C1-C3 alkyl substituted with 0 to 3 R₁₁, C3-C10 cycloalkyl substituted with 0 to 3 R₁₁ or C3-C10 cycloalkyl-C1-C3 alkyl substituted with 0 to 3 R₁₁; and R₁₁ is selected from hydrogen, halogen, cyano, carboxyl, C1-C10 alkyl, C1-C10 alkyl substituted with at least one halogen, C1-C10 alkyl sulfonyl, C1-C10 alkyl sulfonyl substituted with at least one halogen, C1-C10 carbalkoxy or C1-C10 carbalkoxy substituted with at least one halogen;
in Formula III, R₁₂ and R₁₃ are each independently selected from hydrogen, amino, C1-C10 alkyl substituted with 0 to 3 R₁₄ or C1-C10 alkylamide substituted with 0 to 3 R₁₄, or R₁₂ and R₁₃ form a C3-C6 carbocyclic ring together with a carbon to which R₁₂ and R₁₃ are joined; and R₁₄ is selected from any one of halogen, carboxyl, hydroxyl, amino, C1-C10 alkylamide, C1-C10 alkylamino or C1-C10 carbalkoxy;
optionally, R₇ is selected from any one of methyl, methoxy, isopropylamide or cyclopentylamide;
optionally, R₈ is selected from any one of methyl, methoxy, trifluoromethyl, isopropylamide or cyclopentylamide;
optionally, R₁₂ and R₁₃ are each independently selected from any one of hydrogen, amino or methylamide.

6. The benzo five-membered nitrogen heterocyclic compound according to claim 1, wherein the benzo five-membered nitrogen heterocyclic compound has any one of the following structures:
4-methyl-N-(4-(6-methylbenzo[d]thiazol-2-yl)phenyl)benzenesulfonamide;
4-(tert-butyl)-N-(4-(6-methylbenzo[d]thiazol-2-yl)phenyl)benzenesulfonamide;
4-fluoro-N-(4-(6-methylbenzo[d]thiazol-2-yl)phenyl)benzenesulfonamide;
N-(4-(6-methylbenzo[d]thiazol-2-yl)phenyl)-4-(trifluoromethoxy)benzenesulfonamide;
N-(4-(6-methylbenzo[d]thiazol-2-yl)phenyl)-4-nitrobenzenesulfonamide;
N-(4-(6-methylbenzo[d]thiazol-2-yl)phenyl)-3-nitrobenzenesulfonamide;
N-(4-(6-methylbenzo[d]thiazol-2-yl)phenyl)-2-nitrobenzenesulfonamide;
N-(4-(6-methylbenzo[d]thiazol-2-yl)phenyl)-3-(methanesulfonyl)benzenesulfonamide;
2,4-difluoro-N-(4-(6-methylbenzo[d]thiazol-2-yl)phenyl)benzenesulfonamide;
2,4,6-trimethyl-N-(4-(6-methylbenzo[d]thiazol-2-yl)phenyl)benzenesulfonamide;
1-ethyl-N-(4-(6-methylbenzo[d]thiazol-2-yl)phenyl)-2-oxo-1,2-dihydrobenzo[cd]indol-6-sulfonamide;
N-(4-(6-methylbenzo[d]thiazol-2-yl)phenyl)-1-(p-tolyl)methanesulfonamide;
1-(4-fluorophenyl)-N-(4-(6-methylbenzo[d]thiazol-2-yl)phenyl)methanesulfonamide;
N-(4-(6-methylbenzo[d]thiazol-2-yl)phenyl)-1-(4-(trifluoromethyl)phenyl)-methanesulfonamide;
N-(4-(6-methylbenzo[d]thiazol-2-yl)phenyl)-1-(4-nitrophenyl)methanesulfonamide;
1-(4-cyanophenyl)-N-(4-(6-methylbenzo[d]thiazol-2-yl)phenyl)methanesulfonamide;
N-(4-(6-methylbenzo[d]thiazol-2-yl)phenyl)-1-(4-(methylsulfonyl)phenyl)-methanesulfonamide;
4-((N-(4-(6-methylbenzo[d]thiazol-2-yl)phenyl)sulfamoyl)methyl)methyl benzoate;
4-((N-(4-(6-methylbenzo[d]thiazol-2-yl)phenyl)sulfamoyl)methyl)benzoic acid;
4-((N-(4-(6-methylbenzo[d]thiazol-2-yl)phenyl)sulfamoyl)methyl)phenyl propionate;
N-(4-(6-methylbenzo[d]thiazol-2-yl)phenyl)-1-(3-nitrophenyl)methanesulfonamide;
N-(4-(6-methylbenzo[d]thiazol-2-yl)phenyl)-1-(2-nitrophenyl)methanesulfonamide;
3-((N-(4-(6-methylbenzo[d]thiazol-2-yl)phenyl)sulfamoyl)methyl)methyl benzoate;
1-(3-aminophenyl)-N-(4-(6-methylbenzo[d]thiazol-2-yl)phenyl)methanesulfonamide;
2-(4-(ethylsulfonyl)phenyl)-N-(4-(6-methylbenzo[d]thiazol-2-yl)phenyl)-acetamide;
N-(4-(6-methylbenzo[d]thiazol-2-yl)phenyl)heptanamide;
N-(4-(6-methylbenzo[d]thiazol-2-yl)phenyl)-2-(p-tolyl)acetamide;
N-(4-(6-methylbenzo[d]thiazol-2-yl)phenyl)-2-(2-nitrophenyl)acetamide;
N-(3-chloro-4-(6-methylbenzo[d]thiazol-2-yl)phenyl)-2-(4-(ethylsulfonyl)-phenyl)acetamide;
2-(4-(ethylsulfonyl)phenyl)-N-(4-(6-fluorobenzo[d]thiazol-2-yl)phenyl)-acetamide;
2-(4-(ethylsulfonyl)phenyl)-N-(4-(6-methoxybenzo[d]thiazol-2-yl)phenyl)-acetamide;
2-(4-(ethylsulfonyl)phenyl)-N-(4-(6-methyl-1-(4-methylphenylethyl)-1H-benzo[d]imidazol-2-yl)phenyl)acetamide;
2-(4-(ethylsulfonyl)phenyl)-N-(4-(6-methyl-1-propyl-1H-benzo[d]imidazol-2-yl)phenyl)acetamide;
N-(4-(1-butyl-6-methyl-1H-benzo[d]imidazol-2-yl)phenyl)-2-(4-(ethylsulfonyl)phenyl)acetamide;
2-(4-(ethylsulfonyl)phenyl)-N-(4-(6-methyl-1-pentyl-1H-benzo[d]imidazol-2-yl)phenyl)acetamide;
2-(4-(ethylsulfonyl)phenyl)-N-(4-(1-isopropyl-6-methyl-1H-benzo[d]imidazol-2-yl)phenyl)acetamide;
2-(4-(ethylsulfonyl)phenyl)-N-(4-(1-isobutyl-6-methyl-1H-benzo[d]imidazol-2-yl)phenyl)acetamide;
2-(4-(ethylsulfonyl)phenyl)-N-(4-(1-isopentyl-6-methyl-1H-benzo[d]imidazole-2-yl)phenyl)acetamide;
N-(4-(1-cyclobutyl-6-methyl-1H-benzo[d]imidazol-2-yl)phenyl)-2-(4-(ethylsulfonyl)phenyl)acetamide;
N-(4-(1-(cyclobutylmethyl)-6-methyl-1H-benzo[d]imidazol-2-yl)phenyl)-2-(4-(ethylsulfonyl)phenyl)acetamide;
N-(4-(1-(cyclohexylmethyl)-6-methyl-1H-benzo[d]imidazol-2-yl)phenyl)-2-(4-(ethylsulfonyl)phenyl)acetamide;
N-(4-(1-benzyl-6-methyl-1H-benzo[d]imidazol-2-yl)phenyl)-2-(4-(ethylsulfonyl)phenyl)acetamide;
2-(4-(ethylsulfonyl)phenyl)-N-(4-(6-methyl-1-(4-methylbenzyl)-1H-benzo[d]imidazol-2-yl)phenyl)acetamide;
2-(4-(ethylsulfonyl)phenyl)-N-(4-(6-methyl-1-phenylethyl-TH-benzo[d]imidazol-2-yl)phenyl)acetamide;
2-(4-(ethylsulfonyl)phenyl)-N-(4-(1-(4-fluorobenzyl)-6-methyl-TH-benzo[d]imidazol-2-yl)phenyl)acetamide;
2-(4-(ethylsulfonyl)phenyl)-N-(4-(1-(3-fluorobenzyl)-6-methyl-TH-benzo[d]imidazol-2-yl)phenyl)acetamide;
2-(4-(ethylsulfonyl)phenyl)-N-(4-(1-(2-fluorobenzyl)-6-methyl-TH-benzo[d]imidazol-2-yl)phenyl)acetamide;
N-(4-(1-(2,6-difluorobenzyl)-6-methyl-TH-benzo[d]imidazol-2-yl)phenyl)-2-(4-(ethylsulfonyl)phenyl)acetamide;
N-(4-(1-(4-cyanobenzyl)-6-methyl-TH-benzo[d]imidazol-2-yl)phenyl)-2-(4-(ethylsulfonyl)phenyl)acetamide;
2-(4-(ethylsulfonyl)phenyl)-N-(4-(6-methyl-1-(4-(methylsulfonyl)benzyl)-1H-benzo[d]imidazol-2-yl)phenyl)acetamide;
2-(4-(ethylsulfonyl)phenyl)-N-(4-(6-methyl-1-(pyridin-2-ylmethyl)-1H-benzo[d]imidazol-2-yl)phenyl)acetamide;
N-(4-(1-benzyl-6-chloro-1H-benzo[d]imidazol-2-yl)phenyl)-2-(4-ethyl-sulfonyl)phenyl)acetamide;
N-(4-(1-benzyl-5-chloro-1H-benzo[d]imidazol-2-yl)phenyl)-2-(4-ethyl-sulfonyl)phenyl)acetamide;
(S)-2-(4-(ethylsulfonyl)phenyl)-N-(4-(6-methyl-1-(1-(p-tolyl)ethyl)-1H-benzo[d]imidazol-2-yl)phenyl)acetamide;
2-(4-(ethylsulfonyl)phenyl)-N-(4-(6-methyl-1-(pyridin-4-ylmethyl)-1H-benzo[d]imidazol-2-yl)phenyl)acetamide;
2-(4-(ethylsulfonyl)phenyl)-N-(4-(6-methyl-1-(1-(p-tolyl)ethyl)-1H-benzo[d]imidazol-2-yl)phenyl)acetamide;
(R)-2-(4-(ethylsulfonyl)phenyl)-N-(4-(6-methyl-1-(1-(p-tolyl)ethyl)-1H-benzo[d]imidazol-2-yl)phenyl)acetamide;
2-(4-(ethylsulfonyl)phenyl)-N-(4-(1-(4-fluorophenethyl)-6-methyl-1H-benzo[d]imidazol-2-yl)phenyl)acetamide;
2-(4-(ethylsulfonyl)phenyl)-N-(4-(6-methyl-1-(4-(trifluoromethyl)benzyl)-1H-benzo[d]imidazol-2-yl)phenyl)acetamide;
4-((2-(4-(2-(4-(ethylsulfonyl)phenyl)acetylamino)phenyl)-6-methyl-1H-benzo[d]imidazol-1-yl)methyl)methyl benzoate;
4-((2-(4-(2-(4-(ethylsulfonyl)phenyl)acetylamino)phenyl)-6-methyl-1H-benzo[d]imidazol-1-yl)methyl)benzoic acid;
2-(4-(ethylsulfonyl)phenyl)-N-(4-(6-methyl-1-(pyridin-3-ylmethyl)-1H-benzo[d]imidazol-2-yl)phenyl)acetamide;
(R)-2-(4-(ethylsulfonyl)phenyl)-N-(4-(1-(1-(p-tolyl)ethyl)-6-(trifluoromethyl)-1H-benzo[d]imidazol-2-yl)phenyl)acetamide;
(R)-2-(4-(ethylsulfonyl)phenyl)-N-(4-(6-methoxy-1-(1-(p-tolyl)ethyl)-1H-benzo[d]imidazol-2-yl)phenyl)acetamide;
(R)-2-(4-(ethylsulfonyl)phenyl)-N-(4-(5-methyl-1-(1-(p-tolyl)ethyl)-1H-benzo[d]imidazol-2-yl)phenyl)acetamide;
(R)-2-(4-(ethylsulfonyl)phenyl)-N-(4-(5-methoxy-1-(1-(p-tolyl)ethyl)-1H-benzo[d]imidazol-2-yl)phenyl)acetamide;
(R)-2-(4-(ethylsulfonyl)phenyl)-N-(4-(1-(1-(4-fluorophenyl)ethyl)-6-(trifluoromethyl)-1H-benzo[d]imidazol-2-yl)phenyl)acetamide;
(S)-2-(4-(ethylsulfonyl)phenyl)-N-(4-(1-(1-(4-fluorophenyl)ethyl)-6-(trifluoromethyl)-1H-benzo[d]imidazol-2-yl)phenyl)acetamide;
(R)-2-(4-(2-(4-(ethylsulfonyl)phenyl)acetylamino)phenyl)-N-isopropyl-1-(1-(p-tolyl)ethyl)-1H-benzo[d]imidazol-6-formamide;
(R)—N-cyclopentyl-2-(4-(2-(4-(ethylsulfonyl)phenyl)acetylamino)phenyl)-1-(1-(p-tolyl)ethyl)-1H-benzo[d]imidazol-6-formamide;
(R)-2-(4-(2-(4-(ethylsulfonyl)phenyl)acetylamino)phenyl)-N-isopropyl-1-(1-(p-tolyl)ethyl)-1H-benzo[d]imidazol-5-formamide;
(R)—N-cyclopentyl-2-(4-(2-(4-(ethylsulfonyl)phenyl)acetylamino)phenyl)-1-(1-(p-tolyl)ethyl)-1H-benzo[d]imidazol-5-formamide;
2-acetylamino-2-(4-(ethylsulfonyl)phenyl)-N-(4-(6-methyl-1-((S)-1-(p-tolyl)ethyl)-1H-benzo[d]imidazol-2-yl)phenyl)acetamide;
2-acetylamino-2-(4-(ethylsulfonyl)phenyl)-N-(4-(6-methyl-1-pentyl-1H-benzo[d]imidazol-2-yl)phenyl)acetamide;
2-acetylamino-2-(4-(ethylsulfonyl)phenyl)-N-(4-(6-methyl-1-((R)-1-(p-tolyl)ethyl)-1H-benzo[d]imidazopyridin-2-yl)phenyl)acetamide;
2-(4-(2-acetylamino-2-(4-(ethylsulfonyl)phenyl)acetylamino)phenyl)-N-isopropyl-1-((R)-1-(p-tolyl)ethyl)-1H-benzo[d]imidazol-5-formamide;
2-(4-(2-acetylamino-2-(4-(ethylsulfonyl)phenyl)acetylamino)phenyl)-N-isopropyl-1-((S)-1-(p-tolyl)ethyl)-1H-benzo[d]imidazol-5-formamide;
2-acetylamino-2-(4-(ethylsulfonyl)phenyl)-N-(6-(6-methyl-1-((S)-1-(p-tolyl)ethyl)-1H-benzo[d]imidazol-2-yl)pyridin-3-yl)acetamide;
2-acetylamino-2-(4-(ethylsulfonyl)phenyl)-N-(6-(6-methyl-1-pentyl-TH-benzo[d]imidazol-2-yl)pyridin-3-yl)acetamide;
2-acetylamino-2-(4-(ethylsulfonyl)phenyl)-N-(6-(6-methyl-1-((R)-1-(p-tolyl)ethyl)-1H-benzo[d]imidazol-2-yl)pyridin-3-yl)acetamide;
2-(5-(2-acetylamino-2-(4-(ethylsulfonyl)phenyl)acetylamino)pyridin-2-yl)-N-isopropyl-1-((R)-1-(p-tolyl)ethyl)-1H-benzo[d]imidazol-5-formamide;
2-(5-(2-acetylamino-2-(4-(ethylsulfonyl)phenyl)acetylamino)pyridin-2-yl)-N-isopropyl-1-((S)-1-(p-tolyl)ethyl)-1H-benzo[d]imidazol-5-formamide;
N-(4-(1-benzyl-5,6-dimethyl-TH-benzo[d]imidazol-2-yl)phenyl)-2-(4-(ethyl-sulfonyl)phenyl)acetamide;
N-(4-(1-benzyl-5,6-dichloro-1H-benzo[d]imidazol-2-yl)phenyl)-2-(4-(ethyl-sulfonyl)phenyl)acetamide; or
2-(4-(ethylsulfonyl)phenyl)-N-(4-(6-methyl-TH-benzo[d]imidazol-2-yl)phenyl)acetamide.

7. A pharmaceutically acceptable salt, isomer, racemate, prodrug, co-crystal complex or solvate of the benzo five-membered nitrogen heterocyclic compound according to claim 1.

8-11. (canceled)

12. A pharmaceutical composition, wherein an active ingredient of the pharmaceutical composition comprises the benzo five-membered nitrogen heterocyclic compound according to claim 1.

13-14. (canceled)

15. A method for preparing an RORγ receptor inhibitor by using the benzo five-membered nitrogen heterocyclic compound according to claim 1.

16. The method according to claim 15, wherein the RORγ receptor inhibitor is used for preparing a drug for treating a cancer, a cell proliferative disorder, an inflammatory disease and an autoimmune disease, sepsis, a viral infection or a neurodegenerative disease.

17. The method according to claim 15, wherein the RORγ receptor inhibitor is used for preparing a drug for treating a cancer.

18. The method according to claim 15, wherein the RORγ receptor inhibitor is used for preparing a drug for treating prostate cancer.

19. A method for treating, preventing or ameliorating an inflammation, an autoimmune disease, a cell proliferative disorder, sepsis, a cancer, a viral infection or a neurodegenerative disease, comprising administering an effective amount of the pharmaceutical composition according to claim 12 to subject in need thereof.

20. The method according to claim 19, wherein the cancer comprises prostate cancer.
